(19) United States
(12) Patent Application Publication
(10) Pub. No.: US 2025/0269019 A1
(43) Pub. Date: Aug. 28, 2025

(54) ANTI-GALECTIN-9 ANTIBODIES AND THERAPEUTIC USES THEREOF

(71) Applicants: PURETECH LYT, INC., Boston, MA (US); BEIGENE SWITZERLAND GMBH, Basel (CH)

(72) Inventors: Aleksandra FILIPOVIC, Boston, MA (US); Eric ELENKO, Boston, MA (US); Heather PADEN, Boston, MA (US); Christopher KORTH, Boston, MA (US); Srikumar SAHASRANAMAN, Basel (CH); Nageshwar BUDHA, Basel (CH)

(73) Assignees: PURETECH LYT, INC., Boston, MA (US); BEIGENE SWITZERLAND GMBH, Basel (CH)

(21) Appl. No.: 18/697,500

(22) PCT Filed: Sep. 30, 2022
(86) PCT No.: PCT/US2022/077408

§ 371 Date: Apr. 1, 2024

Related U.S. Application Data
(60) Provisional application No. 63/277,384, filed on Nov. 9, 2021.
(60) Provisional application No. 63/251,227, filed on Oct. 1, 2021.

(51) Int. Cl. A61K 39/395; A61K 39/00; A61P 35/00; C07K 16/28

(52) U.S. Cl. CPC A61K 39/39558; A61P 35/00; C07K 16/2818; C07K 16/2851; A61K 2039/507; C07K 2317/94

(57) ABSTRACT

Methods for treating solid tumors (e.g., head and neck cancer, urothelial carcinoma, etc.) using an anti-Galectin-9 antibody (e.g., G9.2-17(IgG4)) in combination with tislelizumab.

### CROSS REFERENCE TO RELATED APPLICATIONS

This application is a U.S. National Phase application under 35 U.S.C. § 371 of International Application No. PCT/US2022/077408, filed Sep. 30, 2022, which claims the benefit under 35 U.S.C. § 119(e) of U.S. Provisional Application No. 63/251,227, filed Oct. 1, 2021, and U.S. Provisional Application No. 63/277,384, filed Nov. 9, 2021, the contents of each of which are incorporated by reference herein in their entirety.

### SEQUENCE LISTING

The instant application contains a Sequence Listing which has been filed electronically in XML format and is hereby incorporated by reference in its entirety. Said XML copy, created on Sep. 30, 2022, is named 112174-0234-NP003WO01_SEQ.xml and is 25,891 bytes in size.

### BACKGROUND OF INVENTION

The immune system holds remarkable potential to recognize and destroy cancer cells, but the complex network governing tumor immune escape is an obstacle to broadly effective immune modulation (Martinez-Bosch N, et al., Immune Evasion in Pancreatic Cancer: From Mechanisms to Therapy. Cancers (Basel). 2018; 10 (1)). Approved immuno-oncology (IO) agents deliver incremental survival improvements to many tumor types (e.g., melanoma, lung, renal, bladder cancer, some colon cancers etc.), and are being rapidly integrated as standard of care in addition to and in conjunction with surgery, chemotherapy, and radiotherapy. However, there is still a major gap in the treatment and survivorship of multiple other aggressive malignancies. For example, metastatic pancreatic ductal adenocarcinoma (PDAC or PDA)), cholangiocarcinoma (CCA) and colorectal cancer (CRC) still have 5-year survival rates of <9%, <5% and <15%, respectively. These gastrointestinal tumors are very aggressive, many patients have advanced-stage disease at presentation, and the effectiveness of approved immunotherapies is suboptimal (Rizvi, et al., Cholangiocarcinoma—evolving concepts and therapeutic strategies; Nat Rev Clin Oncol. 2018; 15(2):95-111; Kalyan, et al., Updates on immunotherapy for colorectal cancer; J Gastrointest Oncol. 2018; 9(1):160-169).

The success of first-generation checkpoint inhibitors (anti-PD-1, anti-PD-L1, and anti-CTLA4) has led to an explosion of new IO clinical trial efficacy and differentiation (Holl et al., Examining Peripheral and Tumor Cellular Immunome in Patients with Cancer; Front Immunol. 2019; 10:1767). However, among successes, there have also been many unfortunate development failures, consequently, there is still a need for more novel and efficacious treatments.

Galectin-9 is a tandem-repeat lectin consisting of two carbohydrate recognition domains (CRDs) and was discovered and described for the first time in 1997 in patients suffering from Hodgkin's lymphoma (HL) (Tureci et al., J. Biol. Chem. 1997, 272, 6416-6422). Three isoforms exist and can be located within the cell or extracellularly. Elevated Galectin-9 levels have been in observed a wide range of cancers, including melanoma, Hodgkin's lymphoma, hepatocellular, pancreatic, gastric, colon and clear cell renal cell cancers (Wdowiak et al. Int. J. Mol. Sci. 2018, 19, 210). In renal cancer, patients with high Galectin-9 expression showed more advanced progression of the disease with larger tumor size (Kawashima et al.; BJU Int. 2014; 113:320-332). In melanoma, Galectin-9 was expressed in 57% of tumors and was significantly increased in the plasma of patients with advanced melanoma compared to healthy controls (Enninga et al., Melanoma Res. 2016 October; 26(5): 429-441). A number of studies have shown utility for Galectin-9 as a prognostic marker, and more recently as a potential new drug target (Enninga et al., 2016; Kawashima et al. BJU Int 2014; 113: 320-332; Kageshita et al., Int J Cancer. 2002 Jun. 20; 99(6):809-16, and references therein).

Galectin-9 has been described to play an important role in in a number of cellular processes such as adhesion, cancer cell aggregation, apoptosis, and chemotaxis. Recent studies have shown a role for Galectin-9 in immune modulation in support of the tumor, e.g., through negative regulation of Th1 type responses, Th2 polarization and polarization of macrophages to the M2 phenotype. This work also includes studies that have shown that Galectin-9 participates in direct inactivation of T cells through interactions with the T-cell immunoglobulin and mucin protein 3 (TIM-3) receptor (Dardalhon et al., J Immunol., 2010, 185, 1383-1392; Sanchez-Fueyo et al., Nat Immunol., 2003, 4, 1093-1101).

Galectin-9 has also been found to play a role in polarizing T cell differentiation into tumor suppressive phenotypes), as well as promoting tolerogenic macrophage programming and adaptive immune suppression (Daley et al., Nat Med., 2017, 23, 556-567). In mouse models of pancreatic ductal adenocarcinoma (PDAC), blockade of the checkpoint interaction between Galectin-9 and the receptor Dectin-1 found on innate immune cells in the tumor microenvironment (TME) has been shown to increase anti-tumor immune responses in the TME and to slow tumor progression (Daley et al., Nat Med., 2017, 23, 556-567). Galectin-9 also has been found to bind to CD206, a surface marker of M2 type macrophages, resulting in a reduced secretion of CVL22 (MDC), a macrophage derived chemokine which has been associated with longer survival and lower recurrence risk in lung cancer (Enninga et al, J Pathol. 2018 August; 245(4):468-477).

### SUMMARY OF INVENTION

The present disclosure is based, at least in part, on the development of treatment regimen for solid tumors (e.g., metastatic solid tumors) such as head and neck cancer or urothelial carcinomar, either alone or in combination with a checkpoint inhibitor such as an anti-PD-1 antibody (e.g., tislelizumab).

Accordingly, the present disclosure provides, in some aspects, a method for treating a solid tumor, the method comprising administering to a subject in need thereof (a) an effective amount of an antibody that binds human Galectin-9 (anti-Galectin-9 antibody) and (b) an effective amount of an anti-PD-1 antibody such as tislelizumab. In some embodiments, the anti-Galectin-9 antibody may comprise: (i) a light chain variable region (V_{L}) comprising a light chain complementarity determining region 1 (CDR1) set forth as SEQ ID NO: 1, a light chain complementarity determining region 2 (CDR2) set forth as SEQ ID NO: 2, and a light chain complementarity determining region 3 (CDR3) set forth as SEQ ID NO: 3, and (ii) a heavy chain variable region comprising a heavy chain complementarity determining region 1 (CDR1) set forth as SEQ ID NO: 4, a heavy chain complementarity determining region 2 (CDR2) set forth as SEQ ID NO: 5, and a heavy chain complementarity determining region 3 (CDR3) set forth as SEQ ID NO: 6. In some instances, the anti-Galectin-9 antibody (e.g., G9.2-17(IgG4) as disclosed herein) may be administered to the subject at a dose of about 0.2 mg/kg to about 18 mg/kg. In some examples, the anti-Galectin-9 antibody may be administered to the subject once a week.

In some embodiments, the solid tumor is head and neck cancer, urothelial cancer, gastric esophageal cancer, or non-small cell lung cancer. In some embodiments, the solid tumor is a metastatic tumor (e.g., locally advanced or metastatic solid tumor). In some embodiments, the solid tumor is refractory and/or relapsed. In some embodiments, the subject to be treated by any of the methods disclosed herein is a human patient having the solid tumor.

In some embodiments, the anti-Galectin-9 antibody (e.g., G9.2-17(IgG4) as disclosed herein) may be administered to the subject at a dose of about 4 mg/kg to about 18 mg/kg. for example, the anti-Galectin-9 antibody is administered to the subject at a dose of about 4 mg/kg, about 6.3 mg/kg, about 10 mg/kg, about 12 mg/kg, about 14 mg/kg, about 16 mg/kg, or about 18 mg/kg. In one example, the dose of the anti-Galecti-9 antibody is about 6.3 mg/kg. In another example, the dose of the anti-Galectin-9 antibody is about 10 mg/kg. In yet another example, the dose of the anti-Galectin-9 antibody is about 16 mg/kg.

In some specific examples, the anti-Galectin-9 antibody (e.g., G9.2-17(IgG4) as disclosed herein) may be administered to the subject at a dose of about 6.3 mg/kg once a week. In some specific examples, the anti-Galectin-9 antibody (e.g., G9.2-17(IgG4) as disclosed herein) may be administered to the subject at a dose of about 10 mg/kg once a week. In other specific examples, the anti-Galectin-9 antibody (e.g., G9.2-17(IgG4) as disclosed herein) may be administered to the subject at a dose of about 16 mg/kg once a week. Alternatively or in addition, the anti-Galectin-9 antibody may be administered to the subject by intravenous infusion.

In some embodiments, tislelizumab is administered to the subject at a dose of about 200 mg once every 3 weeks, at a dose of about 300 mg every 4 weeks, or at a dose of about 400 mg every six weeks. In one example, tislelizumab is administered to the subject at a dose of about 300 mg every 4 weeks. Alternatively or in addition, the tislelizumab is administered to the subject by intravenous infusion.

In one example, the method disclosed herein comprise administration of the the anti-Galectin-9 antibody (e.g., G9.2-17(IgG4)) at a dose of about 6.3 mg/kg once a week and administration of tislelizumab at a dose of about 300 mg every 4 weeks. Both antibodies may be administered via intravenous infusion.

In one example, the method disclosed herein comprise administration of the the anti-Galectin-9 antibody (e.g., G9.2-17(IgG4)) at a dose of about 10 mg/kg once a week and administration of tislelizumab at a dose of about 300 mg every 4 weeks. Both antibodies may be administered via intravenous infusion.

In one example, the method disclosed herein comprise administration of the the anti-Galectin-9 antibody (e.g., G9.2-17(IgG4)) at a dose of about 16 mg/kg once a week and administration of tislelizumab at a dose of about 300 mg every 4 weeks. Both antibodies may be administered via intravenous infusion.

In some examples, tislelizumab is administered to the subject on a day when the subject receives the anti-Galectin 9 antibody. Alternatively, the administration of tislelizumab and the administration of the anti-Galectin 9 antibody are on two consecutive days. In some examples, the administration of tislelizumab is performed prior to the administration of the anti-Galectin 9 antibody.

In any of the methods disclosed herein, the anti-Galectin-9 antibody may comprise a V_{L} chain comprising the amino acid sequence of SEQ ID NO: 8, and a V_{H} chain comprising the amino acid sequence of SEQ ID NO: 7. In some instances, the anti-Galectin-9 antibody is an IgG1 or IgG4 molecule. For example, the anti-Galectin-9 antibody is an IgG4 molecule having a modified Fc region of human IgG4. In some examples, the modified Fc region of human IgG4 comprises the amino acid sequence of SEQ ID NO: 14. In one example, the anti-Galectin-9 antibody comprises a heavy chain comprising the amino acid sequence of SEQ ID NO: 19 and a light chain comprising the amino acid sequence of SEQ ID NO: 15.

In any of the methods disclosed herein, the subject has undergone one or more prior anti-cancer therapies. In some examples, the one or more prior anti-cancer therapies comprise chemotherapy, immunotherapy, radiation therapy, a therapy involving a biologic agent, or a combination thereof. In some instances, the subject has progressed disease through the one or more prior anti-cancer therapies or is resistant to the one or more prior therapies.

In some embodiments, the subject is a human patient having an elevated level of Galectin-9 relative to a control value. For example, the human patient has an elevated serum or plasma level of Galectin-9 relative to the control value. In some instances, the human patient has cancer cells expressing Galectin-9, immune cells expressing Galectin-9, or both.

Any of the methods disclosed herein may further comprise monitoring occurrence of adverse effects in the subject. Alternatively or in addition, the method may further comprise reducing the dose of the anti-Galectin-9 antibody, the dose of tislelizumab, or both, when an adverse effect occurs.

Also within the scope of the present disclosure are pharmaceutical compositions for use in treating a solid tumor (e.g., those described herein and including metastatic solid tumors), and uses of any of the anti-Galectin-9 antibodies and the anti-PD-1 antibody such as tislelizumab for manufacturing a medicament for treating the solid tumor, wherein the uses disclosed herein, in some embodiments, involve one or more of the treatment conditions (e.g., dose, dosing regimen, administration route, etc.) as also disclosed herein.

The details of one or more embodiments of the invention are set forth in the description below. Other features or advantages of the present invention are apparent from the following drawing and detailed description of several embodiments, and also from the appended claims.

### BRIEF DESCRIPTION OF DRAWINGS

The following drawings form part of the present specification and are included to further demonstrate certain aspects of the present disclosure, which can be better understood by reference to the drawing in combination with the detailed description of specific embodiments presented herein.

FIG. 1 depict graphs showing results of a study in which mice treated with G9.2-17 mIgG2a alone or in combination with uPD-1 mAb. Mice (n=10/group) with orthotopically implanted KPC tumors were treated with commercial uPD-1 (200p g) mAb or G9.2-17 mIg2a (200 μg), or a combination of G9.2-17 and uPD-1, or matched isotype once weekly for three weeks. Tumors were removed and weighed. Each point represents one mouse; *p<0.05; **p<0.01; ***p<0.001; ****p<0.0001; by unpaired Student's t-test.

FIGS. 2A and 2B depict graphs showing the effect of G9.2-17 in a B16F10 subcutaneous syngeneic model. Tumors were engrafted subcutaneously and treated with G9.2-17 IgG1 mouse mAb, anti-PD-1 antibody or a combination of G9.2-17 IgG1 mouse mAb and anti-PD-1 antibody. FIG. 9A depicts a graph showing the effect on tumor volume. FIG. 9B depicts a graph showing intratumoral CD8 T cell infiltration. Results show that intra-tumoral presence effector T cells were enhanced in the combination arm.

FIGS. 3A and 3B include charts showing cholangiocarcinoma patient-derived tumor cultures ex vivo (organoids) treated with G9.2-17. Patient derived tumor cultures ex vivo (organoids) were treated with G9.2-17 or isotype control for three days. Expression of CD44 (FIG. 3A), and TNFα (FIG. 3B) in CD3+ T cells from PDOTS was assessed.

### DETAILED DESCRIPTION OF INVENTION

Provided herein are methods of using anti-Galectin-9 antibodies, e.g., G9.2-17, for treating solid tumors, for example, head and neck cancer, urothelial carcinoma, and other solid tumors as disclosed herein, in combination with a checkpoint inhibitor such as an anti-PD-1 antibody (e.g., tisleilizumab). In some embodiments, the cancers are metastatic. In some embodiments, the methods disclosed herein provide specific doses and/or dosing schedules. In some instances, the methods disclosed herein target specific patient populations, for example, patients who have undergone prior treatment and show disease progression through the prior treatment, or patients who are resistant (de novo or acquired) to the prior treatment.

Galectin-9, a tandem-repeat lectin, is a beta-galactoside-binding protein, which has been shown to have a role in modulating cell-cell and cell-matrix interactions. It is found to be strongly overexpressed in Hodgkin's disease tissue and in other pathologic states. It has in some instances also been found circulating in the tumor microenvironment (TME).

Galectin-9 is found to interact with Dectin-1, an innate immune receptor which is highly expressed on macrophages in PDAC, as well as on cancer cells (Daley, et al. Nat Med. 2017; 23(5):556-6). Regardless of the source of Galectin-9, disruption of its interaction with Dectin-1 has been shown to lead to the reprogramming of CD4+ and CD8+ cells into indispensable mediators of anti-tumor immunity. Thus, Galectin-9 serves as a valuable therapeutic target for blocking the signaling mediated by Dectin-1. Accordingly, in some embodiments, the anti-Galectin-9 antibodies describe herein disrupt the interaction between Galectin-9 and Dectin-1.

Galectin-9 is also found to interact with TIM-3, a type I cell surface glycoprotein expressed on the surface of leukemic stem cells in all varieties of acute myeloid leukemia (except for M3 (acute promyelocytic leukemia)), but not expressed in normal human hematopoietic stem cells (HSCs). TIM-3 signaling resulting from Galectin-9 ligation has been found to have a pleiotropic effect on immune cells, inducing apoptosis in Th1 cells (Zhu et al., Nat Immunol., 2005, 6:1245-1252) and stimulating the secretion of tumor necrosis factor-α (TNF-α), leading to the maturation of monocytes into dendritic cells, resulting in inflammation by innate immunity (Kuchroo et al., Nat Rev Immunol., 2008, 8:577-580). Further Galectin-9/TIM-3 signaling has been found to co-activate NF-κB and β-catenin signaling, two pathways that promote LSC self-renewal (Kikushige et al., Cell Stem Cell, 2015, 17(3):341-352). An anti-Galectin-9 antibody that interferes with Galectin-9/TIM-3 binding could have a therapeutic effect, especially with respect to leukemia and other hematological malignancies. Accordingly, in some embodiments, the anti-Galectin-9 antibodies described herein disrupt the interaction between Galectin-9 and TIM-3.

Further, Galectin-9 is found to interact with CD206, a mannose receptor highly expressed on M2 polarized macrophages, thereby promoting tumor survival (Enninga et al., J Pathol. 2018 August; 245(4):468-477). Tumor-associated macrophages expressing CD206 are mediators of tumor immunosuppression, angiogenesis, metastasis, and relapse (see, e.g., Scodeller et al., Sci Rep. 2017 Nov. 7; 7(1):14655, and references therein). Specifically, M1 (also termed classically activated macrophages) are trigged by Th1-related cytokines and bacterial products, express high levels of IL-12, and are tumoricidal. By contrast, M2 (so-called alternatively activated macrophages) are activated by Th2-related factors, express high level of anti-inflammatory cytokines, such as IL-10, and facilitate tumor progression (Biswas and Mantovani; Nat Immunol. 2010 October; 11(10):889-96). The pro-tumoral effects of M2 include the promotion of angiogenesis, advancement of invasion and metastasis, and the protection of the tumor cells from chemotherapy-induced apoptosis (Hu et al., Tumour Biol. 2015 December; 36(12): 9119-9126, and references therein). Tumor-associated macrophages are thought be of M2-like phenotype and have a protumor role. Galectin-9 has been shown to mediate myeloid cell differentiation toward an M2 phenotype (Enninga et al., Melanoma Res. 2016 October; 26(5):429-41). It is possible that Galectin-9 binding CD206 may result in reprogramming TAMs towards the M2 phenotype, similar to what has been previously shown for Dectin-1. Without wishing to be bound by theory, blocking the interaction of Galectin-9 with CD206 may provide one mechanism by which an anti-Galectin-9 antibody, e.g., a G9.2-17 antibody, can be therapeutically beneficial. Accordingly, in some embodiments, the anti-Galectin-9 antibodies described herein disrupt the interaction between Galectin-9 and CD206.

Galectin-9 has also been shown to interact with protein disulfide isomerase (PDI) and 4-1BB (Bi S, et al. Proc Natl Acad Sci USA. 2011; 108(26):10650-5; Madireddi et al. J Exp Med. 2014; 211(7):1433-48).

Anti-Galectin-9 antibodies can serve as therapeutic agents for treating diseases associated with Galectin-9 (e.g., those in which a Galectin-9 signaling plays a role). Without being bound by theory, an anti-Galectin-9 antibody may block a signaling pathway mediated by Galectin-9. For example, the antibody may interfere with the interaction between Galectin-9 and its binding partner (e.g., Dectin-1, TIM-3 or CD206), thereby blocking the signaling triggered by the Galectin-9/Ligand interaction. Alternatively, or in addition, an anti-Galectin-9 antibody may also exert its therapeutic effect by inducing blockade and/or cytotoxicity, for example, ADCC, CDC, or ADCP against pathologic cells that express Galectin-9. A pathologic cell refers to a cell that contributes to the initiation and/or development of a disease, either directly or indirectly.

The anti-Galectin-9 antibodies disclosed herein are capable of suppressing the signaling mediated by Galectin-9 (e.g., the signaling pathway mediated by Galectin-9/Dectin-1 or Galectin-9/Tim-3) or eliminating pathologic cells expressing Galectin-9 via, e.g., ADCC. Accordingly, the anti-Galectin-9 antibodies described herein can be used for inhibiting any of the Galectin-9 signaling and/or eliminating Galectin-9 positive pathologic cells, thereby benefiting treatment of diseases associated with Galectin-9.

Anti-Galectin-9 antibodies such as G9.2-17 were found to be effective in inducing apoptosis against cells expressing Galectin-9. Further, the anti-tumor effects of anti-Galectin-9 antibodies such as G9.2-17 were demonstrated in a mouse model, either by itself, or in combination with a checkpoint inhibitor (e.g., an anti-PD-1 antibody). As reported herein, the efficacy of G9.2-17 was tested in mouse models of PDAC and melanoma as well as in patient derived organoid tumor models (PDOTs). The orthotopic PDAC KPC mouse model (LSL-KrasG12D/+; LSL-Trp53R172H/+; Pdx-1-Cre) that was used recapitulates many features of human disease, including unresponsiveness to approved checkpoint inhibitors (Bisht and Feldmann G; Animal models for modeling pancreatic cancer and novel drug discovery; Expert Opin Drug Discov. 2019; 14(2):127-142; Weidenhofer et al., Animal models of pancreatic cancer and their application in clinical research; Gastrointestinal Cancer: Targets and Therapy 2016; 6). The B16F10 melanoma mouse model has been a long-standing standard to test immunotherapies (Curran et al., PD-1 and CTLA-4 combination blockade expands infiltrating T cells and reduces regulatory T and myeloid cells within B16 melanoma tumors; Proc Natl Acad Sci USA. 2010; 107(9):4275-4280).

PDOTs isolated from fresh human tumor samples retain autologous lymphoid and myeloid cell populations, including antigen-experienced tumor infiltrating CD4 and CD8 T lymphocytes, and respond to immune therapies in short-term ex vivo culture (Jenkins et al. Ex Vivo Profiling of PD-1 Blockade Using Organotypic Tumor Spheroids. Cancer Discov. 2018; 8(2):196-215; Aref et al., 3D microfluidic ex vivo culture of organotypic tumor spheroids to model immune checkpoint blockade; Lab Chip. 2018; 18(20):3129-3143). As reported herein, expression of Galectin-9 on cancer cells was observed in patient-derived organoid assays.

In vivo studies were performed with G9.2-17 mouse IgG1 (G9.2-17 mIgG1 contains the exact same binding epitope as G9.2-17 human IgG4 and has the same effector function), which achieves significant reduction of tumor growth already as a single agent in the orthotopic KPC model, where approved checkpoint inhibitors do not work. In the B16F10 model G9.2-17 significantly exceeds the efficacy of anti-PD-1. In both models, modulation of the intra-tumoral immune microenvironment using G9.2-17 mIgG1 through the upregulation of effector T cell activity and inhibition of immunosuppressive signals, as well as the augmentation of intra-tumoral CD8 T cell infiltration was demonstrated.

These results demonstrate that the anti-tumor methods disclosed herein, involving an anti-Galectin-9 antibody, optionally in combination the checkpoint inhibitor, would achieve superior therapeutic efficacy against the target solid tumors.

Accordingly, described herein are therapeutic uses of anti-Galectin-9 antibodies for treating certain cancers as disclosed herein.

### Antibodies Binding to Galectin-9

The present disclosure provides anti-Galectin-9 antibody G9.2-17 and functional variants thereof for use in the treatment methods disclosed herein.

An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody”, e.g., anti-Galectin-9 antibody, encompasses not only intact (e.g., full-length) polyclonal or monoclonal antibodies, but also antigen-binding fragments thereof (such as Fab, Fab′, F(ab′)2, Fv), single chain (scFv), mutants thereof, fusion proteins comprising an antibody portion, humanized antibodies, chimeric antibodies, diabodies, nanobodies, linear antibodies, single chain antibodies, multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody, e.g., anti-Galectin-9 antibody, includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

A typical antibody molecule comprises a heavy chain variable region (V_{H}) and a light chain variable region (V_{L}), which are usually involved in antigen binding. The V_{H} and V_{L} regions can be further subdivided into regions of hypervariability, also known as “complementarity determining regions” (“CDR”), interspersed with regions that are more conserved, which are known as “framework regions” (“FR”). Each V_{H} and V_{L} is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, the EU definition, the “Contact” numbering scheme, the IMGT” numbering scheme, the “AHo” numbering scheme, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; Edelman et al., Proc Natl Acad Sci USA. 1969 May; 63(1):78-85; and Almagro, J. Mol. Recognit. 17:132-143 (2004); MacCallum et al., J. Mol. Biol. 262:732-745 (1996), Lefranc M P et al., Dev Comp Immunol, 2003 January; 27(1):55-77; and Honegger A and Pluckthun A, J Mol Biol, 2001 Jun. 8; 309(3):657-70. See also hgmp.mrc.ac.uk and bioinf.org.uk/abs).

In some embodiments, the anti-Galectin-9 antibody described herein is a full-length antibody, which contains two heavy chains and two light chains, each including a variable domain and a constant domain. Alternatively, the anti-Galectin-9 antibody can be an antigen-binding fragment of a full-length antibody. Examples of binding fragments encompassed within the term “antigen-binding fragment” of a full length antibody include (i) a Fab fragment, a monovalent fragment consisting of the V_{L}, V_{H}, C_{L} and C_{H}1 domains; (ii) a F(ab′)₂ fragment, a bivalent fragment including two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR) that retains functionality. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv). See e.g., Bird et al. (1988) Science 242:423-426; and Huston et al. (1988) Proc. Natl. Acad. Sci. USA 85:5879-5883.

Any of the antibodies described herein, e.g., anti-Galectin-9 antibody, can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

Reference antibody G9.2-17 refers to an antibody capable of binding to human Galectin-9 and comprises a heavy chain variable region of SEQ ID NO: 7 and a light chain variable domain of SEQ ID NO: 8, both of which are provided below. In some embodiments, the anti-Galectin-9 antibody for use in the methods disclosed herein is the G9.2-17 antibody. In some embodiments, the anti-Galectin-9 antibody for use in the methods disclosed herein is an antibody having the same heavy chain complementarity determining regions (CDRs) as reference antibody G9.2-17 and/or the same light chain complementarity determining regions as reference antibody G9.2-17. Two antibodies having the same V_{H} and/or V_{L} CDRs means that their CDRs are identical when determined by the same approach (e.g., the Kabat approach, the Chothia approach, the AbM approach, the Contact approach, or the IMGT approach as known in the art. See, e.g., bioinf.org.uk/abs/).

The heavy and light chain CDRs of reference antibody G9.2-17 is provided in Table 1 below (determined using the Kabat methodology):

TABLE 1
Heavy and Light Chain CDRs of G9.2-17

| | | | |
|---|---|---|---|
| G9.2-17 | V_{L} CDR1 | RASQSVSSAVA | SEQ ID NO: 1 |
| | V_{L} CDR2 | SASSLYS | SEQ ID NO: 2 |
| | V_{L} CDR3 | QQSSTDPIT | SEQ ID NO: 3 |
| | V_{H} CDR1 | FTVSSSSIH | SEQ ID NO: 4 |
| | V_{H} CDR2 | YISSSSGYTYYADSVKG | SEQ ID NO: 5 |
| | V_{H} CDR3 | YWSYPSWWPYRGMDY | SEQ ID NO: 6 |

In some examples, the anti-Galectin-9 antibody for use in the methods disclosed herein may comprise (following the Kabat scheme) a heavy chain complementarity determining region 1 (CDR1) set forth as SEQ ID NO: 4, a heavy chain complementarity determining region 2 (CDR2) set forth as SEQ ID NO: 5, and a heavy chain complementarity determining region 3 (CDR3) set forth as SEQ ID NO: 6 and/or may comprise a light chain complementarity determining region 1 (CDR1) set forth as SEQ ID NO: 1, a light chain complementarity determining region 2 (CDR2) set forth as SEQ ID NO: 2, and a light chain complementarity determining region 3 (CDR3) set forth as SEQ ID NO: 3. The anti-Galectin-9 antibody, including the reference antibody G9.2-17, can be in any format as disclosed herein, for example, a full-length antibody or a Fab. The term “G9.2-17(IgG4)” used herein refers to a G9.2-17 antibody which is an IgG4 molecule (e.g., having a heavy chain comprising SEQ ID NO. 19 and a light chain comprising SEQ ID NO: 15). Likewise, the term “G9.2-17 (Fab)” refers to a G9.2-17 antibody, which is a Fab molecule.

In some embodiments, the anti-Galectin-9 antibody or binding portion thereof comprises heavy and light chain variable regions, wherein the light chain variable region CDR1, CDR2, and CDR3 amino acid sequences have at least 80% (e.g., 80%, 85%, 90%, 91%, 92%, 93%, 94%, 95%, 96%, 97%, 98%, or 99% and any increment therein) sequence identity to the light chain variable region CDR1, CDR2, and CDR3 amino acid sequences set forth in SEQ ID NOs: 1, 2, and 3, respectively. In some embodiments, the anti-Galectin-9 antibody or binding portion thereof comprises heavy and light chain variable regions, wherein the heavy chain variable region CDR1, CDR2, and CDR3 amino acid sequences have at least 80% (e.g., 80%, 85%, 90%, 91%, 92%, 93%, 94%, 95%, 96%, 97%, 98%, or 99% and any increment therein) sequence identity to the heavy chain variable region CDR1, CDR2, and CDR3 amino acid sequences set forth in SEQ ID NO: 4, 5, and 6, respectively.

Additional Galectin-9 antibodies, e.g., which bind to the CRD1 and/or CRD2 region of Galectin-9 are described in co-owned, co-pending U.S. patent application Ser. No. 16/173,970 and in co-owned, co-pending International Patent Applications PCT/US18/58028 and PCT/US2020/024767, the contents of each of which are herein incorporated by reference in their entireties.

In some embodiments, the anti-Galectin-9 antibody disclosed herein comprises light chain CDRs that have at least 80% (e.g., 80%, 85%, 90%, 91%, 92%, 93%, 94%, 95%, 96%, 97%, 98%, or 99% and any increment therein) sequence identity, individually or collectively, as compared with the corresponding V_{L} CDRs of reference antibody G9.2-17. Alternatively or in addition, in some embodiments, the anti-Galectin-9 antibody comprises heavy chain CDRs that have at least 80% (e.g., 80%, 85%, 90%, 91%, 92%, 93%, 94%, 95%, 96%, 97%, 98%, or 99% and any increment therein) sequence identity, individually or collectively, as compared with the corresponding V_{H} CDRs of reference antibody G9.2-17.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of the invention. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In other embodiments, the anti-Galectin-9 antibody described herein comprises a V_{H} that comprises the HC CDR1, HC CDR2, and HC CDR3, which collectively contain up to 8 amino acid residue variations (8, 7, 6, 5, 4, 3, 2, or 1 variation(s), including additions, deletions, and/or substitutions) relative to the HC CDR1, HC CDR2, and HC CDR3 of reference antibody G9.2-17. Alternatively or in addition, in some embodiments, the anti-Galectin-9 antibody described herein comprises a V_{H} that comprises the LC CDR1, LC CDR2, and LC CDR3, which collectively contain up to 8 amino acid residue variations (8, 7, 6, 5, 4, 3, 2, or 1 variation(s) including additions, deletions, and/or substitutions) relative to the LC CDR1, LC CDR2, and LC CDR3 of reference antibody G9.2-17.

In one example, the amino acid residue variations are conservative amino acid residue substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

In some embodiments, the anti-Galectin-9 antibodies disclosed herein, having the heavy chain CDRs disclosed herein, contains framework regions derived from a subclass of germline V_{H} fragment. Such germline V_{H} regions are well known in the art. See, e.g., the IMGT database (www.imgt.org) or at www.vbase2.org/vbstat.php. Examples include the IGHV1 subfamily (e.g., IGHV1-2, IGHV1-3, IGHV1-8, IGHV1-18, IGHV1-24, IGHV1-45, IGHV1-46, IGHV1-58, and IGHV1-69), the IGHV2 subfamily (e.g., IGHV2-5, IGHV2-26, and IGHV2-70), the IGHV3 subfamily (e.g., IGHV3-7, IGHV3-9, IGHV3-11, IGHV3-13, IGHV3-15, IGHV3-20, IGHV3-21, IGHV3-23, IGHV3-30, IGHV3-33, IGHV3-43, IGHV3-48, IGHV3-49, IGHV3-53, IGHV3-64, IGHV3-66, IGHV3-72, and IGHV3-73, IGHV3-74), the IGHV4 subfamily (e.g., IGHV4-4, IGHV4-28, IGHV4-31, IGHV4-34, IGHV4-39, IGHV4-59, IGHV4-61, and IGHV4-B), the IGHV subfamily (e.g., IGHV5-51, or IGHV6-1), and the IGHV7 subfamily (e.g., IGHV7-4-1).

Alternatively or in addition, in some embodiments, the anti-Galectin-9 antibody, having the light chain CDRs disclosed herein, contains framework regions derived from a germline VK fragment. Examples include an IGKV1 framework (e.g., IGKV1-05, IGKV1-12, IGKV1-27, IGKV1-33, or IGKV1-39), an IGKV2 framework (e.g., IGKV2-28), an IGKV3 framework (e.g., IGKV3-11, IGKV3-15, or IGKV3-20), and an IGKV4 framework (e.g., IGKV4-1). In other instances, the anti-Galectin-9 antibody comprises a light chain variable region that contains a framework derived from a germline Vλ fragment. Examples include an IGλ1 framework (e.g., IGλV1-36, IGλV1-40, IGλV1-44, IGλV1-47, IGλV1-51), an IGλ2 framework (e.g., IGλV2-8, IGλV2-11, IGλV2-14, IGλV2-18, IGλV2-23), an IGλ3 framework (e.g., IGλV3-1, IGλV3-9, IGλV3-10, IGλV3-12, IGλV3-16, IGλV3-19, IGλV3-21, IGλV3-25, IGλV3-27), an IGλ4 framework (e.g., IGλV4-3, IGλV4-60, IGλV4-69), an IGλ5 framework (e.g., IGλV5-39, IGλV5-45), an IGλ6 framework (e.g., IGλV6-57), an IGλ7 framework (e.g., IGλV7-43, IGλV7-46), an IGλ8 framework (e.g., IGλV8-61), an IGλ9 framework (e.g., IGλV9-49), or an IGλ10 framework (e.g., IGλV10-54).

In some embodiments, the anti-Galectin-9 antibody for use in the method disclosed herein can be an antibody having the same heavy chain variable region (V_{H}) and/or the same light chain variable region (V_{L}) as reference antibody G9.2-17, the V_{H} and V_{L} region amino acid sequences are provided below:

V_{H}:
(SEQ ID NO: 7)
EVQLVESGGGLVQPGGSLRLSCAASGFTVSSSSIHWVRQAPGKGLEWVA
YISSSSGYTYYADSVKGRFTISADTSKNTAYLQMNSLRAEDTAVYYCAR
YWSYPSWWPYRGMDYWGQGTLVTVSS
V_{L}:
(SEQ ID NO: 8)
DIQMTQSPSSLSASVGDRVTITCRASQSVSSAVAWYQQKPGKAPKLLIY
SASSLYSGVPSRFSGSRSGTDFTLTISSLQPEDFATYYCQQSSTDPITF
GQGIKVEIKR

In some embodiments, the anti-Galectin-9 antibody has at least 80% sequence identity (e.g., 80%, 85%, 90%, 91%, 92%, 93%, 94%, 95%, 96%, 97%, 98%, or 99% identity) to the heavy chain variable region of SEQ ID NO: 7. Alternatively or in addition, the anti-Galectin-9 antibody has at least 80% sequence identity (e.g., 80%, 85%, 90%, 91%, 92%, 93%, 94%, 95%, 96%, 97%, 98%, or 99% identity) to the light chain variable region of SEQ ID NO: 8.

In some instances, the anti-Galectin-9 antibody disclosed herein is a functional variant of reference antibody G9.2-17. A functional variant can be structurally similar as the reference antibody (e.g., comprising the limited number of amino acid residue variations in one or more of the heavy chain and/or light chain CDRs as G9.2-17 as disclosed herein, or the sequence identity relative to the heavy chain and/or light chain CDRs of G9.2-17, or the VH and/or VL of G9.2-17 as disclosed herein) with substantially similar binding affinity (e.g., having a K_{D} value in the same order) to human Galectin-9.

In some embodiments, the anti-Galectin-9 antibody as described herein can bind and inhibit the activity of Galectin-9 by at least 20% (e.g., 31%, 35%, 40%, 45%, 50%, 60%, 70%, 80%, 90%, 95% or greater, including any increment therein). The apparent inhibition constant (Ki^{app} or K_{i,app}), which provides a measure of inhibitor potency, is related to the concentration of inhibitor required to reduce enzyme activity and is not dependent on enzyme concentrations. The inhibitory activity of an anti-Galectin-9 antibody described herein can be determined by routine methods known in the art.

The K_{i,}^{app} value of an antibody may be determined by measuring the inhibitory effect of different concentrations of the antibody on the extent of the reaction (e.g., enzyme activity); fitting the change in pseudo-first order rate constant (v) as a function of inhibitor concentration to the modified Morrison equation (Equation 1) yields an estimate of the apparent Ki value. For a competitive inhibitor, the Ki^{app} can be obtained from the y-intercept extracted from a linear regression analysis of a plot of K_{i,}^{aPP} versus substrate concentration.

$$v = A \cdot \frac{( [ E ] - [ I ] - {K}_{i}^{opp} ) + \sqrt{{( [ E ] - [ I ] {-}_{i}^{opp} )}^{2} + 4 [ E ] \cdot {K}_{i}^{opp}}}{2} \qquad ( Equation 1 )$$

Where A is equivalent to vₒ/E, the initial velocity (vₒ) of the enzymatic reaction in the absence of inhibitor (1) divided by the total enzyme concentration (E). In some embodiments, the anti-Galectin-9 antibody described herein has a Ki^{app} value of 1000, 900, 800, 700, 600, 500, 400, 300, 200, 100, 50, 40, 30, 20, 19, 18, 17, 16, 15, 14, 13, 12, 11, 10, 9, 8, 7, 6, 5 pM or less for the target antigen or antigen epitope. In some embodiments, the anti-Galectin-9 antibody has a lower Ki^{app} for a first target (e.g., the CRD2 of Galectin-9) relative to a second target (e.g., CRD1 of the Galectin-9). Differences in Ki^{app} (e.g., for specificity or other comparisons) can be at least 1.5, 2, 3, 4, 5, 10, 15, 20, 37.5, 50, 70, 80, 91, 100, 500, 1000, 10,000 or 10⁵ fold. In some examples, the anti-Galectin-9 antibody inhibits a first antigen (e.g., a first protein in a first conformation or mimic thereof) greater relative to a second antigen (e.g., the same first protein in a second conformation or mimic thereof; or a second protein). In some embodiments, any of the anti-Galectin-9 antibodies is further affinity matured to reduce the Ki^{app} of the antibody to the target antigen or antigenic epitope thereof.

In some embodiments, the anti-Galectin-9 antibody suppresses Dectin-1 signaling, e.g., in tumor infiltrating immune cells, such as macrophages. In some embodiments, the anti-Galectin-9 antibody suppresses Dectin-1 signaling triggered by Galectin-9 by at least 30% (e.g., 31%, 35%, 40%, 50%, 60%, 70%, 80%, 90%, 95% or greater, including any increment therein). Such inhibitory activity can be determined by conventional methods, such as routine assays. Alternatively or in addition, the anti-Galectin-9 antibody suppresses the T cell immunoglobulin mucin-3 (TIM-3) signaling initiated by Galectin-9. In some embodiments, the anti-Galectin-9 antibody suppresses the T cell immunoglobulin mucin-3 (TIM-3) signaling, e.g., in tumor infiltrating immune cells, e.g., in some embodiments, by at least 30% (e.g., 31%, 35%, 40%, 50%, 60%, 70%, 80%, 90%, 95% or greater, including any increment therein). Such inhibitory activity can be determined by conventional methods, such as routine assays.

In some embodiments, the anti-Galectin-9 antibody suppresses the CD206 signaling, e.g., in tumor infiltrating immune cells. In some embodiments, the anti-Galectin-9 antibody suppresses the CD206 signaling triggered by Galectin-9 by at least 30% (e.g., 31%, 35%, 40%, 50%, 60%, 70%, 80%, 90%, 95% or greater, including any increment therein). Such inhibitory activity can be determined by conventional methods, such as routine assays. In some embodiments, the anti-Galectin-9 antibody blocks or prevents binding of Galectin-9 to CD206 by at least 30% (e.g., 31%, 35%, 40%, 50%, 60%, 70%, 80%, 90%, 95% or greater, including any increment therein). Such inhibitory activity can be determined by conventional methods, such as routine assays.

In some embodiments, the anti-Galectin-9 antibody induces cell cytotoxicity, such as ADCC, in target cells expressing Galectin-9, e.g., wherein the target cells are cancer cells or immune suppressive immune cells. In some embodiments, the anti-Galectin-9 antibody induces apoptosis in immune cells, such as T cells, or cancer cells by at least 30% (e.g., 31%, 35%, 40%, 50%, 60%, 70%, 80%, 90%, 95% or greater, including any increment therein). Such inhibitory activity can be determined by conventional methods, such as routine assays. In some embodiments, any of the anti-Galectin-9 antibodies described herein induce cell cytotoxicity such as complement-dependent cytotoxicity (CDC) against target cells expressing Galectin-9.

Antibody-dependent cell-mediated phagocytosis (ADCP) is an important mechanism of action for antibodies that mediate part or all of their action though phagocytosis. In that case, antibodies mediate uptake of specific antigens by antigen presenting cells. ADCP can be mediated by monocytes, macrophages, neutrophils, and dendritic cells, through FcγRIIa, FcγRI, and FcγRIIIa, of which FcγRIIa (CD32a) on macrophages represent the predominant pathway.

In some embodiments, the anti-Galectin-9 antibody induces cell phagocytosis of target cells, e.g., cancer cells or immune suppressive immune cells expressing Galectin-9 (ADCP). In some embodiments, the anti-Galectin-9 antibody increases phagocytosis of target cells, e.g., cancer cells or immune suppressive immune cells, by at least 30% (e.g., 31%, 35%, 40%, 50%, 60%, 70%, 80%, 90%, 95% or greater, including any increment therein).

In some embodiments, the anti-Galectin-9 antibody described herein induces cell cytotoxicity such as complement-dependent cytotoxicity (CDC) against target cells, e.g., cancer cells or immune suppressive immune cells. In some embodiments, the anti-Galectin-9 antibody increases CDC against target cells by at least 30% (e.g., 31%, 35%, 40%, 50%, 60%, 70%, 80%, 90%, 95% or greater, including any increment therein).

In some embodiments, the anti-Galectin-9 antibody induces T cell activation, e.g., in tumor infiltrating T cells, i.e., suppress Galectin-9 mediated inhibition of T cell activation, either directly or indirectly. In some embodiments, the anti-Galectin-9 antibody promotes T cell activation by at least 30% (e.g., 31%, 35%, 40%, 50%, 60%, 70%, 80%, 90%, 95% or greater, including any increment therein). T cell activation can be determined by conventional methods, such as using well-known assays for measuring cytokines and checkpoint inhibitors (e.g., measurement of CD44, TNF alpha, IFNgamma, and/or PD-1). In some embodiments, the anti-Galectin-9 antibody promotes CD4+ cell activation by at least 30% (e.g., 31%, 35%, 40%, 50%, 60%, 70%, 80%, 90%, 95% or greater, including any increment therein). In a non-limiting example, the anti-Galectin antibody induces CD44 expression in CD4+ cells. In some embodiments, the anti-Galectin-9 antibody increases CD44 expression in CD4+ cells by at least 30% (e.g., 31%, 35%, 40%, 50%, 60%, 70%, 80%, 90%, 95% or greater, including any increment therein). In a non-limiting example, the anti-Galectin antibody induces IFNgamma expression in CD4+ cells. In some embodiments, the anti-Galectin-9 antibody increases IFNgamma expression in CD4+ cells by at least 30% (e.g., 31%, 35%, 40%, 50%, 60%, 70%, 80%, 90%, 95% or greater, including any increment therein). In a non-limiting example, the anti-Galectin antibody induces TNFalpha expression in CD4+ cells. In some embodiments, the anti-Galectin-9 antibody increases TNFalpha expression in CD4+ cells by at least 30% (e.g., 31%, 35%, 40%, 50%, 60%, 70%, 80%, 90%, 95% or greater, including any increment therein).

In some embodiments, the anti-Galectin-9 antibody promotes CD8+ cell activation by at least 30% (e.g., 31%, 35%, 40%, 50%, 60%, 70%, 80%, 90%, 95% or greater), including any increment therein). In a non-limiting example, the anti-Galectin antibody induces CD44 expression in CD8+ cells. In some embodiments, the anti-Galectin-9 antibody increases CD44 expression in CD8+ cells by at least 30% (e.g., 31%, 35%, 40%, 50%, 60%, 70%, 80%, 90%, 95% or greater, including any increment therein). In a non-limiting example, the anti-Galectin antibody induces IFNgamma expression in CD8+ cells. In some embodiments, the anti-Galectin-9 antibody increases IFNgamma expression in CD8+ cells by at least 30% (e.g., 31%, 35%, 40%, 50%, 60%, 70%, 80%, 90%, 95% or greater, including any increment therein). In a non-limiting example, the anti-Galectin antibody induces TNFalpha expression in CD8+ cells. In some embodiments, the anti-Galectin-9 antibody increases TNFalpha expression in CD8+ cells by at least 30% (e.g., 31%, 35%, 40%, 50%, 60%, 70%, 80%, 90%, 95% or greater, including any increment therein).

In some embodiments, an anti-Galectin-9 antibody as described herein has a suitable binding affinity for the target antigen (e.g., Galectin-9) or antigenic epitopes thereof. As used herein, “binding affinity” refers to the apparent association constant or K_{A}. The K_{A} is the reciprocal of the dissociation constant (K_{D}). The anti-Galectin-9 antibody described herein may have a binding affinity (K_{D}) of at least 10⁻⁵, 10⁻⁶, 10⁻⁷, 10⁻⁸, 10⁻⁹, 10⁻¹⁰ M, or lower for the target antigen or antigenic epitope. An increased binding affinity corresponds to a decreased K_{D}.

Binding affinity (or binding specificity) can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH7.4, 150 mM NaCl, 0.005% (v/v) Surfactant P20).

These techniques can be used to measure the concentration of bound binding protein as a function of target protein concentration. Under certain conditions, the fractional concentration of bound binding protein ([Bound]/[Total]) is generally related to the concentration of total target protein ([Target]) by the following equation:

$$[ Bound ] / [ Total ] = [ Target ] / ( Kd + [ Target ] )$$

It is not always necessary to make an exact determination of K_{A}, though, since sometimes it is sufficient to obtain a quantitative measurement of affinity, e.g., determined using a method such as ELISA or FACS analysis, is proportional to KA, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay. In some cases, the in vitro binding assay is indicative of in vivo activity. In other cases, the in vitro binding assay is not necessarily indicative of in vivo activity. In some cases, tight binding is beneficial, but in other cases tight binding is not as desirable in vivo, and an antibody with lower binding affinity is more desirable.

In some embodiments, the heavy chain of any of any of the anti-Galectin-9 antibodies as described herein further comprise a heavy chain constant region (CH) or a portion thereof (e.g., CH1, CH2, CH3, or a combination thereof). The heavy chain constant region can be of any suitable origin, e.g., human, mouse, rat, or rabbit. In one specific example, the heavy chain constant region is from a human IgG (a gamma heavy chain) of any IgG subfamily as described herein.

In some embodiments, the heavy chain constant region of the antibodies described herein comprise a single domain (e.g., CH1, CH2, or CH3) or a combination of any of the single domains, of a constant region (e.g., SEQ ID NO: 4, 5, 6). In some embodiments, the light chain constant region of the antibodies described herein comprise a single domain (e.g., CL), of a constant region. Exemplary light and heavy chain sequences are listed below. Exemplary light and heavy chain sequences are listed below. The hIgG1 LALA sequence includes two mutations, L234A and L235A (EU numbering), which suppress FcgR binding as well as a P329G mutation (EU numbering) to abolish complement C1q binding, thus abolishing all immune effector functions. The hIgG4 Fab Arm Exchange Mutant sequence includes a mutation to suppress Fab Arm Exchange (S228P; EU numbering). An IL2 signal sequence (MYRMQLLSCIALSLALVTNS; SEQ ID NO: 9) can be located N-terminally of the variable region. It is used in expression vectors, which is cleaved during secretion and thus not in the mature antibody molecule. The mature protein (after secretion) starts with “EVQ” for the heavy chain and “DIM” for the light chain. Amino acid sequences of exemplary heavy chain constant regions are provided below:

hIgG1 Heavy Chain Constant Region
(SEQ ID NO: 10)
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELLGGPSVFLFPPKPKDTLMISRTP
EVTCVVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK*
hIgG1 LALA Heavy Chain Constant Region
(SEQ ID NO: 12)
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPEAAGGPSVFLFPPKPKDTLMISRTP
EVTCVVVDVSHEDPEVKFNWYVDGVEVHNAKTKPREEQYNSTYRVVSVLTVLHQDWLNGKEYKCKVSNKA
LGAPIEKTISKAKGQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK*
hIgG4 Heavy Chain Constant Region
(SEQ ID NO: 13)
ASTKGPSVFPLAPCSRSTSESTAALGCLVKDYFPEPVTVSWNSGALTSGVHTFPAVLQSSGLYSLSSVVT
VPSSSLGTKTYTCNVDHKPSNTKVDKRVESKYGPPCPSCPAPEFLGGPSVFLFPPKPKDTLMISRTPEVT
CVVVDVSQEDPEVQFNWYVDGVEVHNAKTKPREEQFNSTYRVVSVLTVLHQDWLNGKEYKCKVSNKGLPS
SIEKTISKAKGQPREPQVYTLPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSPGK*
hIgG4 Heavy Chain Constant Region
(SEQ ID NO: 20)
ASTKGPSVFPLAPCSRSTSESTAALGCLVKDYFPEPVTVSWNSGALISGVHTFPAVLQSSGLYSLSSVVT
VPSSSLGTKTYTCNVDHKPSNTKVDKRVESKYGPPCPSCPAPEFLGGPSVFLFPPKPKDTLMISRTPEVT
CVVVDVSQEDPEVQFNWYVDGVEVHNAKTKPREEQFNSTYRVVSVLTVLHQDWLNGKEYKCKVSNKGLPS
SIEKTISKAKGQPREPQVYTLPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK*
hIgG4 mut Heavy Chain Constant Region
(SEQ ID NO: 14)
ASTKGPSVFPLAPCSRSTSESTAALGCLVKDYFPEPVTVSWNSGALTSGVHTFPAVLQSSGLYSLSSVVT
VPSSSLGTKTYTCNVDHKPSNTKVDKRVESKYGPPCPPCPAPEFLGGPSVFLFPPKPKDTLMISRTPEVT
CVVVDVSQEDPEVQFNWYVDGVEVHNAKTKPREEQFNSTYRVVSVLTVLHQDWLNGKEYKCKVSNKGLPS
SIEKTISKAKGQPREPQVYTLPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSPGK*
hIG4 mut Heavy Chain Constant Region
(SEQ ID NO: 21)
ASTKGPSVFPLAPCSRSTSESTAALGCLVKDYFPEPVTVSWNSGALTSGVHTEPAVLQSSGLYSLSSVVT
VPSSSLGTKTYTCNVDHKPSNTKVDKRVESKYGPPCPPCPAPEFLGGPSVFLFPPKPKDTLMISRTPEVT
CVVVDVSQEDPEVQFNWYVDGVEVHNAKTKPREEQFNSTYRVVSVLTVLHQDWLNGKEYKCKVSNKGLPS
SIEKTISKAKGQPREPQVYTLPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK*

In some embodiments, anti-Galectin-9 antibodies having any of the above heavy chain constant regions are paired with a light chain having the following light chain constant region:

Light Chain Constant Region
(SEQ ID NO: 11)
TVAAPSVEIFPPSDEQLKSGTASVVCLLNNFYPREAKVQWKVDNALQSG
NSQESVTEQDSKDSTYSLSSTLTLSKADYEKHKVYACEVTHQGLSSPVT
KSFNRGEC

Exemplary full length anti-Galectin-9 antibodies are provided below:

G9.2-17 hIgG1 Heavy Chain
(SEQ ID NO: 16)
EVQLVESGGGLVQPGGSLRLSCAASGFTVSSSSIHWVRQAPGKGLEWVAYISSSSGYTYYADSVKGRFTI
SADTSKNTAYLQMNSLRAEDTAVYYCARYWSYPSWWPYRGMDYWGQGTLVTVSSASTKGPSVFPLAPSSK
STSGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYKTTPPVLDSDGSFFLYSKLIVD
KSRWQQGNVFSCSVMHEALHNHYTQKSLSLSPGK*
G9.2-17 hIgG1 LALA Heavy Chain
(SEQ ID NO: 17)
EVQLVESGGGLVQPGGSLRLSCAASGFTVSSSSIHWVRQAPGKGLEWVAYISSSSGYTYYADSVKGRFTI
SADTSKNTAYLQMNSLRAEDTAVYYCARYWSYPSWWPYRGMDYWGQGTLVTVSSASTKGPSVFPLAPSSK
STSGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPEAAGGPSVFLFPPKPKDTLMISRTPEVTCVVVDVSHEDPEV
KFNWYVDGVEVHNAKTKPREEQYNSTYRVVSVLTVLHQDWLNGKEYKCKVSNKALGAPIEKTISKAKGQP
REPQVYTLPPSREEMTKNQVSLTCLVKGFYPSDIAVEWESNGQPENNYKTTPPVLDSDGSFFLYSKLTVD
KSRWQQGNVFSCSVMHEALHNHYTQKSLSLSPGK*
G9.2-17 hIgG4 Heavy Chain
(SEQ ID NO: 18)
EVQLVESGGGLVQPGGSLRLSCAASGFTVSSSSIHWVRQAPGKGLEWVAYISSSSGYTYYADSVKGRFTI
SADTSKNTAYLQMNSLRAEDTAVYYCARYWSYPSWWPYRGMDYWGQGTLVTVSSASTKGPSVEPLAPCSR
STSESTAALGCLVKDYFPEPVTVSWNSGALTSGVHTFPAVLQSSGLYSLSSVVTVPSSSLGTKTYTCNVD
HKPSNTKVDKRVESKYGPPCPSCPAPEFLGGPSVFLFPPKPKDTLMISRTPEVTCVVVDVSQEDPEVQFN
WYVDGVEVHNAKTKPREEQFNSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPPVLDSDGSFFLYSRLTVDKSR
WQEGNVFSCSVMHEALHNHYTQKSLSLSPGK*
G9.2-17 hIgG4 Heavy Chain
(SEQ ID NO: 22)
EVQLVESGGGLVQPGGSLRLSCAASGFTVSSSSIHWVRQAPGKGLEWVAYISSSSGYTYYADSVKGRFTI
SADTSKNTAYLQMNSLRAEDTAVYYCARYWSYPSWWPYRGMDYWGQGTLVTVSSASTKGPSVFPLAPCSR
STSESTAALGCLVKDYFPEPVTVSWNSGALTSGVHTFPAVLQSSGLYSLSSVVTVPSSSLGTKTYTCNVD
HKPSNTKVDKRVESKYGPPCPSCPAPEFLGGPSVFLFPPKPKDTLMISRTPEVTCVVVDVSQEDPEVQFN
WYVDGVEVHNAKTKPREEQFNSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPPVLDSDGSFFLYSRLTVDKSR
WQEGNVFSCSVMHEALHNHYTQKSLSLSLGK*
G9.2-17 hIgG4 Fab Arm Exchange mut Heavy Chain
(SEQ ID NO: 19)
EVQLVESGGGLVQPGGSLRLSCAASGFTVSSSSIHWVRQAPGKGLEWVAYISSSSGYTYYADSVKGRFTI
SADTSKNTAYLQMNSLRAEDTAVYYCARYWSYPSWWPYRGMDYWGQGTLVTVSSASTKGPSVEPLAPCSR
STSESTAALGCLVKDYFPEPVTVSWNSGALISGVHTFPAVLQSSGLYSLSSVVTVPSSSLGTKTYTCNVD
HKPSNTKVDKRVESKYGPPCPPCPAPEFLGGPSVFLFPPKPKDTLMISRTPEVTCVVVDVSQEDPEVQFN
WYVDGVEVHNAKTKPREEQFNSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPPVLDSDGSFFLYSRLTVDKSR
WQEGNVFSCSVMHEALHNHYTQKSLSLSPGK*
G9.2-17 hIgG4 Fab Arm Exchange mut Heavy Chain
(SEQ ID NO: 23)
EVQLVESGGGLVQPGGSLRLSCAASGFTVSSSSIHWVRQAPGKGLEWVAYISSSSGYTYYADSVKGRFTI
SADTSKNTAYLQMNSLRAEDTAVYYCARYWSYPSWWPYRGMDYWGQGTLVTVSSASTKGPSVEPLAPCSR
STSESTAALGCLVKDYFPEPVTVSWNSGALISGVHTFPAVLQSSGLYSLSSVVTVPSSSLGTKTYTCNVD
HKPSNTKVDKRVESKYGPPCPPCPAPEFLGGPSVFLFPPKPKDTLMISRTPEVTCVVVDVSQEDPEVQFN
WYVDGVEVHNAKTKPREEQFNSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPPVLDSDGSFFLYSRLTVDKSR
WQEGNVFSCSVMHEALHNHYTQKSLSLSLGK*

Any of the above heavy chain can be paired with a Light Chain of (SEQ ID NO: 15) shown below:

DIQMTQSPSSLSASVGDRVTITCRASQSVSSAVAWYQQKPGKAPKLLIY
SASSLYSGVPSRFSGSRSGTDFTLTISSLQPEDFATYYCQQSSTDPITF
GQGTKVEIKRTVAAPSVEIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC*

In some embodiments, the anti-Galectin-9 antibody comprises a heavy chain IgG1 constant region that has at least 80% (e.g., 80%, 85%, 90%, 91%, 92%, 93%, 94%, 95%, 96%, 97%, 98%, or 99% and any increment therein) sequence identity to SEQ ID NO: 10. In one embodiment, the constant region of the anti-Galectin-9 antibody comprises a heavy chain IgG4 constant region comprising SEQ ID NO: 10. In one embodiment, the constant region of the anti-Galectin-9 antibody comprises a heavy chain IgG1 constant region consisting of SEQ ID NO: 10.

In some embodiments, the anti-Galectin-9 antibody comprises a heavy chain IgG4 constant region that has at least 80% (e.g., 80%, 85%, 90%, 91%, 92%, 93%, 94%, 95%, 96%, 97%, 98%, or 99% and any increment therein) sequence identity to SEQ ID NO: 20. In one embodiment, the constant region of the anti-Galectin-9 antibody comprises a heavy chain IgG4 constant region comprising SEQ ID NO: 20. In one embodiment, the constant region of the anti-Galectin-9 antibody comprises a heavy chain IgG4 constant region consisting of SEQ ID NO: 20.

In some embodiments, the constant region is from human IgG4. In one embodiment, the anti-Galectin-9 antibody comprises a heavy chain IgG4 constant region that has at least 80% (e.g., 80%, 85%, 90%, 91%, 92%, 93%, 94%, 95%, 96%, 97%, 98%, or 99% and any increment therein) sequence identity to SEQ ID NO: 13. In one embodiment, the anti-Galectin-9 antibody comprises a heavy chain IgG4 constant region comprising SEQ ID NO: 13. In one embodiment, the anti-Galectin-9 antibody comprises a heavy chain IgG4 constant region consisting of SEQ ID NO: 13.

In some embodiments, the constant region is from human IgG4. In one embodiment, the anti-Galectin-9 antibody comprises a heavy chain IgG4 constant region that has at least 80% (e.g., 80%, 85%, 90%, 91%, 92%, 93%, 94%, 95%, 96%, 97%, 98%, or 99% and any increment therein) sequence identity to SEQ ID NO: 20. In one embodiment, the anti-Galectin-9 antibody comprises a heavy chain IgG4 constant region comprising SEQ ID NO: 20. In one embodiment, the anti-Galectin-9 antibody comprises a heavy chain IgG4 constant region consisting of SEQ ID NO: 20.

In any of these embodiments, the anti-Galectin-9 antibody comprises a light chain constant region that has at least 80% (e.g., 80%, 85%, 90%, 91%, 92%, 93%, 94%, 95%, 96%, 97%, 98%, or 99% and any increment therein) sequence identity to SEQ ID NO: 11. In some embodiments, the anti-Galectin-9 antibody comprises a light chain constant region comprising SEQ ID NO: 11. In some embodiments, the anti-Galectin-9 antibody comprises a light chain constant region consisting of SEQ ID NO: 11.

In some embodiments, the IgG is a mutant with minimal Fc receptor engagement. In one example, the constant region is from a human IgG1 LALA. In one embodiment, the anti-Galectin-9 antibody comprises a heavy chain IgG1 constant region that has at least 80% (e.g., 80%, 85%, 90%, 91%, 92%, 93%, 94%, 95%, 96%, 97%, 98%, or 99% and any increment therein) sequence identity to SEQ ID NO: 12. In one embodiment, the anti-Galectin-9 antibody comprises a heavy chain IgG1 constant region comprising SEQ ID NO: 12. In one embodiment, the anti-Galectin-9 antibody comprises a heavy chain IgG1 constant region consisting of SEQ ID NO: 12.

In some embodiments, the anti-Galectin-9 antibody comprises a modified constant region. In some embodiments, the anti-Galectin-9 antibody comprise a modified constant region that is immunologically inert, e.g., does not trigger complement mediated lysis, or does not stimulate antibody-dependent cell mediated cytotoxicity (ADCC). ADCC activity can be assessed using methods disclosed in U.S. Pat. No. 5,500,362. In other embodiments, the constant region is modified as described in Eur. J. Immunol. (1999) 29:2613-2624; PCT Application No. PCT/GB99/01441; and/or UK Patent Application No. 9809951.8. In some embodiments, the IgG4 constant region is a mutant with reduced heavy chain exchange. In some embodiments, the constant region is from a human IgG4 Fab Arm Exchange mutant S228P.

In one embodiment, the constant region of the anti-Galectin-9 antibody comprises a heavy chain IgG4 constant region that has at least 80% (e.g., 80%, 85%, 90%, 91%, 92%, 93%, 94%, 95%, 96%, 97%, 98%, or 99% and any increment therein) sequence identity to SEQ ID NO: 14. In one embodiment, the constant region of the anti-Galectin-9 antibody comprises a heavy chain IgG4 constant region comprising SEQ ID NO: 14. In one embodiment, the constant region of the anti-Galectin-9 antibody comprises a heavy chain IgG4 constant region consisting of SEQ ID NO: 14.

In one embodiment, the anti-Galectin-9 antibody comprises a heavy chain IgG4 constant region that has at least 80% (e.g., 80%, 85%, 90%, 91%, 92%, 93%, 94%, 95%, 96%, 97%, 98%, or 99% and any increment therein) sequence identity to SEQ ID NO: 21. In one embodiment, the anti-Galectin-9 antibody comprises a heavy chain IgG4 constant region comprising SEQ ID NO: 21. In one embodiment, the anti-Galectin-9 antibody comprises a heavy chain IgG4 constant region consisting of SEQ ID NO: 21.

In some embodiments, the anti-Galectin-9 antibody has chains corresponding to SEQ ID NO: 15 for the light chains; and the amino acid sequences of exemplary heavy chains correspond to SEQ ID NO: 10 (hIgG1); 12 (hIgG1 LALA); 13 (hIgG4); 20 (hIgG4); 14 (hIgG4 mut); and 21 (hIgG4 mut).

In some embodiments, the anti-Galectin-9 antibody has a light chain comprising, consisting essentially of, or consisting of SEQ ID NO: 15. In some embodiments, the anti-Galectin-9 antibody has a heavy chain comprising, consisting essentially of, or consisting of any one of the sequences selected from the group consisting of SEQ ID NO: 16-19, 22 and 23. In some embodiments, the anti-Galectin-9 antibody has a light chain comprising, consisting essentially of, or consisting of SEQ ID NO: 15 and a heavy chain comprising, consisting essentially of, or consisting of any one of the sequences selected from the group consisting of SEQ ID NO: 16-19. In some embodiments, the anti-Galectin-9 antibody has a light chain comprising SEQ ID NO: 15 and a heavy chain comprising any one of the sequences selected from the group consisting of SEQ ID NO: 16-19, 22 and 23. In some embodiments, the anti-Galectin-9 antibody has a light chain consisting essentially of SEQ ID NO: 15 and a heavy chain consisting essentially of any one of the sequences selected from the group consisting of SEQ ID NO: 16-19, 22 and 23. In some embodiments, the anti-Galectin-9 antibody has a light chain consisting of SEQ ID NO: 15 and a heavy chain consisting of any one of the sequences selected from the group consisting of SEQ ID NO: 16-19, 22 and 23. In one specific embodiment, the anti-Galectin-9 antibody has a light chain consisting essentially of SEQ ID NO: 15 and a heavy chain consisting essentially of SEQ ID NO: 19. In another specific embodiment, the anti-Galectin-9 antibody has a light chain consisting essentially of SEQ ID NO: 15 and a heavy chain consisting essentially of SEQ ID NO: 20.

In one embodiment, the anti-Galectin-9 antibody comprises a heavy chain sequence having at least 80% (e.g., 80%, 85%, 90%, 91%, 92%, 93%, 94%, 95%, 96%, 97%, 98%, or 99% and any increment therein) sequence identity to SEQ ID NO: 16. In one embodiment, the anti-Galectin-9 antibody comprises a heavy chain sequence comprising SEQ ID NO: 16. In one embodiment, the anti-Galectin-9 antibody comprises a heavy chain sequence consisting of SEQ ID NO: 16.

In one embodiment, the anti-Galectin-9 antibody comprises a heavy chain sequence having at least 80% (e.g., 80%, 85%, 90%, 91%, 92%, 93%, 94%, 95%, 96%, 97%, 98%, or 99% and any increment therein) sequence identity to SEQ ID NO: 17. In one embodiment, the anti-Galectin-9 antibody comprises a heavy chain sequence comprising SEQ ID NO: 17. In one embodiment, the anti-Galectin-9 antibody comprises a heavy chain sequence consisting of SEQ ID NO: 17.

In one embodiment, the anti-Galectin-9 antibody comprises a heavy chain sequence having at least 80% (e.g., 80%, 85%, 90%, 91%, 92%, 93%, 94%, 95%, 96%, 97%, 98%, or 99% and any increment therein) sequence identity to SEQ ID NO: 18. In one embodiment, the anti-Galectin-9 antibody comprises a heavy chain sequence comprising SEQ ID NO: 18. In one embodiment, the anti-Galectin-9 antibody comprises a heavy chain sequence consisting of SEQ ID NO: 18.

In one embodiment, the anti-Galectin-9 antibody comprises a heavy chain sequence having at least 80% (e.g., 80%, 85%, 90%, 91%, 92%, 93%, 94%, 95%, 96%, 97%, 98%, or 99% and any increment therein) sequence identity to SEQ ID NO: 22. In one embodiment, the anti-Galectin-9 antibody comprises a heavy chain sequence comprising SEQ ID NO: 22. In one embodiment, the anti-Galectin-9 antibody comprises a heavy chain sequence consisting of SEQ ID NO: 22.

In one embodiment, the anti-Galectin-9 antibody comprises a heavy chain sequence having at least 80% (e.g., 80%, 85%, 90%, 91%, 92%, 93%, 94%, 95%, 96%, 97%, 98%, or 99% and any increment therein) sequence identity to SEQ ID NO: 19. In one embodiment, the anti-Galectin-9 antibody comprises a heavy chain sequence comprising SEQ ID NO: 19. In one embodiment, the anti-Galectin-9 antibody comprises a heavy chain sequence consisting of SEQ ID NO: 19.

In one embodiment, the anti-Galectin-9 antibody comprises a heavy chain sequence having at least 80% (e.g., 80%, 85%, 90%, 91%, 92%, 93%, 94%, 95%, 96%, 97%, 98%, or 99% and any increment therein) sequence identity to SEQ ID NO: 23. In one embodiment, the anti-Galectin-9 antibody comprises a heavy chain sequence comprising SEQ ID NO: 23. In one embodiment, the anti-Galectin-9 antibody comprises a heavy chain sequence consisting of SEQ ID NO: 23.

In any of these embodiments, the anti-Galectin-9 antibody comprises a light chain sequence having at least 80% (e.g., 80%, 85%, 90%, 91%, 92%, 93%, 94%, 95%, 96%, 97%, 98%, or 99% and any increment therein) sequence identity to SEQ ID NO: 15. In some embodiments, the anti-Galectin-9 antibody comprises a light chain sequence comprising SEQ ID NO: 15. In some embodiments, the anti-Galectin-9 antibody comprises a light chain sequence consisting of SEQ ID NO: 15.

In specific examples, the anti-Galectin-9 antibody used in the treatment methods disclosed herein has a heavy chain of SEQ ID NO:19 and a light chain of SEQ ID NO:15. In some embodiments, the the anti-Galectin-9 antibody used in the treatment methods disclosed herein is G9.2-17 IgG4.

In some embodiments, any of the anti-Galectin-9 antibody disclosed herein (e.g., G9.2-17(IgG4)) may have the C-terminus lysine residue of the heavy chain deleted.

### Preparation of Anti-Galectin-9 Antibodies

Antibodies capable of binding Galectin-9 as described herein can be made by any method known in the art, including but not limited to, recombinant technology. One example is provided below.

Nucleic acids encoding the heavy and light chain of an anti-Galectin-9 antibody as described herein can be cloned into one expression vector, each nucleotide sequence being in operable linkage to a suitable promoter. In one example, each of the nucleotide sequences encoding the heavy chain and light chain is in operable linkage to a distinct promoter. Alternatively, the nucleotide sequences encoding the heavy chain and the light chain can be in operable linkage with a single promoter, such that both heavy and light chains are expressed from the same promoter. When necessary, an internal ribosomal entry site (IRES) can be inserted between the heavy chain and light chain encoding sequences.

In some examples, the nucleotide sequences encoding the two chains of the antibody are cloned into two vectors, which can be introduced into the same or different cells. When the two chains are expressed in different cells, each of them can be isolated from the host cells expressing such and the isolated heavy chains and light chains can be mixed and incubated under suitable conditions allowing for the formation of the antibody.

Generally, a nucleic acid sequence encoding one or all chains of an antibody can be cloned into a suitable expression vector in operable linkage with a suitable promoter using methods known in the art. For example, the nucleotide sequence and vector can be contacted, under suitable conditions, with a restriction enzyme to create complementarity ends on each molecule that can pair with each other and be joined together with a ligase. Alternatively, synthetic nucleic acid linkers can be ligated to the termini of a gene. These synthetic linkers contain nucleic acid sequences that correspond to a particular restriction site in the vector. The selection of expression vectors/promoter would depend on the type of host cells for use in producing the antibodies.

A variety of promoters can be used for expression of the antibodies described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter.

Regulatable promoters can also be used. Such regulatable promoters include those using the lac repressor from E. coli as a transcription modulator to regulate transcription from lac operator-bearing mammalian cell promoters [Brown, M. et al., Cell, 49:603-612 (1987)], those using the tetracycline repressor (tetR) [Gossen, M., and Bujard, H., Proc. Natd. Acad. Sci. USA 89:5547-5551 (1992); Yao, F. et al., Human Gene Therapy, 9:1939-1950 (1998); Shockelt, P., et al., Proc. Natd. Acad. Sci. USA, 92:6522-6526 (1995)]. Other systems include FK506 dimer, VP16 or p65 using astradiol, RU486, diphenol murislerone, or rapamycin. Inducible systems are available from Invitrogen, Clontech and Ariad.

Regulatable promoters that include a repressor with the operon can be used. In one embodiment, the lac repressor from E. coli can function as a transcriptional modulator to regulate transcription from lac operator-bearing mammalian cell promoters (M. Brown et al., Cell, 49:603-612 (1987); Gossen and Bujard (1992); M. Gossen et al., Natd. Acad. Sci. USA, 89:5547-5551 (1992)) combined the tetracycline repressor (tetR) with the transcription activator (VP 16) to create a tetR-mammalian cell transcription activator fusion protein, tTa (tetR-VP 16), with the tetO-bearing minimal promoter derived from the human cytomegalovirus (hCMV) major immediate-early promoter to create a tetR-tet operator system to control gene expression in mammalian cells. In one embodiment, a tetracycline inducible switch is used. The tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter (Yao et al., Human Gene Therapy, 10(16):1392-1399 (2003)). One particular advantage of this tetracycline inducible switch is that it does not require the use of a tetracycline repressor-mammalian cells transactivator or repressor fusion protein, which in some instances can be toxic to cells (Gossen et al., Natd. Acad. Sci. USA, 89:5547-5551 (1992); Shockett et al., Proc. Natl. Acad. Sci. USA, 92:6522-6526 (1995)), to achieve its regulatable effects.

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art.

Examples of polyadenylation signals useful to practice the methods described herein include, but are not limited to, human collagen I polyadenylation signal, human collagen II polyadenylation signal, and SV40 polyadenylation signal.

One or more vectors (e.g., expression vectors) comprising nucleic acids encoding any of the antibodies may be introduced into suitable host cells for producing the antibodies. The host cells can be cultured under suitable conditions for expression of the antibody or any polypeptide chain thereof. Such antibodies or polypeptide chains thereof can be recovered by the cultured cells (e.g., from the cells or the culture supernatant) via a conventional method, e.g., affinity purification. If necessary, polypeptide chains of the antibody can be incubated under suitable conditions for a suitable period of time allowing for production of the antibody.

In some embodiments, methods for preparing an antibody described herein involve a recombinant expression vector that encodes both the heavy chain and the light chain of an anti-Galectin-9 antibody, as also described herein. The recombinant expression vector can be introduced into a suitable host cell (e.g., a dhfr-CHO cell) by a conventional method, e.g., calcium phosphate-mediated transfection. Positive transformant host cells can be selected and cultured under suitable conditions allowing for the expression of the two polypeptide chains that form the antibody, which can be recovered from the cells or from the culture medium. When necessary, the two chains recovered from the host cells can be incubated under suitable conditions allowing for the formation of the antibody.

In one example, two recombinant expression vectors are provided, one encoding the heavy chain of the anti-Galectin-9 antibody and the other encoding the light chain of the anti-Galectin-9 antibody. Both of the two recombinant expression vectors can be introduced into a suitable host cell (e.g., dhfr-CHO cell) by a conventional method, e.g., calcium phosphate-mediated transfection. Alternatively, each of the expression vectors can be introduced into a suitable host cell. Positive transformants can be selected and cultured under suitable conditions allowing for the expression of the polypeptide chains of the antibody. When the two expression vectors are introduced into the same host cells, the antibody produced therein can be recovered from the host cells or from the culture medium. If necessary, the polypeptide chains can be recovered from the host cells or from the culture medium and then incubated under suitable conditions allowing for formation of the antibody. When the two expression vectors are introduced into different host cells, each of them can be recovered from the corresponding host cells or from the corresponding culture media. The two polypeptide chains can then be incubated under suitable conditions for formation of the antibody.

Standard molecular biology techniques are used to prepare the recombinant expression vector, transfect the host cells, select for transformants, culture the host cells and recovery of the antibodies from the culture medium. For example, some antibodies can be isolated by affinity chromatography with a Protein A or Protein G coupled matrix.

Any of the nucleic acids encoding the heavy chain, the light chain, or both of an anti-Galectin-9 antibody as described herein, vectors (e.g., expression vectors) containing such; and host cells comprising the vectors are within the scope of the present disclosure.

Anti-Galectin-9 antibodies thus prepared can be characterized using methods known in the art, whereby reduction, amelioration, or neutralization of Galectin-9 biological activity is detected and/or measured. For example, in some embodiments, an ELISA-type assay is suitable for qualitative or quantitative measurement of Galectin-9 inhibition of Dectin-1 or TIM-3 signaling.

The bioactivity of an anti-Galectin-9 antibody can verified by incubating a candidate antibody with Dectin-1 and Galectin-9, and monitoring any one or more of the following characteristics: (a) binding between Dectin-1 and Galectin-9 and inhibition of the signaling transduction mediated by the binding; (b) preventing, ameliorating, or treating any aspect of a solid tumor; (c) blocking or decreasing Dectin-1 activation; (d) inhibiting (reducing) synthesis, production or release of Galectin-9. Alternatively, TIM-3 can be used to verify the bioactivity of an anti-Galectin-9 antibody using the protocol described above. Alternatively, CD206 can be used to verify the bioactivity of an anti-Galectin-9 antibody using the protocol described above.

In some embodiments, bioactivity or efficacy is assessed in a subject, e.g., by measuring peripheral and intra-tumoral T cell ratios, T cell activation, or by macrophage phenotyping.

Additional assays to determine bioactivity of an anti-Galectin-9 antibody include measurement of CD8+ and CD4+(conventional) T-cell activation (in an in vitro or in vivo assay, e.g., by measuring inflammatory cytokine levels, e.g., IFNgamma, TNFalpha, CD44, ICOS granzymeB, Perforin, IL2 (upregulation); CD26L and IL-10 (downregulation)); measurement of reprogramming of macrophages (in vitro or in vivo), e.g., from the M2 to the M1 phenotype (e.g., increased MHCII, reduced CD206, increased TNF-alpha and iNOS), Alternatively, levels of ADCC can be assessed, e.g., in an in vitro assay, as described herein.

### Methods of Treatment

The present disclosure provides methods for treating solid tumors including, but not limited to, head and neck cancer, urothelial carcinoma, gastric esophageal cancer, or non-small cell lung cancer, using any of the anti-Galectin antibodies, for example G9.2-17, e.g., G9.2-17 IgG4, either alone or in combination with a checkpoint inhibitor such as an anti-PD-1 antibody, for example, tislelizumab. Additional target solid tumors for treatment by the method disclosed herein may include pancreatic ductal adenocarcinoma (PDAC), colorectal cancer (CRC), hepatocellular carcinoma (HCC), cholangiocarcinoma, renal cell carcinoma, and breast cancer.

Conventional methods, known to those of ordinary skill in the art of medicine, can be used to administer the pharmaceutical composition to the subject, depending upon the type of disease to be treated or the site of the disease. In some embodiments, the anti-Galectin-9 antibody and/or the anti-PD-1 antibody can be administered to a subject by intravenous infusion.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous infusion, water soluble antibodies can be administered by the drip method, whereby a pharmaceutical formulation containing the antibody and a physiologically acceptable excipient is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, a symptom of the disease or disorder, or the predisposition toward the disease or disorder.

Alleviating a target disease/disorder includes delaying the development or progression of the disease or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

(i) Treatment with Anti-Galectin 9 Antibodies

Any of the anti-Galectin-9 antibodies described herein can be used in any of the methods described herein. In some embodiments, the anti-Galectin-9 antibody is G9.2-17, e.g., G9.2-17(IgG4). Such antibodies can be used for treating diseases associated with Galectin-9. In some aspects, the invention provides methods of treating cancer. In some embodiments, the present disclosure methods for reducing, ameliorating, or eliminating one or more symptom(s) associated with cancer.

In some embodiments, the anti-Galectin-9 antibody is an antibody having the same heavy chain CDR sequences and/or the same light chain CDR sequences as reference antibody G9.2-17. In some embodiments, the anti-Galectin-9 antibody is an antibody having the same VH and VL sequences as reference antibody G9.2-17. In some embodiments, such an antibody is an IgG1 molecule (e.g., having a wild-type IgG1 constant region or a mutant thereof as those disclosed herein). Alternatively, the antibody is an IgG4 molecule (e.g., having a wild-type IgG4 constant region or a mutant thereof as those described herein). In some embodiments, the antibody comprises a light chain complementarity determining region 1 (CDR1) set forth as SEQ ID NO: 1, a light chain complementarity determining region 2 (CDR2) set forth as SEQ ID NO: 2, and a light chain complementarity determining region 3 (CDR3) set forth as SEQ ID NO: 3 and/or comprises a heavy chain complementarity determining region 1 (CDR1) set forth as SEQ ID NO: 4, a heavy chain complementarity determining region 2 (CDR2) set forth as SEQ ID NO: 5, and a heavy chain complementarity determining region 3 (CDR3) set forth as SEQ ID NO: 6. In some embodiments, the antibody comprises a heavy chain variable region comprising SEQ ID NO: 7. In some embodiments, the antibody comprises a light chain variable region comprising SEQ ID NO: 8. In some embodiments, the antibody comprises a heavy chain variable region comprising SEQ ID NO: 7 and a light chain variable region comprising SEQ ID NO: 8. In some embodiments, the antibody comprises a heavy chain comprising SEQ ID NO: 19. In some embodiments, the antibody comprises a light chain comprising SEQ ID NO: 15. In specific examples, the anti-Galectin-9 antibody used herein (G9.2-17(IgG4)) has a heavy chain of SEQ ID NO:19 and a light chain of SEQ ID NO:15.

An effective amount of the anti-Galectin-9 antibody described herein (e.g., G9.2-17(IgG4)) can be administered to a subject (e.g., a human) in need of the treatment via a suitable route, systemically or locally. In some embodiments, the anti-Galectin-9 antibodies are administered by intravenous administration, e.g., as a bolus or by continuous infusion over a period of time, by intramuscular, intraperitoneal, intracerebrospinal, subcutaneous, intra-arterial, intra-articular, intrasynovial, intrathecal, intratumoral, sub-urothelial, oral, inhalation or topical routes. In one embodiment, the anti-Galectin-9 antibody is administered to the subject by intravenous infusion. In one embodiment, the anti-galectin-9 antibody is administered to the subject intraperitoneally.

As used herein, “an effective amount” refers to the amount of each active agent required to confer therapeutic effect on the subject, either alone or in combination with one or more other active agents. In some embodiments, the therapeutic effect is reduced Galectin-9 activity and/or amount/expression, reduced Dectin-1 signaling, reduced TIM-3 signaling, reduced CD206 signaling, or increased anti-tumor immune responses in the tumor microenvironment. Non-limiting examples of increased anti-tumor responses include increased activation levels of effector T cells or switching of the TAMs from the M2 to the M1 phenotype. In some cases, the anti-tumor response includes increased ADCC responses. Determination of whether an amount of the antibody achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally contribute to the determination of the dosage. For example, antibodies that are compatible with the human immune system, such as humanized antibodies or fully human antibodies, are in some instances used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of a target disease/disorder. Alternatively, sustained continuous release formulations of an antibody may be appropriate. Various formulations and devices for achieving sustained release are known in the art.

In one example, dosages for an antibody as described herein are determined empirically in individuals who have been given one or more administration(s) of the antibody. Individuals are given incremental dosages of the antagonist. To assess efficacy of the antagonist, an indicator of the disease/disorder can be followed.

In some embodiments, the antibodies described herein, e.g., G9.2-17 such as G9.2-17(IgG4), are administered to a subject in need of the treatment at an amount sufficient to inhibit the activity of Galectin-9 (and/or Dectin-1 or TIM-3 or CD206) in immune suppressive immune cells in a tumor by at least 20% (e.g., 30%, 40%, 50%, 60%, 70%, 80%, 90% or greater) in vivo. In other embodiments, the antibodies described herein, e.g, G9.2-17, are administered in an amount effective in reducing the activity level of Galectin-9 (and/or Dectin-1 or TIM-3 or CD206) in immune suppressive immune cells in a tumor by at least 20% (e.g., 30%, 40%, 50%, 60%, 70%, 80%, 90% or greater) (as compared to levels prior to treatment or in a control subject). In some embodiments, the antibodies described herein, e.g., G9.2-17, are administered to a subject in need of the treatment at an amount sufficient to promote M1-like programming in TAMs by at least 20% (e.g., 30%, 40%, 50%, 60%, 70%, 80%, 90% or greater) in vivo (as compared to levels prior to treatment or in a control subject).

The term “about” or “approximately” means within an acceptable error range for the particular value as determined by one of ordinary skill in the art, which are dependent in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

In some embodiments, the antibody is G9.2-17 IgG4. In some embodiments, the anti-Galectin-9 antibody is administered to the subject at a dose of about 0.2 mg/kg to about 32 mg/kg, e.g., 0.2 mg/kg, 0.63 mg/kg, 2 mg/kg, 4 mg/kg, 6 mg/kg, 6.3 mg/kg, 8 mg/kg, 10 mg/kg, 12 mg/kg, and 16 mg/kg or a higher dose level. In some embodiments, the anti-Galectin-9 antibody is administered to the subject at a dose of about 1 mg/kg to about 32 mg/kg, e.g., the dose may be selected from 2 mg/kg, 4 mg/kg, 8 mg/kg, 12 mg/kg, and 16 mg/kg or a higher dose level. In some embodiments, the anti-Galectin-9 antibody is administered to the subject at a dose of about 0.2 mg/kg to about 32 mg/kg, e.g., the dose may be selected from 0.2 mg/kg, 0.63 mg/kg, 2 mg/kg, 4 mg/kg, 6 mg/kg, 6.3 mg/kg, 10 mg/kg, and 16 mg/kg or a higher dose level. In some embodiments, the antibody is administered once every two weeks, e.g., via intravenous infusion.

In some embodiments, the anti-Galectin 9 antibody disclosed herein (e.g., G9.2-17 IgG4) is administered via a 30 minute to 6-hour infusion intravenously. In some examples the intravenous infusion of the anti-Galectin 9 antibody may be performed for 30 minutes to 2 hours. In other examples, the the anti-Galectin 9 antibody may be administered via a long infusion period, for example, about 2-6 hours, e.g., about 2-4 hours or about 4-6 hours. In specific examples, examples anti-Galectin 9 antibody may be infused intravenous in a period of about 3 hours, about 4 hours, about 5 hours, or about 6 hours.

In some embodiments, the anti-Galectin-9 antibody for use in any of the methods disclosed herein (e.g., G9.2-17(IgG4) as disclosed herein) may be administered to the subject at a dose of about 0.2 mg/kg to about 32 mg/kg, e.g., the dose may be selected from 0.2 mg/kg, 0.63 mg/kg, 2 mg/kg, 4 mg/kg, 6 mg/kg, 6.3 mg/kg, 8 mg/kg, 10 mg/kg, 12 mg/kg, and 16 mg/kg or a higher dose level. In some embodiments, the anti-Galectin-9 antibody is administered to the subject at a dose of about 1 mg/kg to about 32 mg/kg, e.g., the dose may be selected from 2 mg/kg, 4 mg/kg, 8 mg/kg, 12 mg/kg, and 16 mg/kg or a higher dose level. In some embodiments, the anti-Galectin-9 antibody is administered to the subject at a dose of about 0.2 mg/kg to about 32 mg/kg, e.g., the dose may be selected from 0.2 mg/kg, 0.63 mg/kg, 2 mg/kg, 4 mg/kg, 6 mg/kg, 6.3 mg/kg, 10 mg/kg, or 16 mg/kg or a higher dose level.

In some embodiments, the anti-Galectin-9 antibody (e.g., G9.2-17(IgG4) as disclosed herein) for use in any of the methods disclosed herein may be administered to a patient in need of the treatment once every week, e.g., via intravenous infusion. Alternatively, the anti-Galectin-9 antibody may be administered to the patient once every two weeks, e.g., via intravenous infusion. In some embodiments, the anti-Galectin-9 antibody is administered once every week for one cycle, once every week for two cycles, once every week for 3 cycles, once every week for 4 cycles, or once every week for more than 4 cycles. In other embodiments, the anti-Galectin-9 antibody is administered once every 2 weeks for one cycle, once every 2 weeks for two cycles, once every 2 weeks for 3 cycles, once every 2 weeks for 4 cycles, or once every 2 weeks for more than 4 cycles.

In some embodiments, the duration of treatment is 12-24 months or longer. In some embodiments, the cycles extend for a duration of 3 months to 6 months, or 6 months to 12 months or 12 months to 24 months or longer. In some embodiments, the cycle length is modified, e.g., temporarily or permanently to a longer duration, e.g., 3 weeks or 4 weeks.

Given that pro-tumor action of Galectin-9 is mediated through interaction with immune cells (e.g., interactions with lymphoid cells via TIM-3, CD44, and 41BB, and with macrophages via dectin-1 and CD206) and given that Galectin-9 is expressed in a large number of tumors, targeting Galectin-9, e.g., using a Galectin-9 binding antibody to inhibit interaction with its receptors provides a therapeutic approach that can be applied across a variety of different tumor types.

(ii) Combined Treatment with Anti-PD-1 Antibodies

Any of the methods disclosed herein may further comprise administering to the patient an effective amount of an anti-PD-1 antibody, for example, tislelizumab. Examples of PD-1 inhibitors include anti-PD-1 antibodies, such as pembrolizumab, nivolumab, tislelizumab, dostarlimab, and cemiplimab. Such checkpoint inhibitors can be administered simultaneously or sequentially (in any order) with the anti-Galectin-9 antibody according to the present disclosure. In some embodiments, the checkpoint molecule is PD-L1. Examples of PD-L1 inhibitors include anti-PD-L1 antibodies, such as durvalumab, avelumab, and atezolizumab.

In some embodiments, the antibody that binds PD-1 is tislelizumab. In some embodiments, the methods described herein comprise administration of tislelizumab to the subject at a dose of about 200 mg intravenously once every 3 weeks. In some embodiments, the methods described herein comprise administration of tislelizumab to the subject at a dose of about 400 mg intravenously once every 6 weeks. In some embodiments, the methods described herein comprise administration of tislelizumab to the subject at a dose of about 300 mg every 4 weeks. In some embodiments, tislelizumab is administered at about 300 mg intravenously every 4 weeks, in a 28-day cycle. Alternatively or in addition, tislelizumab is administered as an intravenous infusion, e.g., over approximately 30 minutes. In some embodiments, the antibody that binds PD-1 is dostarlimab. In some embodiments, the methods described herein comprise administration of dostarlimab to the subject at a dose of about 500 mg intravenously every three weeks or about 1000 mg intravenously every six weeks.

In some instances, the checkpoint inhibitor such as any of the anti-PD-1 antibodies (e.g., tislelizumab) disclosed herein and any of the anti-Galectin 9 antibodies disclosed herein such as G9.2-17(IgG4) may have same day administration. In some examples, the checkpoint inhibitor can be administered to a subject prior to administration of the anti-Galectin 9 antibody. In other instances, the administration of the checkpoint inhibitor, e.g., anti-PD-1 antibody, and the administration of the anti-Galectin 9 antibody are performed on two consecutive days. The checkpoint inhibitor, e.g., anti-PD-1 antibody, may be administered to the subject on the first day of dosing and the anti-Galectin-9 antibody can be administered to the subject on the subsequent day.

In other instances, the checkpoint inhibitor such as any of the anti-PD-1 antibodies disclosed herein may be administered about 1-7 days (e.g., 1 day, 2 day, 3 day, 4 day, 5 day, 6 day, or 7 day) prior to administration of the anti-Galectin 9 antibodies disclosed herein such as G9.2-17.

In some examples, the anti-Galectin 9 antibody can be administered to a subject prior to administration of the checkpoint inhibitor, e.g., an anti-PD-1 antibody. In other instances, the administration of the anti-Galectin 9 antibody and the administration of the checkpoint inhibitor, e.g., anti-PD-1 antibody, are performed on two consecutive days. The anti-Galectin-9 antibody may be administered to the subject on the first day of dosing and checkpoint inhibitor, e.g., anti-PD-1 antibody, can be administered to the subject on the subsequent day.

In other instances, the anti-Galectin-9 antibodies disclosed herein, such as G9.2-17, may be administered about 1-7 days (e.g., 1 day, 2 day, 3 day, 4 day, 5 day, 6 day, or 7 day) prior to administration of the checkpoint inhibitor, such as any of the anti-PD-1 antibodies disclosed herein.

In any of the method embodiments described herein, the anti-galectin-9 antibody can be administered (alone or in combination with an anti-PD-1 antibody such as tislelizumab) once every 2 weeks for one cycle, once every 2 weeks for two cycles, once every 2 weeks for three cycles, once every 2 weeks for four cycles, or once every 2 weeks for more than four cycles. In some embodiments, the treatment is 1 to 3 months, 3 to 6 months, 6 to 12 months, 12 to 24 months, or longer. In some embodiments, the treatment is once every 2 weeks for 1 to 3 months, once every 2 weeks for 3 to 6 months, once every 2 weeks for 6 to 12 months, or once every 2 weeks for 12 to 24 months, or longer.

In some examples, the method provided herein comprises administering to a subject in need of the treatment (e.g., a human patient having head and neck cancer, urothelial carcinoma, or other solid tumors as disclosed herein) an anti-Galectin-9 antibody such as G9.2-17(IgG4) at a dose of 2 mg/kg to 20 mg/kg once every week and an anti-PD-1 antibody such as tislelizumab at a dose of, e.g., 300 mg once every 4 weeks. In one example, the patient is given G9.2-17(IgG4) at a dose of 4 mg/kg once every week and tislelizumab at a dose of 300 mg once every 4 weeks. In another example, the patient is given G9.2-17(IgG4) at a dose of 6.3 mg/kg once every week and tislelizumab at a dose of 300 mg once every 4 weeks. In yet another example, the patient is given G9.2-17(IgG4) at a dose of 10 mg/kg once every week and tislelizumab at a dose of 300 mg once every 4 weeks. Alternatively, the patient is given G9.2-17(IgG4) at a dose of 16 mg/kg once every week and tislelizumab at a dose of 300 mg once every 4 weeks.

(iii) Patients for Treatment

A subject having any of the above noted cancers can be identified by routine medical examination, e.g., laboratory tests, organ functional tests, genetic tests, interventional procedure (biopsy, surgery) any and all relevant imaging modalities. In some embodiments, the subject to be treated by the method described herein is a human cancer patient who has undergone or is subjected to an anti-cancer therapy regimen delivered systemically and/or locally, for example, chemotherapy, radiotherapy, tumor-treating fields (TTFields), immunotherapy, biological therapy, small molecule inhibitors, anti-hormonal therapy, cell-based therapy, and/or surgery, in any combination or sequence of the outlined therapeutic modalities. In some embodiments, subjects have received prior immune-modulatory or any other anti-tumor agents or treatment modalities listed above. Non-limiting examples of such immune-modulatory agents include, but are not limited to as anti-PD-1, anti-PD-L1, anti-CTLA-4, anti-TIGIT, anti-PVRIG, anti-LAG-3, anti-CD47, anti-CD40, anti-CSFR1, anti-CD73, anti-SIRP, anti-A2AR, anti-OX40, anti-CD137, etc. In some embodiments, the subject shows disease progression through the treatment. In other embodiments, the subject is resistant to the treatment (either de novo or acquired). In some embodiments, such a subject is demonstrated as having advanced malignancies (e.g., inoperable or metastatic). Alternatively or in addition, in some embodiments, the subject has no standard therapeutic options available or ineligible for standard treatment options, which refer to therapies commonly used in clinical settings for treating the corresponding solid tumor.

Tumor-treating fields (TTFields) are a cancer treatment modality that uses alternating electric fields of intermediate frequency (˜100-500 kHz) and low intensity (1-3 V/cm) to disrupt cell division. In any of the embodiments described herein, the anti-Galectin-9 antibody, alone or in combination with a checkpoint inhibitor, such as an anti-PD-1 antibody, may be administered prior to, concurrent with, or after a tumor-treating fields (TTFields) regimen.

In some instances, the subject may be a human patient having a refractory disease, for example, a refractory head and neck cancer, or a refractory urothelial carcinoma. As used herein, “refractory” refers to the tumor that does not respond to or becomes resistant to a treatment. In some instances, the subject may be a human patient having a relapsed disease, for example, a relapsed head and neck cancer, or a relapsed urothelial carcinoma. As used herein, “relapsed” or “relapses” refers to the tumor that returns or progresses following a period of improvement (e.g., a partial or complete response) with treatment.

In some embodiments, the human patient to be treated by the methods disclosed herein meets one or more of the inclusion and exclusion criteria disclosed in Example 3 below. For example, the human patient may be 18 or older; having histologically confirmed unresectable metastatic or inoperable cancer (e.g., without standard therapeutic options), having a life expectancy >3 months, having recent archival tumor sample available for biomarker analysis (e.g., an archival species for Galectin-9 tumor tissue expression levels assessed by IHC); having a measurable disease, according to RECIST v1.1, having Eastern Cooperative Oncology Group (ECOG) performance status 0-1 or Karnofsky score >70; having no available standard of care options, having MSI-H (Microsatellite instability high and MSS (Microsatellite Stable); received at least one line of systemic therapy in the advanced/metastatic setting; having adequate hematologic and end organ function (defined in Example 1 below; e.g., e.g., neutrophil count ≥1×10⁹/L, platelet count ≥100×10⁹/L, for HCC in Part 1≥50×10⁹/L; hemoglobin ≥9.0 g/dL without transfusion in the previous week, Creatinine ≤1.5×ULN, AST (SGOT)≤3×ULN (≤5×ULN when HCC or hepatic metastases are present), ALT (SGPT)≤3×ULN (≤5×ULN when HCC or hepatic metastases present), Bilirubin ≤1.5×ULN (patients with known Gilbert's disease may have a bilirubin ≤3.0×ULN), Albumin ≥3.0 g/dL, INR and PTT≤1.5×ULN; and/or amylase and lipase ≤1.5×ULN)); having completed treatment for brain metastases if any (see Example 1 below); having no evidence of active infection and no serious infection within the past month; having at least four (4) weeks s or 5 half lives (whichever is shorter) since the last dose of anti-cancer therapy before the first anti-Gal-9 antibody administration.

Alternatively or in addition, the subject suitable for the treatment disclosed herein may not have one or more of the following: diagnosed with metastatic cancer of an unknown primary; any active uncontrolled bleeding, and any patients with a bleeding diathesis (e.g., active peptic ulcer disease); receiving any other investigational agents within 4 weeks or 5 half-lives of anti-galectin-9 antibody administration; receiving radiation therapy within 4 weeks of the first dose of the anti-Galectin-9 antibody, except for palliative radiotherapy to a limited field, such as for the treatment of bone pain or a focally painful tumor mass; having fungating tumor masses; having active clinically serious infection>grade 2 NCI-CTCAE version 5.0; having symptomatic or active brain metastases; having ≥CTCAE grade 3 toxicity (see details and exceptions in Example 1); having history of second malignancy (see exceptions in Example 1); having evidence of severe or uncontrolled systemic diseases, congestive cardiac failure; having serious non-healing wound, active ulcer or untreated bone fracture; having uncontrolled pleural effusion, pericardial effusion, or ascites requiring recurrent drainage procedures; having spinal cord compression not definitively treated with surgery and/or radiation. Leptomeningeal disease, active or previously treated; having significant vascular disease; having active auto-immune disorder (see exceptions in Example 1); require systemic immunosuppressive treatment; having tumor-related pain (>grade 3) unresponsive to broad analgesic interventions (oral and/or patches); having uncontrolled hypercalcemia, despite use of bisphosphonates; having any history of an immune-related Grade 4 adverse event attributed to prior checkpoint inhibitor therapy (CIT); received an organ transplant(s); and/or on undergoing dialysis; and/or having Child-Pugh score ≥7. In some instances, the human patient may not have metastatic hepatocellular carcinoma that progressed while receiving at least one previous line of systemic therapy; have refuse or not tolerated sorafenib; or have had standard therapy considered ineffective, intolerable, or inappropriate or for which no effective standard therapy is available.

Alternatively or in addition, the human patient subject to any treatment disclosed herein may be free of: (i) metastatic cancer of an unknown primary, (ii) clinically significant, active uncontrolled bleeding, any bleeding diathesis (e.g., active peptic ulcer disease); (iii) radiation therapy within 4 weeks of the first dose of the treatment, (iv) with fungating tumor masses; (v) ≥CTCAE grade 3 toxicity (except alopecia and vitiligo) due to prior cancer therapy; (v) history of second malignancy, (vi) evidence of severe or uncontrolled systemic diseases, congestive cardiac failure>New York Heart Association (NYHA) class 2, or myocardial infarction (MI) within 6 months, (vii) serious non-healing wound, active ulcer, or untreated bone fracture; (viii) uncontrolled pleural effusion, pericardial effusion, or ascites requiring recurrent drainage procedures; (ix) history of severe allergic, anaphylactic, or other hypersensitivity reactions to chimeric or humanized antibodies or fusion proteins; (x) significant vascular disease (e.g., aortic aneurysm requiring surgical repair or recent arterial thrombosis) within 6 months of the treatment, history of pulmonary embolism, stroke or transient ischemic attack within 3 months prior to the treatment, and/or history of abdominal fistula or gastrointestinal perforation within 6 months prior to the treatment; (xi) active auto-immune disorder (except type I diabetes, hypothyroidism requiring only hormone replacement, vitiligo, psoriasis, or alopecia); (xii) requires systemic immunosuppressive treatment; (xii) tumor-related pain (>grade 3) unresponsive to broad analgesic interventions (oral and/or patches); (xiii) uncontrolled hypercalcemia, despite use of bisphosphonates; (xiv) received organ transplant(s).

In some instances, the subject is a human patient having an elevated level of Galectin-9 as relative to a control level. The level of Galectin-9 can be a plasma or serum level of Galectin-9 in the human patient. In other examples, the level of Galectin-9 is the level of Galectin-9 of cancer cells within the tumor. In other examples, the level of Galectin-9 is the level of Galectin-9 of immune cells within the tumor. In other examples, the level of Galectin-9 can be the level of cell-surface Galectin-9, for example the level of Galectin-9 on cancer cells. In one example, the level of Galectin-9 can be the level of Galectin-9 expressed cancer cells, e.g., on the surface of cancer cells, or Galectin-9 expressed in immune cells, measured in patient-derived organotypic tumor spheroids (PDOT), which can be prepared by, e.g., the method disclosed in Examples below. A control level may refer to the level of Galectin-9 in a matched sample of a subject of the same species (e.g., human) who is free of the solid tumor. In some examples, the control level represents the level of Galectin-9 in healthy subjects. In some embodiments, the control level may be a baseline level prior to treatment.

To identify such a subject, a suitable biological sample can be obtained from a subject who is suspected of having the solid tumor and the biological sample can be analyzed to determine the level of Galectin-9 contained therein (e.g., free, cell-surface expressed, or total) using conventional methods, e.g., ELISA or FACS. In some embodiments, organoid cultures are prepared, e.g., as described herein, and used to assess Galectin-9 levels in a subject. Single cells derived from certain fractions obtained as part of the organoid preparation process are also suitable for assessment of Galectin-9 levels in a subject. In some instances, an assay for measuring the level of Galectin-9, either in free form or expressed on cell surface, involves the use of an antibody that specifically binds the Galectin-9 (e.g., specifically binds human Galectin-9). Any of the anti-Galectin-9 antibodies known in the art can be tested for suitability in any of the assays described above and then used in such assays in a routine manner. In some embodiments, an antibody described herein (e.g., a G9.2-17 antibody) can be used in such as assay. In some embodiments, an antibody described in U.S. Pat. No. 10,344,091 and WO2019/084553, the relevant disclosures of each of which are incorporated by reference for the purpose and subject matter referenced herein. In some examples, the anti-Galectin-9 antibody is a Fab molecule. Assay methods for determining Galectin-9 levels as disclosed herein are also within the scope of the present disclosure.

### (iv) Responses to Treatment

Efficacy of the treatment as disclosed herein can be assessed via routine practice. In some embodiments, any of the methods disclosed herein can increase anti-tumor activity (e.g., reduce cell proliferation, tumor growth, tumor volume, and/or tumor burden or load or reduce the number of metastatic lesions over time) by at least about 10%, 20%, 25%, 30%, 40%, 50%, 60%, 70%, 75%, 80%, 85%, 90%, 95%, or more as compared to levels prior to treatment or in a control subject. In some embodiments, reduction is measured by comparing cell proliferation, tumor growth, and/or tumor volume in a subject before and after administration of the pharmaceutical composition. In some embodiments, the method disclosed herein may improve one or more symptoms of the cancer by at least about 10%, 20%, 30%, 40%, 50%, 60%, 70%, 80%, 90%, 95%, or more. In some embodiments, before, during, and after the administration of the pharmaceutical composition, cancerous cells and/or biomarkers in a subject are measured in a biological sample, such as blood, serum, plasma, urine, peritoneal fluid, and/or a biopsy from a tissue or organ. In some embodiments, the methods include administration of the compositions of the invention to reduce tumor volume, size, load or burden in a subject to an undetectable size, or to less than about 1%, 2%, 5%, 10%, 20%, 25%, 30%, 40%, 50%, 60%, 70%, 75%, 80%, or 90% of the subject's tumor volume, size, load or burden prior to treatment. In other embodiments, methods are provided for reducing the cell proliferation rate or tumor growth rate in a subject to an undetectable rate, or to less than about 1%, 2%, 5%, 10%, 20%, 25%, 30%, 40%, 50%, 60%, 70%, 75%, 80%, or 90% of the rate prior to treatment. In other embodiments, methods include administration of the compositions of the invention to reduce the development of or the number or size of metastatic lesions in a subject to an undetectable rate, or to less than about 1%, 2%, 5%, 10%, 20%, 25%, 30%, 40%, 50%, 60%, 70%, 75%, 80%, or 90% of the rate prior to treatment.

A response to treatment, e.g., a treatment of a solid tumor as described herein, can be assessed according to RECIST or the RECIST 1.1 criteria and/or irRC, irRECIST, iRECIST, imRECISTPDAC, as described in Example 1 below and Eisenhower et al., New response evaluation criteria in solid tumours: Revised RECIST guideline (version 1.1); European Journal Of Cancer 45 (2009) 228-247; or Borcoman et al., Annals of Oncology 30: 385-396, 2019; Nishino et al., Clin Cancer Res 2013; 19(14): 3936-3943, the contents of each of which is herein incorporated by reference in its entirety.

In some embodiments, methods are provided for improving and or controlling the overall response/tumor burden/tumor size (e.g., at approximately 2, 3, 6 or 12 months, or a later time) comprising administering an anti-Galectin-9 antibody described herein, e.g., as compared to a baseline level obtained prior to initiation of G9.2-17 IgG4 treatment regimen. In some embodiments, the methods are for improving and or controlling the overall response/tumor burden/tumor size at approximately 2 months. In some embodiments, where the anti-Galectin-9 antibody (e.g., G9.2-17(IgG4)) is administered in a combination regimen with a checkpoint inhibitor, e.g., an anti-PD-1 antibody such as tislelizumab, can improve or control the overall response/tumor burden/tumor size (e.g., at approximately 2, 3, 6 or 12 months, or a later time), e.g., as compared to a baseline level obtained prior to initiation of treatment. In some embodiments, methods are provided, which result in a complete response, a partial response or stable disease (e.g., as measured at approximately 2 months, 3 months, 6 months or 12 months, or at a later time or at any other clinically indicated time point), comprising administering an anti-Galectin-9 antibody described herein. Such a response can be temporary over a certain time period or permanent.

In some embodiments, a method as disclosed herein may improve the likelihood of a complete response, a partial response or stable disease (e.g., as measured at approximately 2 months, 3 months, 6 months or 12 months, or at a later time or at any other clinically indicated time point), e.g., as compared to a baseline level obtained prior to initiation of G9.2-17 IgG4 treatment regimen. Such a response can be temporary over a certain time period or permanent. In some embodiments, treating can result in reduced or attenuated progressive disease (e.g., as measured at approximately 2 months, 3 months, 6 months or 12 months, or at a later time or at any other clinically indicated time point), e.g., as compared to a baseline level obtained prior to initiation of G9.2-17 IgG4 treatment regimen. Such an attenuation may be temporary or permanent. In any of these embodiments, anti-Galectin-9 antibody may be administered in combination with a checkpoint inhibitor, e.g., an anti-PD-1 antibody.

In some embodiments, a method as disclosed herein may attenuate disease progression or reducing progressive disease (e.g., as measured at approximately 3 months, 6 months or 12 months, or at a later time or at any other clinically indicated time point). The method comprising administering to the subject a therapeutically effective amount of an anti-Galectin-9 antibody as disclosed herein. In any of these embodiments, the anti-Galectin-9 antibody may be administered in combination with a checkpoint inhibitor, e.g., an anti-PD-1 antibody.

In any of the methods described herein, partial response, stable disease, complete response, a partial response, stable disease, progressive disease, disease progressing (e.g., as measured at approximately 2 months, 3 months, 6 months or 12 months, or at a later time or at any other clinically indicated time point), can be assessed according to irC criteria, RECIST criteria, RECIST1.1., irRECIST or iRECIST, or imRECIST criteria, or other criteria known in the art (see, e.g., Borcoman et al., Annals of Oncology 30: 385-396, 2019′ iRC: Hoos et al., J. Immunother. 30 (1): 1-15).

A partial response is a decrease in the size of a tumor, or in the extent of cancer in the body, i.e., the tumor burden, in response to treatment as compared to a baseline level before the initiation of the treatment. For example, according to the RECIST response criteria, a partial response is defined as at least a 30% decrease in the sum of diameters of target lesions, taking as reference the baseline sum diameters. Progressive disease is a disease that is growing, spreading, or getting worse. For example, according to the RECIST response criteria, progressive disease includes disease in which at least a 20% increase in the sum of diameters of target lesions is observed, and the sum must also demonstrate an absolute increase of at least 5 mm. Additionally, the appearance of one or more new lesions is also considered progression. A tumor that is neither decreasing nor increasing in extent or severity as compared to a baseline level before initiation of the treatment is considered stable disease. For example, according to the RECIST response criteria, stable disease occurs when there is neither sufficient shrinkage to qualify for partial response nor sufficient increase to qualify for progressive disease, taking as reference the smallest sum diameters while on study.

In some embodiments, the disclosure provides methods for reducing or maintaining tumor size in a subject, including a human subject, (e.g., as measured at approximately 2 months, 3 months, 6 months or 12 months, or at a later time or at any other clinically indicated time point) either permanently or over a minimum time period, relative to a baseline tumor size prior to initiation of the treatment in the subject, the method comprising administering to the subject a therapeutically effective amount of an anti-Galectin-9 antibody alone or in combination with a checkpoint inhibitor, e.g., an anti-PD-1 antibody. Tumor size, e.g., the diameters of tumors, can be measured according to methods known in the art, which include measurements from CT and MRI images in combination with various software tools, according to specific measurement protocols, e.g., as described in Eisenhower et al., referenced above. Accordingly, in some embodiments, tumor size is measured in regularly scheduled restaging scans (e.g., CT with/without contrast, MRI with/without contrast, PET-CT (diagnostic CT) and/or X-ray, ultrasound and/or other relevant imaging modality). In some embodiments, tumor size reduction, maintenance of tumor size refers to the size of target lesions. In some embodiments, tumor size reduction, maintenance of tumor size refers to the size of non-target lesions. According to RECIST 1.1, when more than one measurable lesion is present at baseline, all lesions up to a maximum of five lesions total (and a maximum of two lesions per organ) representative of all involved organs should be identified as target lesions. All other lesions (or sites of disease) including pathological lymph nodes should be identified as non-target lesions.

In some embodiments, the disclosure provides methods for increasing the likelihood of reducing or maintaining a tumor burden (e.g., as measured at approximately 2 months, 3 months, 6 months or 12 months, or at a later time or at any other clinically indicated time point), the methods comprising administering to the subject a therapeutically effective amount of an anti-Galectin-9 antibody as disclosed herein, alone or in combination with a checkpoint inhibitor, e.g., an anti-PD-1 antibody such as tislelizumab. In some embodiments, treating can result in in a greater likelihood of a reduction of tumor burden, or maintenance of tumor burden, (e.g., as measured at approximately 2 months, 3 months, 6 months or 12 months, or at a later time or at any other clinically indicated time point). As used herein, tumor burden refers to amount of cancer, the size or the volume of the tumor in the body of a subject, accounting for all sites of disease. Tumor burden can be measured using methods known in the art, including but not limited to, FDG positron emission tomography (FDG-PET), magnetic resonance imaging (MRI), and optical imaging, comprising bioluminescence imaging (BLI) and fluorescence imaging (FLI).

In some embodiments, the methods described herein increase in the time to disease progression or in progression free survival (e.g., as measured at approximately 2 months, 3 months, 6 months or 12 months, or at a later time or at any other clinically indicated time point post initiation of treatment). Progression free survival can be either permanent or progression free survival over a certain amount of time. In some embodiments, the methods provide a greater likelihood of progression free survival (either permanent progression free survival or progression free survival over a certain amount of time, e.g., 3, 6 or 12 months or e.g., as measured at approximately 2 months, 3 months, 6 months or 12 months, or at a later time or at any other clinically indicated time point post initiation of treatment). Progression-free survival (PFS) is defined as the time from random assignment in a clinical trial, e.g., from initiation of a treatment to disease progression or death from any cause. In some embodiments, the methods achieve longer survival or greater likelihood of survival, e.g., at a certain time, e.g., at 6 or 12 months.

A response to treatment, e.g., a treatment of a solid tumor as described herein, can be assessed according to iRECIST criteria, as described in Seymour et al, iRECIST: guidelines for response criteria for use in trials; The Lancet, Vol 18, March 2017, the contents of which is herein incorporated by reference in its entirety. iRECIST was developed for the use of modified RECIST1.1 criteria specifically in cancer immunotherapy trials, to ensure consistent design and data collection and can be used as guidelines to a standard approach to solid tumor measurements and definitions for objective change in tumor size for use in trials in which an immunotherapy is used. iRECIST is based on RECIST 1.1. Responses assigned using iRECIST have a prefix of “i” (ie, immune)—e.g., “immune” complete response (iCR) or partial response (iPR), and unconfirmed progressive disease (iUPD) or confirmed progressive disease (iCPD) or stable disease (iSD) to differentiate them from responses assigned using RECIST 1.1, and all of which are defined in Seymour et al. RECIST 1.1. In some embodiments criteria can be compared to baseline levels prior to initiation of treatment. In any of these embodiments, the anti-Galectin-9 antibody may be administered alone or in combination with a checkpoint inhibitor, e.g., an anti-PD-1 antibody such as those disclosed herein.

Accordingly, in some embodiments, the disclosure provides methods for improving overall response (iOR) or achieving “immune” complete response (iCR), a partial response (iPR) or stable disease (iSD) (e.g., as measured at approximately 2 months, 3 months, 6 months or 12 months, or at a later time or at any other clinically indicated time point), as compared to the baseline level of disease prior to initiation of the treatment. The reduction in the “immune” response, e.g., iCR, iPR, or iSD can be temporary over a certain time period or permanent. In some embodiments, treating can improve the likelihood of a complete response (iCR), a partial response (iPR) or stable disease (iSD) (e.g., as measured at approximately 2 months, 3 months, 6 months or 12 months, or at a later time or at any other clinically indicated time point), e.g., In some embodiments, the disclosure provides methods for attenuating disease progression or reducing progressive disease, e.g., reducing unconfirmed progressive disease (iUPD) or reducing confirmed progressive disease (iCPD)) (e.g., as measured at approximately 2 months, 3 months, 6 months or 12 months, or at a later time or at any other clinically indicated time point), the method comprising administering to the subject a therapeutically effective amount of an anti-Galectin-9 antibody as disclosed herein. Any of these above mentioned iRECIST criteria can be compared to baseline levels prior to initiation of treatment. In any of these methods the anti-Galectin-9 antibody may be administered alone or in combination with a checkpoint inhibitor, e.g., an anti-PD-1 antibody.

The reduction in iUPD or iCPD can be temporary over a certain time period or permanent. In some embodiments, treating can result in greater likelihood of overall reduction in unconfirmed progressive disease (iUPD) or confirmed progressive disease (iCPD) (e.g., as measured at approximately 2 months, 3 months, 6 months or 12 months, or at a later time or at any other clinically indicated time point In some embodiments, the disclosure provides methods for reducing the number of new lesions in a subject, including a human subject, according to iRECIST criteria (e.g., as measured at approximately 2 months, 3 months, 6 months or 12 months, or at a later time or at any other clinically indicated time point), the methods comprising administering to the subject a therapeutically effective amount of an anti-Galectin-9 antibody as disclosed herein. Reduced number of lesions can be relative to baseline levels prior to initiation of treatment, and the reduction can be temporary over a certain time period or permanent. In any of these embodiments, the anti-Galectin-9 antibody may be administered in combination with a checkpoint inhibitor, e.g., an anti-PD-1 antibody.

Additional criteria can be used to measure a treatment response. For example, tumor burden can be measured according to the irRC criteria (Hoos et al., 2007). In the irRC, tumor burden is measured by combining ‘index’ lesions with new lesions, i.e., new lesions are considered a change in tumor burden. In the irRC, an immune-related Complete Response (irCR) is the disappearance of all lesions, measured or unmeasured, and no new lesions; an immune-related Partial Response (irPR) is a 50% drop in tumor burden from baseline as defined by the irRC; and immune-related Progressive Disease (irPD) is a 25% increase in tumor burden from the lowest level recorded. Everything else is considered immune-related Stable Disease (irSD).

Immune-related RECIST (irRECIST) is based on unidimensional measurements of RECIST, and Specific immune-related criteria were further redefined in the irRECIST. Recently, new criteria were evaluated based on atezolizumab data in NSCLC, the immune-modified RECIST (imRECIST), requiring a confirmation of disease progression at least 4 weeks after initial assessment (Hodi et al, JCO 2018; 36(9): 850-858). For a comparison of RECIST 1.1., irRC, irRECIST, iRECIST and imRECIST, see, e.g., FIG. 4 in Borcoman et al., Annals of Oncology 30: 385-396, 2019; Nishino et al., Clin Cancer Res 2013; 19(14): 3936-3943, the contents of which is herein incorporated by reference in its entirety. Any of these criteria are suitable in determining response rate in any of the methods described herein.

### (v) Monitoring Adverse Events and Modifying Treatment Conditions

In addition, a subject being treated by any of the anti-galectin-9 antibodies disclosed herein (e.g., G9.2-17), either alone or in combination with a checkpoint inhibitor (e.g., an anti-PD-1 such as tislelizumab) as disclosed herein may be monitored for occurrence of adverse effects (for example, severe adverse effects). Exemplary adverse effects to monitor are provided in Example 3 below. If occurrence of adverse effects is observed, treatment conditions may be changed for that subject. For example, the dose of the anti-galectin-9 antibody may be reduced and/or the dosing interval may be extended. Suitability and extent of reduction may be assessed by a qualified clinician. In one embodiment, a reduction level of 30 or 50% of the previous dose level is implemented. In one specific example, a reduction level as per clinician's assessment or at least by 30% is implemented (to dose level 1, the level at first dose reduction). If required, one more dose reduction by 30% of dose level −1 is implemented (dose level −2, the level at second dose reduction). In another example, one more dose reduction by 50% of dose level −1 is implemented (dose level −2). In some embodiments, one or more dose reductions by about 10% to about 80% of a previous dose level are implemented. In some embodiments, one or more dose reductions by about 10% to about 20%, about 20% to about 30%, about 30% to about 40%, about 40% to about 50%, about 50% to about 60%, or about 70% to about 80% of a previous dose level are implemented. In some embodiments, one or more dose reductions by 10% to 20%, 20% to 30%, 30% to 40%, 40% to 50%, 50% to 60%, or 70% to 80% of a previous dose level are implemented. In some embodiments, one or more dose reductions by about 10%, by about 20%, by about 30%, by about 40%, by about 50%, by about 60%, by about 70%, or by about 80% of a previous dose level are implemented. In some embodiments, one or more dose reductions by 10%, by 20%, by 30%, by 40%, by 50%, by 60%, by 70%, or by 80% of a previous dose level are implemented. Alternatively or in addition, the dose of the checkpoint inhibitor can be reduced and/or the dosing interval of the checkpoint inhibitor may be extended.

In some instances (e.g., occurring of life-threatening adverse effects), the treatment may be terminated.

In some instances, the dose of the anti-Galectin-9 antibody such as G9.2-17(IgG4) and/or the dose of the anti-PD-1 antibody such as tislelizumab may be reduced if an adverse effect is observed in a patient. In some instances, the dose may be reduced by 50%. When needed, the dose may further reduce by 50%. See, e.g., Example 3 below.

### (vi) Biomarkers for Assessing Response to Treatment

Response to treatment can also be characterized by one or more of immunophenotype in blood and tumors, cytokine profile (serum), soluble galectin-9 levels in blood (serum or plasma), galectin-9 tumor tissue expression levels and pattern of expression by immunohistochemistry (tumor, stroma, immune cells), tumor mutational burden (TMB), PD-L1 expression (e.g., by immunohistochemistry), mismatch repair status, or tumor markers relevant for the disease (e.g., as measured at approximately 3 months, 6 months or 12 months, or at a later time or at any other clinically indicated time point). Examples of such tumor markers include, but are not limited to, CA15-3, CA-125, CEA, CA19-9, alpha fetoprotein. These parameters can be compared to baseline levels prior to initiation of treatment. In any of these embodiments, the anti-Galectin-9 antibody may be administered alone or in combination with a checkpoint inhibitor, e.g., an anti-PD-1 antibody.

In any of the methods disclosed herein, the subject may examined for one or more of the following features before, during, and/or after the treatment: (a) one or more tumor markers in blood samples from the subject, optionally wherein the one or more tumor markers comprise CA15-3, CA-125, CEA, CA19-9, and/or alpha fetoprotein, and any other tumor-type specific tumor markers; (b) cytokine profile; and (c) galectin 9 serum/plasma levels, d) peripheral blood mononuclear cell immunophenotyping, e) tumor tissue biopsy/excisional specimen multiplex immunophenotyping, f) tumor tissue biopsy/excisional specimen galectin-9 expression levels and pattern, g) any other immune score test such as: PD-L1 immunohistochemistry, tumor mutational burden (TMB), tumor microsatellite instability status, as well as panels such as: Immunoscore®-HalioDx, ImmunoSeq-Adaptive Biotechnologies, TIS, developed on the NanoString nCounter® gene expression system, 18-gene signature, PanCancer IO 360™ assay (NanoString Technologies) etc. Other suitable biomarkers specific to the target tumor such as PDAC may also be used. In one non-limiting example, PD-L1 (SP263) (Roche, Ventana) can be used for detection of PD-L1 in cancer tissues using immunohistochemistry.

In some embodiments, the methods are described herein for changing levels of immune cells and immune cell markers in the blood or in tumors, e.g., immune activation, comprising an anti-Gal-9 antibody is administered alone or in combination with a checkpoint inhibitor, e.g., an anti-PD-1 antibody. Such changes can be measured in patient blood and tissue samples using methods known in the art, such as multiplex flow cytometry and multiplex immunohistochemistry. For example, a panel of phenotypic and functional PBMC immune markers can be assessed at baseline prior to commencement of the treatment and at various time point during treatment. Table 2 lists non-limiting examples of markers useful for these assessment methods. Flow cytometry (FC) is a fast and highly informative method of choice technology to analyze cellular phenotype and function and has gained prominence in immune phenotype monitoring. It allows for the characterization of many subsets of cells, including rare subsets, in a complex mixture such as blood, and represents a rapid method to obtain large amounts of data. Advantages of FC are high speed, sensitivity, and specificity. Standardized antibody panels and procedures can be used to analyze and classify immune cell subtypes. Multiplex IHC is a powerful investigative tool, which provides objective quantitative data describing the tumor immune context in both immune subset number and location and allows for multiple markers to be assessed on a single tissue section. Computer algorithms can be used to quantify IHC-based biomarker content from whole slide images of patient biopsies, combining chromogenic JHC methods and stains with digital pathology approaches.

Accordingly, in some embodiments, methods are described herein, for modulating an immune response, e.g., modulation of immune activation markers such as those in Table 2 comprising administering an anti-gal9 antibody alone or in combination with a checkpoint inhibitor therapy. In some embodiments, modulation comprises in one or more of (1) an increase in more CDT cells in plasma or tumor tissue, (2) a reduction in T regulatory cells (Tregs) in plasma or tumor tissue, (3) an increase in M1 macrophages in plasma or tumor tissue and (4) a decrease in MDSCs in plasma or tumor tissue, and (5) a decrease in M2 macrophages in plasma or tumor tissue (e.g., as measured at approximately 2 months, 3 months, 6 months or 12 months, or at a later time or at any other clinically indicated time point). In some embodiments, the markers that are assessed using the techniques described above or known in the art are selected from CD4, CD8 CD14, CD11b/c, and CD25. These parameters can be compared to baseline levels prior to initiation of treatment.

TABLE 2
PBMC phenotyping markers

| PBMC phenotyping markers | | PBMC phenotyping markers | |
|---|---|---|---|
| CD3 | Total T cells | CD16 | NK cells |
| CD4 | CD4+ T cells | CD11b | Monocytes/macrophages |
| CD8 | CD8+ T cells | CD11c | Monocytes/macrophages, DCs |
| CD25 | Treg activation | CD14 | Monocyte subsets, macrophages |
| CD27 | T cell maturation; B cell | CD33 | Total myeloid cells |
| | naïve/memory | | |
| CD38 | T cell maturation; B cell | FceR1 a | Antigen presenting DC cells |
| | naïve/memory | | |
| CD45RA | Naïve/memory cells | CD19 | Total B cells |
| CD45RO | Naïve/memory cells | T-bet | T cells subsets |
| CD56 | NKT/NK cells (T cell subset) | gdTCR | Gamma delta T cells |
| CD127 | T cell subsets | CD274 (PDL-1) | Checkpoint |
| CD152 (CLTA-4) | Checkpoint | Tim-3 | Checkpoint |
| CD279 (PD-1) | Checkpoint | TCRVa24-Ja18 | INKT cells |
| FoxP3 | Treg cells | Live/dead | General |
| HLA-DR | Activation/Antigen presentation | CD45 | General |

(vii) Modulating Immune Responses

In some embodiments, methods are described herein, comprising administering an anti-gal9 alone or in combination with a checkpoint inhibitor therapy, for modulating proinflammatory and anti-inflammatory cytokines. In some embodiments, methods are provided for one or more of (1) increasing levels of IFNgamma in plasma or tumor tissue; (2) increasing levels of TNFalpha in plasma or tumor tissue; (3) decreasing levels of IL-10 in plasma or tumor tissue (e.g., as measured at approximately 3 months, 6 months or 12 months, or at a later time or at any other clinically indicated time point). These parameters can be compared to baseline levels prior to initiation of treatment.

In some embodiments, cytokine levels or immune cell levels may be assessed between a pre dose 1 tumor biopsy and repeat biopsy conducted at a feasible time. In some embodiments, cytokine levels or immune cell levels may be assessed between 2 repeat biopsies. In some embodiments, methods are provided for modulating one or more of soluble galectin-9 levels in blood (serum or plasma), or galectin-9 tumor tissue expression levels and pattern of expression by immunohistochemistry (tumor, stroma, immune cells), (e.g., as measured at approximately 3 months, 6 months or 12 months, or at a later time or at any other clinically indicated time point). In some embodiments, the methods decrease soluble galectin-9 levels in blood (serum or plasma), or galectin-9 tumor tissue expression levels or pattern of expression by immunohistochemistry (tumor, stroma, immune cells) (e.g., as measured at approximately 3 months, 6 months or 12 months, or at a later time or at any other clinically indicated time point). Galectin-9 levels can be compared to baseline levels prior to initiation of treatment. In some embodiments, Galectin-9 levels may be compared to a control group not receiving the treatment or healthy subjects. In any of these embodiments, the anti-Galectin-9 antibody may be administered alone or in combination with a checkpoint inhibitor, e.g., an anti-PD-1 antibody. In some embodiments, methods for modulating PD-L1 expression are provided, e.g., as assessed by immunohistochemistry, comprising administering an anti-Galectin-9 antibody, alone or in combination with a checkpoint inhibitor, e.g., an ant-Galectin-9 antibody. In some embodiments, the methods modulate in one or more tumor markers (increase or decrease) relevant for the disease (e.g., as measured at approximately 2 months, 3 months, 6 months or 12 months, or at a later time or at any other clinically indicated time point). Examples of such tumor markers include, but are not limited to, CA15-3, CA-125, CEA, CA19-9, alpha fetoprotein. These parameters can be compared to baseline levels prior to initiation of treatment. In any of these embodiments, the anti-Galectin-9 antibody may be administered alone or in combination with a checkpoint inhibitor, e.g., an anti-PD-1 antibody.

In some embodiments, the disclosure provides methods of modulating an immune response in a subject. As used herein, the term “immune response” includes T cell-mediated and/or B cell-mediated immune responses that are influenced by modulation of immune cell activity, for example, T cell activation. In one embodiment of the disclosure, an immune response is T cell mediated. As used herein, the term “modulating” means changing or altering, and embraces both upmodulating and downmodulating. For example, “modulating an immune response” means changing or altering the status of one or more immune response parameter(s). Exemplary parameters of a T cell mediated immune response include levels of T cells (e.g., an increase or decrease in effector T cells) and levels of T cell activation (e.g., an increase or decrease in the production of certain cytokines). Exemplary parameters of a B cell mediated immune response include an increase in levels of B cells, B cell activation and B cell mediated antibody production.

When an immune response is modulated, some immune response parameters may decrease and others may increase. For example, in some instances, modulating the immune response causes an increase (or upregulation) in one or more immune response parameters and a decrease (or downregulation) in one or more other immune response parameters, and the result is an overall increase in the immune response, e.g., an overall increase in an inflammatory immune response. In another example, modulating the immune response causes an increase (or upregulation) in one or more immune response parameters and a decrease (or downregulation) in one or more other immune response parameters, and the result is an overall decrease in the immune response, e.g., an overall decrease in an inflammatory response. In some embodiments an increase in an overall immune response, i.e., an increase in an overall inflammatory immune response, is determined by a reduction in tumor weight, tumor size or tumor burden or any RECIST or iRECIST criteria described herein. In some embodiments an increase in an overall immune response is determined by increased level(s) of one or more proinflammatory cytokine(s), e.g., including two or more, three or more, etc. or a majority of proinflammatory cytokines (one or more, two or more, etc. or a majority of anti-inflammatory and/or immune suppressive cytokines and/or one or more of the most potent anti-inflammatory or immune suppressive cytokines either decrease or remain constant). In some embodiments an increase in an overall immune response is determined by increased levels of one or more of the most potent proinflammatory cytokines (one or more anti-inflammatory and/or immune suppressive cytokines including one or more of the most potent cytokines either decrease or remain constant). In some embodiments an increase in an overall immune response is determined by decreased levels of one or more, including a majority of, immune suppressive and/or anti-inflammatory cytokines (the levels of one or more, or a majority of, proinflammatory cytokines, including e.g., the most potent proinflammatory cytokines, either increase or remain constant). In some embodiments, an increase in an overall immune response is determined by increased levels of one or more of the most potent anti-inflammatory and/or immune suppressive cytokines (one or more, or a majority of, proinflammatory cytokines, including, e.g., the most potent proinflammatory cytokines either increase or remain constant). In some embodiments an increase in an overall immune response is determined by a combination of any of the above. Also, an increase (or upregulation) of one type of immune response parameter can lead to a corresponding decrease (or downregulation) in another type of immune response parameter. For example, an increase in the production of certain proinflammatory cytokines can lead to the downregulation of certain anti-inflammatory and/or immune suppressive cytokines and vice versa.

In some embodiments, the disclosure provides methods for modulating an immune response (e.g., as measured at approximately 2 months, 3 months, 6 months or 12 months, or at a later time or at any other clinically indicated time point) in a subject, including a human subject, comprising administering to the subject a therapeutically effective amount of an anti-Galectin-9 antibody as disclosed herein. In some embodiments, the disclosure provides methods for modulating levels of immune cells and immune cell markers, including but not limited to those described herein in Table 2, e.g., as compared to baseline levels prior to initiation of treatment, e.g., as compared to a baseline level obtained prior to initiation of the anti-Gal9 antibody treatment regimen, in the blood or in tumors of a subject, including a human subject, comprising administering to the subject a therapeutically effective amount of an anti-Galectin-9 antibody as disclosed herein. In some embodiments, the overall result of modulation is upregulation of proinflammatory immune cells and/or down regulation of immune-suppressive immune cells. In some embodiments, the disclosure provides methods for modulating levels of immune cells, wherein the modulating encompasses one or more of (1) increasing CD8 cells in plasma or tumor tissue, (2) reducing Tregs in plasma or tumor tissue, (3) increasing M1 macrophages in plasma or tumor tissue and (4) decreasing MDSC in plasma or tumor tissue, and (5) decreasing in M2 macrophages in plasma or tumor tissue, and wherein the methods comprise administering to the subject a therapeutically effective amount of an anti-Galectin-9 antibody as disclosed herein. In some embodiments, the markers to assess levels of such immune cells include but are not limited to CD4, CD8 CD14, CD11b/c, and CD25. In some embodiments, the disclosure provides methods for modulating levels of proinflammatory and immune suppressive cytokines (e.g., as measured at approximately 2 months, 3 months, 6 months or 12 months, or at a later time or at any other clinically indicated time point), e.g., as compared to baseline levels prior to initiation of treatment, in the blood or in tumors of a subject, including a human subject, comprising administering to the subject a therapeutically effective amount of an anti-Galectin-9 antibody as disclosed herein. In some embodiments, the overall result of modulation is upregulation of proinflammatory cytokines and/or down regulation of immune-suppressive cytokines. In some embodiments, the disclosure provides methods for modulating levels of cytokines cells, wherein the modulating encompasses one or more of (1) increasing levels of IFNgamma in plasma or tumor tissue; (2) increasing levels of TNFalpha in plasma or tumor tissue; (3) decreasing levels of IL-10 in plasma or tumor tissue.

In some embodiments, the disclosure provides methods for changing one or more of soluble galectin-9 levels in blood (serum or plasma), or in galectin-9 tumor tissue expression levels and pattern of expression by immunohistochemistry (tumor, stroma, immune cells) (e.g., as measured at 2 weeks, 4 weeks, 1 month, 2 month, 3 months, 6 months or 12 months, or at a later time or at any other clinically indicated time point), comprising administering to the subject a therapeutically effective amount of an anti-Galectin-9 antibody as disclosed herein. In some embodiments of the methods, one or more of soluble galectin-9 levels in blood (serum or plasma), or in galectin-9 tumor tissue expression levels and pattern of expression by immunohistochemistry (tumor, stroma, immune cells) remain unchanged. In some embodiments, the methods provided herein decrease one or more of soluble galectin-9 levels in blood (serum or plasma), or in galectin-9 tumor tissue expression levels and pattern of expression by immunohistochemistry (tumor, stroma, immune cells) (e.g., e.g., as measured at 2 weeks, 4 weeks, 1 month, 2 month, 3 months, 6 months or 12 months, or at a later time or at any other clinically indicated time point). Galectin-9 levels can be compared to baseline levels prior to initiation of treatment. In some embodiments, the Galectin-9 levels may be compared to healthy subjects. In some embodiments, treating results in a change in PD-L1 expression, e.g., by immunohistochemistry. 16 mg/kg or higher dose level16 mg/kg or higher dose level16 mg/kg or a higher dose level.

In some embodiments, the disclosure provides methods for changing PD-L1 expression, e.g., as assessed by immunohistochemistry (e.g., as measured at 2 weeks, 4 weeks, 1 month, 2 month, 3 months, 6 months or 12 months, or at a later time or at any other clinically indicated time point), comprising administering to the subject a therapeutically effective amount of an anti-Galectin-9 antibody as disclosed herein. In some embodiments of the methods, PD-L1 expression, e.g., as assessed by immunohistochemistry, remains unchanged. PD-L1 levels can be compared to baseline levels prior to initiation of treatment. In some embodiments, the methods provided herein decrease PD-L1 expression, e.g., as assessed by immunohistochemistry. PD-L1 levels may be measured using routine methods known in the art. In one non-limiting example, PD-L1 (SP263) (Roche, Ventana) can be used for detection of PD-L1 in cancer tissues using immunohistochemistry. 16 mg/kg or higher dose level16 mg/kg or higher dose level 16 mg/kg or a higher dose level.

In some embodiments, the disclosure provides methods for changing one or more tumor markers (increasing or decreasing) relevant for the disease (e.g., as measured at 2 weeks, 4 weeks, 1 month, 3 months, 6 months or 12 months, or at a later time or at any other clinically indicated time point), comprising administering to the subject a therapeutically effective amount of an anti-Galectin-9 antibody as disclosed herein. In some embodiments of the methods, one or more tumor markers (increasing or decreasing) relevant for the disease, remain unchanged. Examples of such tumor markers include, but not limited to CA15-3, CA-125, CEA, CA19-9, alpha fetoprotein. Levels of tumor markers can be compared to baseline levels prior to initiation of treatment. In some embodiments, the methods provided herein decrease the occurrence of one or more tumor markers relevant for the disease.

In some embodiments, the disclosure provides methods for changing one or more biomarkers (increasing or decreasing) relevant for the disease (e.g., as measured at 2 weeks, 4 weeks, 1 month, 2 months, 3 months, 6 months or 12 months, or at a later time or at any other clinically indicated time point), comprising administering to the subject a therapeutically effective amount of an anti-Galectin-9 antibody as disclosed herein. Levels of biomarkers in clinical tissues from patients can be measured using routine methods, such as multiplex Immunofluorescence (mIF) technology, as described herein in the examples. An exemplary panel of biomarkers may include CD3, CD4, CD8, CD45RO, FoxP3, CD11b, CD14, CD15, CD16, CD33, CD68, CD163, HLA-DR, Arginase1, Granzyme B, Ki67, PD-1, PD-L1, and PanCK.

### Kits for Use in Treatment of Solid Tumor

The present disclosure also provides kits for use in treating or alleviating a solid tumor such as those disclosed herein (e.g., head and neck cancer or urothelial carcinoma). Such kits can include one or more containers comprising an anti-Galectin-9 antibody, e.g., any of those described herein (e.g., G9.2-17(IgG4)), and a checkpoint inhibitor such as an anti-PD-1 antibody as disclosed herein, e.g., tislelizumab) to be co-used with the anti-Galectin-9 antibody, which is also described herein.

In some embodiments, the kit can comprise instructions for use in accordance with any of the methods described herein. The included instructions can comprise a description of administration of the anti-Galectin-9 antibody, and the anti-PD-1 antibody, to treat, delay the onset, or alleviate a target disease as those described herein. In some embodiments, the kit further comprises a description of selecting an individual suitable for treatment based on identifying whether that individual has the target disease, e.g., applying the diagnostic method as described herein. In still other embodiments, the instructions comprise a description of administering an antibody to an individual at risk of the target disease.

The instructions relating to the use of an anti-Galectin-9 antibody and an anti-PD-1 antibody as disclosed herein generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. In some embodiments, a kit has a sterile access port (for example the container may be an intravenous solution bag or a vial having a stopper pierceable by a hypodermic injection needle). In some embodiments, the container also has a sterile access port (for example the container is an intravenous solution bag or a vial having a stopper pierceable by a hypodermic injection needle). At least one active agent in the composition is an anti-Galectin-9 antibody as those described herein.

Kits may optionally provide additional components such as buffers and interpretive information. Normally, the kit comprises a container and a label or package insert(s) on or associated with the container. In some embodiments, the invention provides articles of manufacture comprising contents of the kits described above.

### General Techniques

The practice of the present invention employs, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook, et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1: Evaluation of Anti-Gal-9 Antibodies Alone or in Combination with Checkpoint Inhibition in a Mouse Model of Pancreatic Cancer and Tumor Mass and Immune Profile of Mice Treated with G9.2-17 mIgG1

The effect of G9.2-17 mIgG1 on tumor weight and on immune profile was assessed in a mouse model of pancreatic cancer. 8-week old C57BL/6 male (Jackson Laboratory, Bar Harbor, ME) mice were administered intra-pancreatic injections of FC1242 PDAC cells derived from Pdx1Cre; KrasG12D; Trp53R172H (KPC) mice (Zambirinis C P, et al., TLR9 ligation in pancreatic stellate cells promotes tumorigenesis. J Exp Med. 2015; 212:2077-94). Tumor cells were suspended in PBS with 50% Matrigel (BD Biosciences, Franklin Lakes, NJ) and 1×10⁵ tumor cells were injected into the body of the pancreas via laparotomy. Mice (n=10/group) received one pre-treatment dose i.p. followed by 3 doses (q.w.) of commercial uGalectin 9 mAb (RG9-1, 200 ug, BioXcell, Lebanon, NH) or G9.2-17 mIgG1 (200 μg), or paired isotype, either G9.2-Iso or rat IgG2a (LTF-2, BioXcell, Lebanon, NH) (200 μg) (one dose per week for three weeks). Mice were sacrificed 3 weeks later and tumors were harvested for analyses by flow cytometry. Tissue was processed and prepared and flow cytometric analysis was performed following routine practice. See, e.g., U.S. Pat. No. 10,450,374.

Tumor Mass and Immune Profile of Mice Treated with G9.2-17 mIgG2a Alone or in Combination with aPD-1 mAb

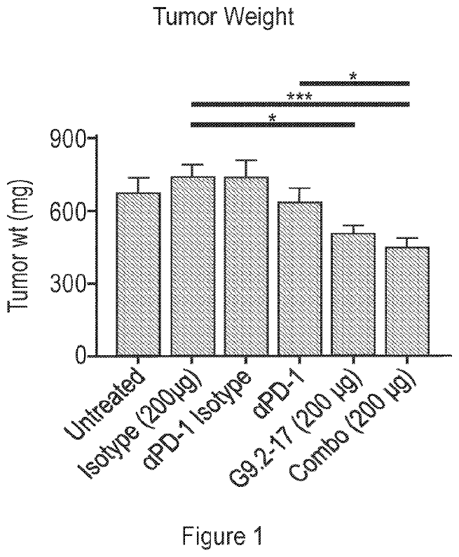

The effect of G9.2-17 mIgG2a on tumor weight and on immune profile was assessed in a mouse model of pancreatic cancer, alone or in combination with immunotherapy. 8-week old C57BL/6 male mice (Jackson Laboratory, Bar Harbor, ME) were administered intra-pancreatic injections of FC1242 PDAC cells derived from Pdx1Cre; KrasG12D; Trp53R172H (KPC) mice. Tumor cells were suspended in PBS with 50% Matrigel (BD Biosciences, Franklin Lakes, NJ) and 1×105 tumor cells were injected into the body of the pancreas via laparotomy. Mice received one pre-treatment dose i.p. followed by 3 doses (q.w.) of G9.2-17 mIgG2a (200p g) or a neutralizing uPD-1 mAb (29F.1A12, 200 μg, BioXcell, Lebanon, NH), separately or in combination, or paired isotype (LTF-2 and C1.18.4, BioXcell, Lebanon, NH) as indicated. Mice were sacrificed on day 26 and tumors were harvested for analyses. Tissue was processed and prepared and flow cytometric analysis was performed following routine practice. See, e.g., U.S. Pat. No. 10,450,374. Each point represents one mouse; *p<0.05; **p<0.01; ***p<0.001; ****p<0.0001; by unpaired Student's t-test. These results show single-agent treatment with G9.2-17 mIgG2a reduces tumor growth at both of the dose levels, whereas anti-PD-1 alone had no effect on tumor size. FIG. 1.

Significant increases in the level of cytotoxic T cells (CD8) are observed in treatments with anti-galectin-9 mIgG1 200 μg+anti-PD-1 (p<0.001) compared to that of anti-galectin-9 mIgG1 200 μg, and between anti-galectin-9 IgG1 200 μg+anti-PD-1 compared to anti-PD-1 alone (p<0.01). Such results suggest that anti-Gal9 antibody and anti-PD-1 antibody in combination would be expected to achieve superior therapeutic effects.

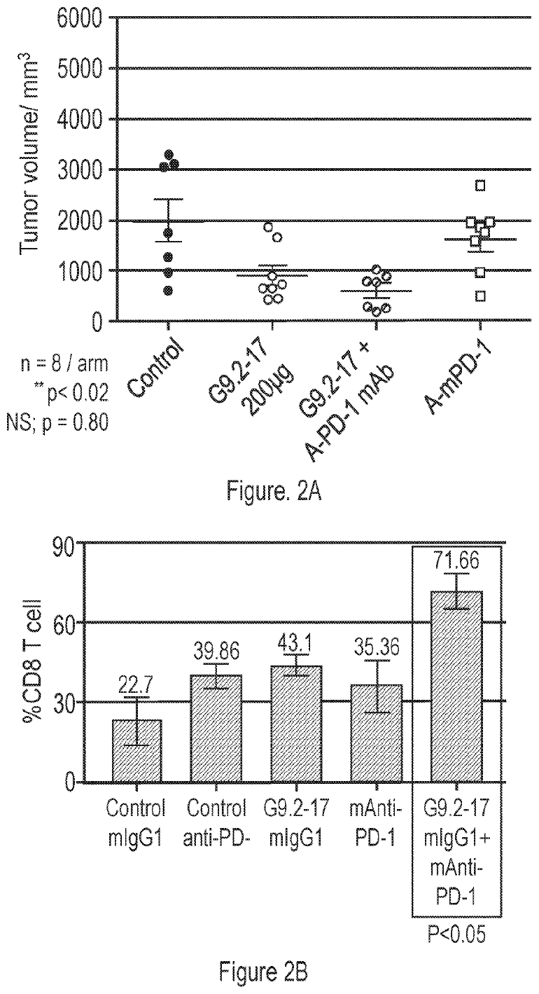

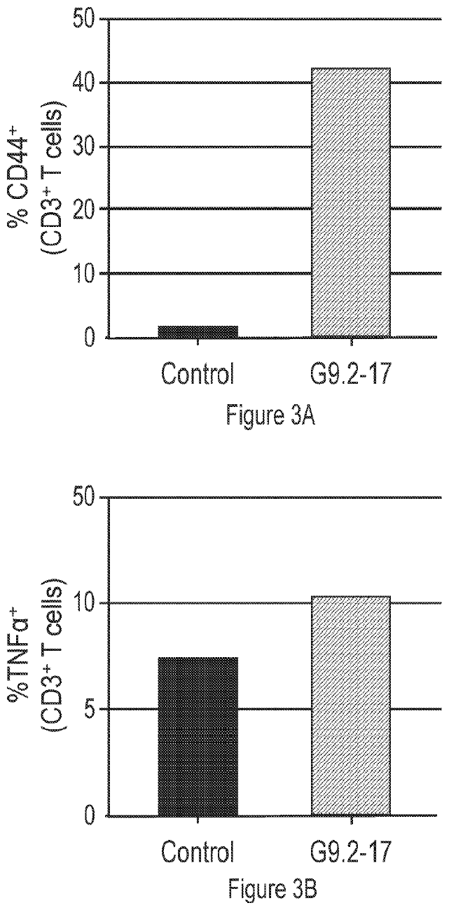

Further, tumor immune responses to treatment with G9.2-17 IgG1 mouse mAb (a.k.a. G9.2-17 mIgG), anti-PD-1 antibody, or a combination of the G9.2-17 IgG1 mouse mAb and anti-PD-1 antibody were investigated in the B16F10 subcutaneous syngeneic model described herein. As shown in FIG. 2A and FIG. 2B, the G9.2-17 and anti-PD-1 combination showed synergistic effects in reducing tumor volume and in increasing CD8+ cells in the mouse model. FIGS. 3A and 3B show that the G9.2-17 antibody increased CD44 and TNFα expression in intratumoral T cells.

### Example 2: Pharmacokinetics of G9.2-17(IgG4) in Human Studies

A Phase 1/2, open-label, multicenter study of safety, pharmacokinetics, and anti-tumor activity of G9.2-17(IgG4) alone or in combination with chemotherapy or immune checkpoint inhibitors (e.g., PD1 antagonists) was performed on patients with metastatic solid tumors. This study is evaluating G9.2-17(IgG4) at the dose levels of 0.2, 0.63, 2.0, 6.3, 10, or 16 mg/kg administered every 2 weeks (Q2W), and 10 and 16 mg/kg every week (QW). See WO2020/223702, WO2022/109302, International Patent Application No. PCT/US2022/027127, and International Patent Application No. PCT/US2022/027142, the relevant disclosures of each of which are incorporated by reference for the subject matter and purpose referenced herein.

Preliminary PK data for 13 subjects from this study were available for analysis. Based on the preliminary data, the beta half-life (i.e., elimination half-life for a 2-compartmental model) of G9.2-17(IgG4) was found to be longer than the non-compartmental analysis (NCA)-derived half-life. These results suggest that the elimination half-life for G9.2-17(IgG4) ranges between 102 and 224 hours (i.e., 4.3 to 9.3 days) over the 0.2 mg/kg to 16 mg/kg dose range, suggesting that weekly dosing of G9.2-17(IgG4) may be an appropriate dosing schedule for this antibody in human treatment.

### Example 3: A Phase 1/2 Open-Label, Multi-Center Study of the Safety, Pharmacokinetics, and Anti-Tumor Activity of G9.2-17(IgG4) as a Single Agent and in Combination with Tislelizumab in Patients with Locally Advanced or Metastatic Solid Tumors

This is an open-label, non-randomized, multi-center, Phase 1/2 study with a dose escalation phase (Part 1) and a cohort expansion phase (Part 2) in patients with relapsed and/or refractory, unresectable locally advanced or metastatic solid tumors. This study will be conducted at up to 20 sites in the United States. The study duration is estimated to be 12-24 months. Follow-up for survival will continue for up to 2 years.

### Treatment Duration

Study drug administration is planned to continue until progression of disease, unacceptable toxicity, or withdrawal from the study. Patients who discontinue the study drug prior to disease progression and are not being treated with other anticancer therapy(ies), will be followed on the study until the time of disease progression.

### Treatment Periods

The study consists of the following periods in both Part 1 and Part 2 (see disclosures below):
Screening period: up to 4 weeks prior to the first dose (Day −28 to Day −1)
Treatment period: 28-day treatment cycles
Post-treatment period: 30 days after the last treatment (End of Treatment Visit/Early Termination Visit)
Immune-Mediated Adverse Reactions (IMAR) follow-up period: All patients treated with G9.2-17 IgG4+tislelizumab must return 90-days+/−7 days after the last dose of study drug for an assessment of potential IMARs.
Follow-up period: Long-term follow-up for up to 2 years (follow-up every 3 months).

### Study Design

### Part 1: Single Agent: Dose Escalation Phase

A dose-finding study will be conducted using a continuous reassessment method (CRM) to establish dose-limiting toxicities (DLTs) and to help evaluate the potential recommended Phase 2 dose (RP2D). Two to 6 patients per treatment Cohorts 1-6 will be assigned to receive sequentially higher intravenous (IV) infusions of G9.2-17 IgG4 every 2 weeks (Q2W) on Day 1 and Day 15 of each 28-day cycle, starting at a dose of 0.2 mg/kg. Patients assigned to a specific dose escalation cohort will receive the corresponding study dose for that cohort. They receive study drug until progression of disease, unacceptable toxicity, or withdrawal from the study for other reasons.

Part 1, Cohorts 1-8 enroll a maximum of approximately 44 patients. A total of 6 dosage levels is to be evaluated:
Dose Escalation Cohort 1=0.2 mg/kg Q2W
Dose Escalation Cohort 2=0.63 mg/kg Q2W
Dose Escalation Cohort 3=2 mg/kg Q2W
Dose Escalation Cohort 4=6.3 mg/kg Q2W
Dose Escalation Cohort 5=10 mg/kg Q2W
Dose Escalation Cohort 6=16 mg/kg Q2W

If the RP2D is not achieved during Cohorts 1-6, an additional 2 dosage levels may be included for the consideration of RP2D:
Dose Escalation Cohort 7=10 mg/kg QW
Dose Escalation Cohort 8=16 mg/kg QW

In Part 1, sequentially increasing doses of G9.2-17(IgG4) are administered by IV infusion either Q2W (Cohorts 1-6) or QW (Cohorts 7-8).

For Cohorts 1-6, 2 patients at a time are to be dosed. Dose escalations may be initiated based on analysis of patient safety data focusing on occurrences of DLTs at previous dose levels and other relevant safety and dosing data from previous cohorts. Dose escalations may occur after a minimum of 28 days (1 cycle).

Following the completion of Cohort 6, a once weekly (QW) G9.2-17 IgG4 dosing schema is to be evaluated, provided the RP2D has not been reached. Patients are to be allowed to enter Cohort 7 when safety data from Cohort 6 is analyzed and no DLT has been identified.

Four patients per dose level in Cohorts 7 and 8 are assigned to receive IV infusions of G9.2-17 IgG4 QW on Days 1, 8, 15, and 22 of each 28-day cycle. Starting with the first 4 patients in Cohort 7, dose escalations to the next cohort occurs if no DLTs are identified.

Those patients treated in early cohorts prior to identification of the RP2D are allowed to dose escalate to the RP2D. They can continue be on the RP2D until they are discontinued from treatment for toxicity, disease progression, or other reasons.

Dose escalations are based on the development of DLTs in patients treated at previous dose levels. For each dose cohort, prior DLT probabilities are to be specified from GLP-compliant toxicity studies as well as from preclinical models. For the specified target DLT rate and total number of dose levels, the skeleton for a power model d\hat{ }exp(a) will be generated according to the approach of Lee and Cheung, using a prior MTD or OBD adjusted by PK/PD data, located at the median dose level and a spacing measure of delta=0.05 (Lee and Cheung, 2011). The prior distribution on the parameter “a” has a mean zero normal distribution with the least informative prior variance. The RP2D is the OBD/MTD dose derived from Part 1.

For patients who experience toxicities (including IMARs) outside of the DLT window, dose reduction is allowed only clinical benefit is being derived and may continue to be derived with lower doses of G9.2-17 IgG4. The dose of G9.2-17 IgG4 will initially be reduced by 50%, and potentially by a further reduction of 50%, as defined by the dose modification guidance provided in the protocol. No further dose reductions will be allowed, See Appendix 8 for detailed instructions on dose modifications.

### Part 1 Combination Treatment: Dose Escalation

For the Part 1 Combination Treatment (Cohorts 9-14 listed below), patients are dosed using a 4+2 design algorithm. Each disease indication (Cohort 9 and 10—Pancreatic ductal adenocarcinoma [PDAC]; Cohorts 11 and 12—Head and Neck [H/N]; Cohorts 13 and 14-Urothelial carcinoma), is conducted in independent cohorts, as follows:
Combination Cohort 9=G9.2-17 IgG4 6.3 mg/kg QW+gemcitabine/nab-paclitaxel (PDAC) (up to n=10)
Combination Cohort 10=G9.2-17 IgG4 16.0 mg/kg QW+gemcitabine/nab-paclitaxel (PDAC) (up to n=10)
Combination Cohort 11=G9.2-17 IgG4 6.3 mg/kg QW+tislelizumab 300 mg every 4 weeks (Q4W) (H/N) (up to n=6)
Combination Cohort 12=G9.2-17 IgG4 16.0 mg/kg QW+tislelizumab 300 mg Q4W (H/N) (up to n=6)
Combination Cohort 13=G9.2-17 IgG4 6.3 mg/kg QW+tislelizumab 300 mg Q4W (Urothelial) (up to n=6)
Combination Cohort 14=G9.2-17 IgG4 16.0 mg/kg QW+tislelizumab 300 mg Q4W (Urothelial) (up to n=6)

Cohorts 9, 11 and 13 (the 6.3 mg/kg lower dose for PDAC, H/N and urothelial, respectively), can run-in parallel. The higher doses of each of these cohorts (16.0 mg/kg) are open to enrollment DLT occurrences and other safety parameters are evaluated.

### Dose De-Escalation

In the event of DLTs (s) during Cycle 1, the following occurs. If, during Cycle 1 of Cohort 9, 1 of 4 patients reaches DLT, then 2 additional patients are to be added to the same Cohort. If 2 or more patients reach DLT out of 4-6 patients in Cohort 9, then Cohort 10 may be opened at a lower dose of G9.2-17 IgG4. The same procedure is to be used for Cohort 11-12 and 13-14, respectively.

For patients who experience toxicities (including IMARs) outside of the DLT window, dose reduction is allowed if clinical benefit is being derived and may continue to be derived with lower doses of G9.2-17 IgG4. The dose of G9.2-17 IgG4 may initially be reduced by 50%, and potentially by a further reduction of 50%, as defined by the dose modification guidance provided in the protocol. No further dose reductions is allowed.

### Part 2: Expansion

Once safety is established in any of the Part 1 cohorts, and a preliminary efficacy and or PD signal is identified, 1 or more expansion cohorts may be launched to further evaluate the safety and efficacy in that particular tumor type. The sample size for each of the expansion arms will be determined based on the point estimates (1) available with standard of care [null hypothesis] versus (2) anticipated with the proposed combination therapy [alternative hypothesis] for each tumor type investigated in Part 1. A protocol amendment may be submitted with details around the expansion population, treatment regimen, and statistical methods prior to initiating Part 2.

In Part 2, patients receive the RP2e of G9.2-17 IgG4 (as determined in Part 1) as a single agent, or the RP2D-1 in combination with PD-1 tislelizumab in patients having head and neck cancer, urothelial cancer, or other solid tumors.

G9.2-17 IgG4. If for any reason same-day administration cannot be accomplished, tislelizumab may be administered on the first day, and G9.2-17 IgG4 on the subsequent day.

### Study Objectives and Endpoints

Part 1 - Dose escalation

| OBJECTIVES | ENDPOINTS |
|---|---|
| Primary | |
| To establish the safety and tolerability of | Evaluation of safety parameters |
| G9.2-17 IgG4 as a single agent treatment | including adverse events, vital sign |
| and in combination with gemcitabine/nab- | measurements, clinical safety laboratory |
| paclitaxel or tislelizumab | tests, 12-lead electrocardiograms (ECG), |
| To determine the recommended Phase 2 | echocardiography/cardiac ultrasound |
| dose (RP2D) for G9.2-17 IgG4 | (ECHO), physical examinations |
| | Evaluation of DLTs |
| | Determination of RP2D |
| Secondary | |
| To characterize the pharmacokinetic (PK) | Evaluation of PK parameters of G9.2-17 |
| profile of G9.2-17 IgG4 | IgG4 (including but not limited to area under |
| | the curve from time zero until 336 h |
| | [AUC_{0-336 h}], maximum observed serum |
| | concentration [Cₘₐₓ], time to reach Cₘₐₓ |
| | [Tₘₐₓ], estimated half-life [_{t1/2}]) |
| To assess the pharmacodynamics (PD) of | Peripheral blood mononucleocyte |
| G9.2-17 IgG4 | (PBMC) immunophenotyping by flow |
| | cytometry |
| | Pre-specified cytokine profile (serum) by |
| | enzyme-linked immunosorbent assay |
| | (ELISA) |
| | Soluble gal-9 levels in blood (serum) by |
| | ELISA |
| | Gal-9 tumor tissue expression levels, and |
| | pattern of expression by |
| | immunohistochemistry (IHC) (expression |
| | on tumor cells and immune cells as well |
| | as a combined score) |
| | Tumor tissue multiplex |
| | immunophenotyping |
| | Programmed death ligand-1 (PD-L1) |
| | expression by IHC |
| | Mismatch repair (MMR) status by IHC |
| To assess the immunogenicity of G9.2-17 | Evaluation of anti-drug antibodies (ADA) |
| IgG4 | |
| Exploratory | |
| To evaluate the preliminary efficacy of | Evaluation of confirmed objective |
| G9.2-17 IgG4 as a single agent treatment | response rate (ORR) |
| and in combination with gemcitabine/nab- | Evaluation of disease control rate (DCR) |
| paclitaxel or tislelizumab | Evaluation of duration of response (DoR) |
| | Evaluation of progression-free survival |
| | (PFS) |
| | Evaluation of overall survival (OS) |

PART 2 - Cohort Expansion

| Objectives | Endpoints |
|---|---|
| Primary | |
| To establish the safety and tolerability of | Evaluation of safety parameters |
| G9.2-17 IgG4 as single treatment and in | including adverse events, vital sign |
| combination with tislelizumab. | measurements, clinical safety laboratory |
| | tests, 12-lead ECG, ECHO, physical |
| | examinations |
| | Evaluation of DLTs |
| To assess the ORR in patients with CRC or | Evaluation of the confirmed ORR-3 in |
| CCA treated with G9.2-17 IgG4 alone, or | patients with CRC or CCA |
| in combination with tislelizumab | |
| To assess PFS in patients with PDAC | Evaluation of PFS-6 in patients with PDAC |
| treated with G9.2-17 IgG4 in combination | |
| with | |
| Secondary | |
| To characterize the pharmacokinetic (PK) | Evaluation of PK parameters of G9.2-17 |
| profile of G9.2-17 IgG4 | IgG4 |
| To assess the pharmacodynamics (PD) of | PBMC immunophenotyping by flow |
| G9.2-17 IgG4 | cytometry |
| | Cytokine profile (serum) by ELISA |
| | Soluble gal-9 levels in blood (serum) by |
| | ELISA |
| | Gal-9 tumor tissue expression levels, and |
| | pattern of expression by IHC (expression |
| | on tumor cells and immune cells as well |
| | as a combined score) |
| | Tumor tissue multiplex |
| | immunophenotyping |
| | PD-L1 expression in tissue by IHC |
| | MMR status in tissue by IHC |
| | TMB in tissue |
| To assess the immunogenicity of G9.2-17 | Evaluation of ADA |
| IgG4 | |
| To evaluate efficacy of G9.2-17 IgG4 | Confirmed ORR |
| | PFS |
| | DCR |
| | DoR |
| | OS |

### Single Agent and Combined Treatment

Treatment of single agent cohorts or combination agent cohorts for solid tumor patients (e.g., head and neck cancer or urothelial carcinoma) may be executed in parallel.

### G9.2-17IgG4 Single Treatment

The starting dose of G9.2-17 IgG4 in the single treatment may be the RP2D identified in Part 1. After testing the investigational drug on 23 patients in Stage I, this trial arm may be terminated if ≤1 patient responds. If the trial goes on to the Stage II of Simon's optimal design, approximately 33 patients are to be treated additionally in each of the single-agent arms. If the total number of responding patients is ≤5, the investigational drug within that arm will be rejected. If ≥6 patients have a confirmed ORR-3, the Part 3 expansion cohort for that arm will be activated and described in an amendment to the protocol.

Dose reduction may be allowed if clinical benefit is being derived and may continue to be derived with lower doses of G9.2-17 IgG4. The dose of G9.2-17 IgG4 may initially be reduced by 50%, and potentially by a further reduction of 50%, as defined by the dose modification guidance provided herein.

### G9.2-17IgG4+Tislelizumab Combination Treatment

The dose of G9.2-17 IgG4 in the combination treatment with tislelizumab may be the RP2D-1, which is the dose immediately preceding the RP2D dose identified in Part 1. The optimal two-stage design may also be used to test the null hypothesis that the ORR-3 is ≤10% versus the alternative hypothesis that the ORR-3 is ≥25%.

To ensure patient safety, a safety run-in will be performed in which the first 8 patients will be dosed. This arm may continue to enroll only if ≤2 patients develop a DLT, which will be below the target toxicity level (TTL) of 25%. If 3 or more patients develop a DLT this combination arm may be terminated for the cancer type being treated. If a DLT occurs, in any of the 8 safety run in patients, during the first 28 days of treatment, that patient may be permanently discontinued from study drug administration.

For patients who experience toxicities outside of the DLT window, dose reduction may be allowed when clinical benefit is being derived and may continue to be derived with lower doses of G9.2-17 IgG4. The dose of G9.2-17 IgG4 may initially be reduced by 50%, and potentially by a further reduction of 50%, as defined by the dose modification guidance provided in the protocol. No further dose reductions may be allowed. Dose modifications for tislelizumab may also be allowed as defined by the guidance herein and Table 7 below.

If an IMAR occurs/recurs that is not managed by dose reduction of either agent, both study medications may be discontinued.

### Dose-Limiting Toxicity (DLT) Criteria

Dose-limiting toxicities assessed in this trial are defined as a clinically significant hematologic and/or non-hematologic AE or abnormal laboratory value assessed as unrelated to metastatic tumor disease progression, intercurrent illness, or concomitant medications and is possibly related or related to the study drug and occurring during the first cycle (28 days) on study. Any patient that experiences a DLT in Part 1 or Part 2 during the first 28 days of treatment will be permanently discontinued from study drug administration.

A DLT is a toxicity that meets any of the following criteria:
Any death not clearly due to the underlying disease or extraneous causes
Indications of potential drug induced liver injury (Hy's Law cases) as follows:
   ALT or AST>3× the upper limit of normal (ULN) with confirmation by repeat testing 24 hours later, AND
   Serum total bilirubin (TBL)>2×ULN with confirmation by repeat testing 24 hours later
   No other explanation can be found for the elevated TBL and/or ATs, such as viral hepatitis (A, B or C), alcoholic or autoimmune hepatitis, pre-existing or acute liver disease, gall bladder obstruction or bile duct disease, Gilbert syndrome, disease progression, or another medication capable of causing the observed effect.
All Grade 4 non-hematologic and hematological toxicities of any duration
All Grade 3 non-hematologic and hematological toxicities. Exceptions are as follow:
   Grade 3 nausea, vomiting and diarrhea that does not require hospitalization or total parenteral nutrition support and can be managed with supportive care to ≤Grade 2 within 48 h.
   Grade 3 electrolyte abnormalities that are corrected to 5 Grade 2 within 24 h.
   Grade 3 electrolyte abnormality that lasts <24-72 hours, is not clinically complicated, and resolves spontaneously or responds to conventional medical interventions.
   ≥Grade 3 amylase or lipase that is not associated with symptoms or clinical manifestations of pancreatitis.

### Statistical Methods:

### Sample Size

Dose escalations will be based on the presence or absence of DLTs in patients treated at previous dose levels. For each dose cohort, prior DLT probabilities are to be specified from Good Laboratory Practice (GLP)-compliant toxicity studies as well as from preclinical models. For the specified target DLT rate and total number of dose levels, the skeleton for a power model d\hat{ }exp(a) will be generated according to the approach of Lee and Cheung (2011), using a prior MTD adjusted by pharmacokinetic (PK)/pharmacodynamic (PD) data, located at the median dose level and a spacing measure of delta=0.05. The prior distribution on the parameter “a” has a mean zero normal distribution with the least informative prior variance. The trial is stopped for safety if the lower limit of an Agresti and Coull binomial CI for the lowest study dose level exceeds the target DLT rate.

CRM trial simulation analyses with 1000 iterations suggested an average of approximately 20 patients will be needed to inform the selection of RP2D, which is the largest dose that has an estimated probability of a DLT less than or equal to a TTL of 25%. The CRM used is based upon the first 6 cohorts, but that itself will not necessarily determine the RP2D, as data from Cohorts 7 to 14 are also be used to determine the RP2D.

A total approximate sample size of 80 patients is anticipated for Part 1 of the study. Backfill will provide for the enrollment of additional patients if deemed necessary.

Part 2 of this study (cohort expansion phase) may adopt a Simon's two-stage optimal design to establish safety and efficacy for LYT-200 in patients with tumor types that demonstrated safety and preliminary efficacy in Part 1. In Part 2, the total sample size may depend on the number of expansion cohorts selected as a result of the safety and efficacy findings in the Part 1 single agent and combination cohorts.

### Randomization Stratification:

This is an open-label study. In Part 1, patients are to be assigned to treatment according to the CRM design of the study. In Part 2, patients are to be assigned to treatment arm, e.g., in line with the inclusion and exclusion criteria.

### Analysis Populations

The intent-to-treat (ITT) population may be defined as those patients who received at least one dose of the study drug, unless otherwise specified. The primary efficacy analyses may be performed for the ITT. Patient disposition may be performed for the ITT.

The Efficacy Population may be defined as all patients in the ITT and having at least one measurable ORR 3 or PFS 6 assessment. This population may be used for a sensitivity analysis.

The per-protocol (PP) Population may be defined as any patient who received at least one full cycle of G9.2-17(IgG4) and without major protocol deviations.

The safety population (SAF) may be defined as all patients who receive at least one dose of the study drug. The safety analyses may be performed for the SAF.

The PK/PD population may be defined as those patients who have received at least one full cycle of G9.2-17(IgG4).

### General Statistical Plan

A database lock and primary analysis may be performed after the last patient has had their primary endpoint event. A final study analysis may be performed after study completion. All analyses may be descriptive.

### Safety Analysis

All safety analyses may be made on the SAF unless otherwise specified and may be analyzed using descriptive statistics

### Efficacy Analysis

Disease response assessed according to RECIST v1.1 may be summarized descriptively for the ITT and PP. A sensitivity analysis may be performed for the Efficacy Populations.

### Pharmacokinetics, Pharmacodynamics, and Immunogenicity

PK, PD, and immunogenicity can be summarized descriptively for the PK/PD population.

### Schedule of Assessments

The Schedule of Assessment in provided in Table 3, except that Study drug administration is as follow: G9.2-17 IgG4 treatment may be administered, on C1D1 and C1D15 on every cycle. In Part 2, tislelizumab may be administered on Day 1 of every cycle on the G9.2-17 IgG4 combination regimen. Study drug may be administered on Days 1, 8 and 15+/−3 days from C2 onwards. All patients treated with G9.2-17 IgG4+tislelizumab must return 90-days+/−7 days after last dose of study drug for an assessment of potential immune-mediated adverse reactions (IMARs).

### Study Population

Patients are eligible for the study after meeting the following inclusion criteria and not meeting any of the exclusion criteria.

### Inclusion Criteria

### Part I

1. Written Informed Consent (mentally competent patient, able to understand and willing to sign the informed consent form)
2. Age ≥18 years, male or non-pregnant female
3. Able to comply with the study protocol, as per Investigator's judgment
4. Histologically confirmed, unresectable locally advanced or metastatic cancer. There are no limits to prior lines of therapies received for the treatment of the cancer condition for which the patient is being enrolled into this study.
   a. For Part 1 combination urothelial carcinoma cohort: histologically or cytologically confirmed diagnosis of unresectable, locally advancer or metastatic urothelial carcinoma of the renal pelvis, ureter, bladder, or urethra (i.e. transitional cell carcinoma).
   b. For Part 1 combination head and neck cancer cohort: histologically confirmed, locally advanced or metastatic SCCHN (oral cavity, oropharynx, hypopharynx, or larynx).

TABLE 3
Schedule of Assessments

| | | Treatment Phase | | | | | | | | | |
|---|---|---|---|---|---|---|---|---|---|---|---|
| | Pre-Dose | Cycle 1 | | | | Cycle 2 | | | Cycle 3 | | |
| | Screening | (28 days) | | | | (28 days) | | | (28 days) | | |
| | Study Day | | | | | | | | | | |
| | −28 to −1 | 1 | 2 | 8 | 15 | 1 | 8 | 15 | 1 | 2 | 8 |
| Cycle Day | | C1D1 | C1D2 | C1D8 | C1D15 | C2D1 | C2D8 | C2D15 | C3D1 | C3D2 | C3D8 |
| +/−days | | | 1 | 1 | 1 | 2 | 1 | 2 | 2 | 1 | 1 |
| allowable | | | | | | | | | | | |
| Study drug | | X | | X | X | X | X | X | X | | X |
| administration A | | | | | | | | | | | |

Study Procedures & Examinations

| | | | | | | | | | | |
|---|---|---|---|---|---|---|---|---|---|---|
| Eligibility | X | | | | | | | | | |
| assessment and | | | | | | | | | | |
| written informed | | | | | | | | | | |
| consent | | | | | | | | | | |
| Demographics B | X | | | | | | | | | |
| Medical history C | X | | | | | | | | | |
| Previous and | X | X | X | X | X | X | X | X | X | X |
| concomitant | | | | | | | | | | |
| medications D | | | | | | | | | | |
| Adverse events E | | X | X | X | X | X | X | X | X | X |
| ECHO/MUGA F | X | | | | | | | | | |
| 12-lead ECG | X | X | | | | X | | X | | |
| (QTcF) | | | | | | | | | | |
| Physical exam G | X | X | | | X | X | X | X | | |
| ECOG | X | X | | | X | X | X | X | | |
| Vital SignsH | X | X | | | X | X | X | X | | |
| Tumor imaging | X | | | | | | | X | | |
| assessment (CT | | | | | | | | | | |
| with contrast | | | | | | | | | | |
| preferred) N | | | | | | | | | | |

Clinical Labs

| | | | | | | |
|---|---|---|---|---|---|---|
| Pregnancy test I | X | | | X | | X |
| Hematology J | X | X | X | X | X | X |
| Serum | X | X | X | X | X | X |
| Chemistry K | | | | | | |
| TSH, Free T4, | X | X | | X | | X |
| serum lipase, | | | | | | |
| amylase, PTH, | | | | | | |
| FSH, LH, free | | | | | | |
| cortisol | | | | | | |
| Blood | X | X | X | X | X | X |
| Coagulation L | | | | | | |
| Urinalysis M | X | X | X | X | X | X |

Pharmacodynamics and Pharmacokinetics

| | | | | | | | | | | |
|---|---|---|---|---|---|---|---|---|---|---|
| Tumor biopsy O | X | | | | | | | | | |
| Tumor type- | X | X | | | X | X | X | X | | |
| relevant | | | | | | | | | | |
| biomarkers P | | | | | | | | | | |
| Blood sampling | | X | X | X | X | X | | X | X | X |
| for biomarker | | | | | | | | | | |
| PD analysis Q | | | | | | | | | | |
| PK blood | | X | X | X | X | X | | X | X | X |
| sampling R | | | | | | | | | | |
| Immunogenicity | | X | | | | X | | X | | |
| (ADA) S | | | | | | | | | | |
| Survival | | | | | | | | | | |
| Follow-up | | | | | | | | | | |

| | | | | | Post Treatment | | |
|---|---|---|---|---|---|---|---|
| | | | | | | | Long |
| | | | | | | | Term |
| | Treatment Phase | | | | End of | | Follow- |
| | | Cycle 4 | | | Treatment | IMAR | Up U |
| | Cycle 3 | (28 days) and | | | or Early | Follow- | Every |
| | (28 days) | beyond | | | Termination | UpT | 3 mons |
| | Study Day | | | | | | |
| | | | | | 30 days | 90 days | |
| | | | | | after | after | |
| | 15 | 1 | 8 | 15 | last dose | last dose | |
| Cycle Day | C3D15 | CXD1 | CXD8 | CXD15 | — | — | — |
| +/−days | 2 | 2 | 1 | 2 | 3 | 7 | n/a |
| allowable | | | | | | | |
| Study drug | X | X | X | X | | | |
| administration A | | | | | | | |
| Study Procedures & Examinations | | | | | | | |
| Eligibility | | | | | | | |
| assessment and | | | | | | | |
| written informed | | | | | | | |
| consent | | | | | | | |
| Demographics B | | | | | | | |
| Medical history C | | | | | | | |
| Previous and | X | X | | X | X | | |
| concomitant | | | | | | | |
| medications D | | | | | | | |
| Adverse events E | X | X | | X | X | X | |
| ECHO/MUGA F | | X | | | | | |
| 12-lead ECG | | X | | | | | |
| (QTcF) | | | | | | | |
| Physical exam G | X | X | | X | X | X | |
| ECOG | X | X | | X | X | | |
| Vital SignsH | X | X | | X | X | | |
| Tumor imaging | | | | | X | | X U |
| assessment (CT | | | | | repeat if end of | | |
| with contrast | | | | | study is >8 | | |
| preferred) N | | | | | weeks after | | |
| | | | | | last scan | | |
| Clinical Labs | | | | | | | |
| Pregnancy test I | | X | | | X | | |
| Hematology J | X | X | | X | X | X | |
| Serum | X | X | | X | X | X | |
| Chemistry K | | | | | | | |
| TSH, Free T4, | | X | | | X | X | |
| serum lipase, | | | | | | | |
| amylase, PTH, | | | | | | | |
| FSH, LH, free | | | | | | | |
| cortisol | | | | | | | |
| Blood | X | X | | X | X | X | |
| Coagulation L | | | | | | | |
| Urinalysis M | X | X | | X | X | X | |
| Pharmacodynamics and Pharmacokinetics | | | | | | | |
| Tumor biopsy O | X ± 7 d | | | | | | |
| Tumor type- | X | X | | X | X | | |
| relevant | | | | | | | |
| biomarkers P | | | | | | | |
| Blood sampling | X | X | | | X | | |
| for biomarker | | | | | | | |
| PD analysis Q | | | | | | | |
| PK blood | X | X | | | | | |
| sampling R | | | | | | | |
| Immunogenicity | | X | | | X | | |
| (ADA) S | | | | | | | |
| Survival | | | | | | | X U |
| Follow-up | | | | | | | |

ADA: anti-drug antibodies; AE: adverse event; ALT: alanine aminotransferase; APTT: activated partial thromboplastin time; AST: aspartate aminotransferase; C: cycle; CPK: creatine phosphokinase; COVID19: Coronavirus SARS-CoV-2; CRP: C-reactive protein; CT: computed tomography; D or d: day(s); ECG: electrocardiogram; ECOG: Eastern Cooperative Oncology Group; ECHO: echocardiography/cardiac ultrasound; FSH: follicle-stimulating hormone; IMAR: immune-mediated adverse reaction; INR: international normalized ratio; LDH: lactate dehydrogenase; LH: luteinizing hormone; min: minute(s); MUGA: multigated acquisition scan; PD: pharmacodynamics; PK: pharmacokinetics; PT: prothrombin time; PTH: parathyroid hormone; PTT: partial thromboplastin time; QTcF: QT interval, Fridericia's Correction Formula; RBC: red blood cell count; SGOT: serum glutamic-oxaloacetic transaminase; SGPT: serum glutamic pyruvic transaminase; TSH: thyroid stimulating hormone; WBC: white blood cell count.
A) Study drug administration: G9.2-17 IgG4 treatment will be administered, on C1D1 and C1D15 on every cycle.
B) Demographics: Data include age, gender, race, and ethnicity.
C) Medical history: In addition to general medical history, data collection also includes oncology history, surgical/transplant and radiation therapy history and COVID-19 history and testing.
D) Previous and concomitant medications (including vaccines and complementary treatments/supplements): Data to include name, indication, dose, route, start and end dates for each. Allergies and intolerances, dose modifications while on study, schedule of dosing changes and reasons for them should also be obtained.
E) Adverse events: Any AEs starting or worsening after study drug administration will be recorded. AEs should be followed until resolved to one of the following: baseline, stabilized, or deemed irreversible. All SAEs are to be collected until 30 days after last dose of study medication. All study-procedure-related SAEs must be collected from the date of patient's written consent.
F) ECHO/MUGA: This assessment of heart function is conducted at Screening and repeated on Day 1 of Cycle 4; the assessment window is +/−5 days. It should be conducted more frequently when clinically indicated and once every 3 months.
G) Physical exam: Include height at screening for determination of body surface area. Include weight at all scheduled exam times. A Neurological exam will be conducted only on patients who have stable and/or pre-treated brain metastases.
HVital Signs: temperature, heart rate, blood pressure, respiratory rate.
I) Pregnancy test (blood or urine): Only for women of childbearing potential with uterus in situ. Test results must be available before scheduled dosing.
J) Hematology: Analysis includes complete blood count, differential, platelets, hemoglobin. Collect blood samples pre-dose.
K) Serum chemistry: Analysis includes albumin, alkaline phosphatase, bilirubin (total, direct), blood urea nitrogen, calcium, CPK, creatinine, electrolytes (sodium, potassium, chloride, magnesium, phosphorus), gamma glutamyl transferase (gamma GT), glucose, hemoglobin A1c (HgbA1c) (only if history of Type 1 or Type 2 diabetes mellitus), LDH, SGPT (ALT) or SGOT (AST), total protein. Fasting glucose to be assessed only if clinically indicated. Collect blood samples pre-dose.
L) Blood Coagulation: Collect blood samples pre-dose. Analysis includes APTT, PT, PTT, and INR (if on allowable anti-coagulants), CRP, and troponin.
M) Urinalysis: Analysis includes color, appearance, dipstick for specific gravity, protein, white blood cell-esterase, glucose, ketones, urobilinogen, nitrite, WBC, RBC, pH. (Urine culture and sensitivity to be run only if patient is clinically symptomatic.)
N) Tumor imaging assessment: For screening, the assessment must be performed within the 28-day screening period. On study, assessments are done every 8 weeks ± 7 days (ie, C3D1, C5D1, C7D1, C9D1, etc.) and at the End of Treatment if not assessed within the previous 4-6 weeks. Assessments may be performed more frequently if clinically indicated. If an objective response is seen on a scan, a confirmation scan will be done 4 weeks (+7 d) later. After this confirmatory scan, the scheduled scans are to be resumed at a frequency of every 8 weeks ± 7 days from the date of the confirmatory scan.
O) Tumor biopsies: If patient MMR/MSI status is unknown at screening, the test should be run at the local laboratory. In Part 2, TMB tissue analysis will be performed. The on-study biopsy is scheduled for C3D15 ± 7 days and should occur only after the tumor imaging scan in Cycle 3. It is recognized that a variety of clinical factors may make it difficult to obtain adequate specimens. Decisions not to perform biopsy on-treatment should be discussed with the Medical Monitor.
P) Tumor type-relevant biomarkers: Blood samples are to be collected at screening and every cycle pre-dose administration as appropriate for the tumor type. Blood sampling may be decreased to every 3rd cycle after 6 months of treatment.
Q) PD blood sampling: Blood samples will be collected pre-dose administration on dosing days. May be decreased to every 3rd cycle after 6 months of treatment.
R) PK blood sampling: Cycle 1 and Cycle 3 Day 1: blood samples will be collected pre-dose and at end of study drug infusion (EOI), 2 and 4 h (±30 min) post-study drug administration. Cycle 1 and Cycle 3 Day 15, blood samples will be collected pre-dose and at EOI only. Cycle 1 and Cycle 3 Day 2 and 8 (non-dosing days), PK blood samples will be collected at only one time point. Cycle 2 and Cycle 4: blood samples will be collected Day 1 only and should occur pre-dose and at EOI. Blood samples for PK will be collected every 2 cycles thereafter (ie, C6D1, C8D1, etc.) pre-dose and at EOI.
S) ADA blood sampling: Blood samples will be collected Day 1 of Cycles 1-4, pre-dose. Thereafter, it will be collected every 2 cycles, Day 1, pre-dose (ie, C6D1, C8D1, etc.).
T) All patients treated with G9.2-17 IgG4 + tislelizumab must return 90-days +/− 7 days after last dose of study drug for an assessment of potential immune-mediated adverse reactions (IMARs).
U) Long-Term Follow-up: Tumor imaging should continue, where possible, for patients discontinuing treatment due to reasons other than progression of disease and not receiving additional systemic anticancer treatments. Survival data will be collected at a minimum every 3 months. It can be collected more frequently to support data cleaning or regulatory submission efforts. Follow-up can be conducted by telephone, electronic messaging or chart review and will continue for up to 2 years after the patient has the End of Treatment/Early Termination visit.

5. For urothelial and head and neck combination cancer cohorts, prior exposure to immunotherapy is allowed, with standard of care treatment options and/or within a clinical trial context. If the patient received an anti-PD-1 and/or an anti-PD-L1 containing regimen at any point, they must have demonstrated at least stable disease, as per RECIST 1.1. or iRECIST criteria to one of these treatment regimens, if these measurements are available. If RECIST or iRECIST measurements are not available, then clinical PFS of at least 4 months is required to have been achieved on any of the prior anti-PD-1 and/or anti-PD-L1 containing regimens.
6. There is no PD-L1 expression requirement for the Part 1 combination urothelial and head and neck cohorts, however fresh biopsy or archival tissue is required for assessment of PD-L1 by IHC, or a historical PD-L1 expression by IHC must be available. If PD-L1 expression data is already available, this does not override the protocol preference for obtaining a fresh biopsy whenever feasible.
7. For Part 1 combination cohort head and neck cancer patients of oropharynx origin: human papilloma virus (HPV) status needs to be established in the screening period or at any point while patient is on study drug, unless it is historically known. pi6+ as a surrogate for HPV+, HPV RNA ISH or DNA PCR are all acceptable. The study accepts both HPV+ and HPV− patients.
8. Life expectancy >3 months according to Investigator's judgment.
9. ECOG performance status 0-1.
10. Patient able and willing to undergo pre- and on/post treatment biopsies. According to the Investigator's judgment, the planned biopsies should not expose the patient to substantially increased risk of complications. Every effort will be made that the same lesion is biopsied on repeat biopsies. If the patient is eligible according to all other criteria but declines to consent to a biopsy or there are other medical reasons precluding biopsy, this will be discussed with the Sponsor.
11. Measurable disease, according to RECIST v1.1. Note that lesions intended to be biopsied should not be target lesions.
12. Adequate hematologic and end organ function, defined by the following laboratory results obtained prior to first dose of study drug treatment, provided no anticancer treatment was administered within the last 7 days:
   a. neutrophil count ≥1×10⁹/L
   b. platelet count ≥100×10⁹/L; for hepatocellular carcinoma (HCC) in Part 1≥50×10⁹/L
   c. hemoglobin ≥9.0 g/dL without transfusion in the previous week
   d. creatinine ≤1.5×ULN; or eGFR>50 mg/mmol
   e. aspartate aminotransferase AST (SGOT)≤3×ULN (≤5×ULN when HCC or hepatic metastases are present)
   f. alanine aminotransferase (ALT [SGPT])≤3×ULN (≤5×ULN when HCC or hepatic metastases present)
   g. bilirubin ≤1.5×ULN (patients with known Gilbert's disease may have a bilirubin ≤3.0×ULN)
   h. albumin ≥3.0 g/dL
   i. international normalized ratio (INR) and partial thromboplastin time (PTT) ≤1.5×ULN, unless patient receiving anticoagulant therapy.
13. No evidence of active serious infection or infections requiring parenteral antibiotics.
14. Women of childbearing potential must have a negative pregnancy test within 72 h prior to start of treatment. For women of childbearing potential: agreement to remain abstinent (refrain from heterosexual intercourse) or to use contraceptive methods that result in a failure rate of <1% per year during the treatment period and for at least 180 days after the last study treatment.

A woman is of childbearing potential if she is post-menarche, has not reached a postmenopausal state (≥12 continuous months of amenorrhea with no identified cause other than menopause), and has not undergone surgical sterilization (removal of ovaries and/or uterus).

Examples of contraceptive methods with a failure rate of <1% per year include bilateral tubal ligation, male sterilization, hormonal contraceptives that inhibit ovulation, hormone-releasing intrauterine devices and copper intrauterine devices. The reliability of sexual abstinence should be evaluated in relation to the duration of the clinical trial and the preferred and usual lifestyle of the patient. Periodic abstinence (e.g., calendar, ovulation, symptom-thermal, or post ovulation methods) and withdrawal are not acceptable methods of contraception. Fertile men must practice effective contraceptive methods during the study, unless documentation of infertility exists.
15. Four (4) weeks or 5 half-lives (whichever is shorter) since the last dose of anticancer therapy before the first G9.2-17(IgG4) administration.
16. Bisphosphonate treatment (e.g., zoledronic acid) or denosumab are allowed if previously used prior to commencement of clinical trial.
17. Patients:
   a. who have already received at least one prior line of systemic therapy for metastatic or locally advanced disease, and/or
   b. who have a tumor type for which there are no available standard of care options.
18. Patients who have not previously received a gemcitabine-containing regimen.

### Exclusion Criteria

1. Patient unwilling or unable to follow protocol requirements
2. Patient diagnosed with metastatic cancer of an unknown primary
3. Current illicit drug addiction (medical and recreational marijuana/cannabidiol [CBD]/tetrahydrocannabinol [THC] would not be considered “illicit”)
4. Clinically significant, active uncontrolled bleeding, and any patients with a bleeding diathesis (e.g., active peptic ulcer disease). Prophylactic or therapeutic use of anticoagulants is allowed.
5. Pregnant and/or lactating females
6. Receiving any other investigational agents or participating in any other clinical trial involving another investigational agent for treatment of solid tumors within 3 weeks or 5 half-lives of the administered drug (whichever is shorter) prior to the first dose of study drug, or major surgery or planned surgery within 4 weeks of the first dose of study drug (this includes dental surgery).
7. Radiation therapy within 4 weeks of the first dose of study drug, except for palliative radiotherapy to a limited field, such as for the treatment of bone pain or a focally painful tumor mass, and which does not jeopardize required measurable lesions for response assessment (RECIST v1.1).
8. Patients with fungating tumor masses
9. History or current evidence of any condition, therapy, any active infections, or laboratory abnormality that might confound the results of the trial, interfere with the patient's participation for the full duration of the trial, or is not in the best interest of the patient to participate, in the opinion of the treating Investigator
10. Grade 4 immune-mediated toxicities with a prior checkpoint inhibitor. Grade 2 or Grade 3 pneumonitis or any other Grade 3 checkpoint inhibitor-related toxicity that led to immunotherapy treatment discontinuation. Low-grade (<Grade 3) toxicities, such as neuropathy from prior treatments, manageable electrolyte abnormalities and lymphopenia, alopecia and vitiligo are allowed.
11. history of other prior or other concomitant malignancy that requires other active treatment.
12. Active brain, patients with carcinomatous meningitis or leptomeningeal metastases. Patients with brain metastases are eligible provided they have shown clinically and radiographically stable disease for at least 4 weeks after definitive therapy and have not used steroids (>10 mg/day of prednisone or equivalent) for at least 4 weeks prior to the first dose of study drug
13. Evidence of severe or uncontrolled systemic diseases, congestive heart failure >New York Heart Association (NYHA) class 2, myocardial infarction (MI) within 6 months, or laboratory finding that in the view of the Investigator makes it undesirable for the patient to participate in the trial
14. Any medical condition that the Investigator considers significant to compromise the safety of the patient or that impairs the interpretation of LYT-200 toxicity assessment
15. Serious non-healing wound, active ulcer, or untreated bone fracture unless for e.g., a rib fracture for (which does not elicit treatment)
16. Uncontrolled pleural effusion, pericardial effusion, or ascites requiring recurrent drainage procedures. For the purposes of this study, “recurrent” is defined as ³3 drains in the last 30 days.
17. History of severe allergic, anaphylactic, or other hypersensitivity reactions to chimeric or humanized antibodies or fusion proteins
18. Significant vascular disease (e.g., aortic aneurysm requiring surgical repair or recent arterial thrombosis) within 6 months of Cycle 1, Day 1
19. History of pulmonary embolism, stroke or transient ischemic attack within 3 months prior to Cycle 1, Day 1
20. Active autoimmune disorder (except type I/II diabetes, hypothyroidism requiring only hormone replacement, vitiligo, psoriasis, or alopecia areata).
21. Requires systemic immunosuppressive treatment, including, but not limited to cyclophosphamide, azathioprine, methotrexate, thalidomide, and anti-tumor necrosis factor (anti-TNF) agents. Patients who have received or are receiving acute, low dose systemic immunosuppressant medications (e.g., ≤10 mg/day of prednisone or equivalent) may be enrolled. Replacement therapy (e.g., thyroxine, insulin, physiologic corticosteroid replacement therapy [e.g., ≤10 mg/day of prednisone equivalent] for adrenal or pituitary insufficiency) is not considered a form of systemic treatment. The use of inhaled corticosteroids and mineralocorticoids (e.g., fludrocortisone), topical steroids, intranasal steroids, intra-articular, and ophthalmic steroids is allowed
22. Severe tumor-related pain (Grade 3, CTCAE] v.5.0 unresponsive to broad analgesic interventions (oral and/or patches)
23. Hypercalcemia (defined as 3 Grade 3, per CTCAE v 5.0) despite use of bisphosphonates
24. Any other diseases, metabolic dysfunction, physical examination finding, or clinical laboratory finding giving reasonable suspicion of a disease or condition that contraindicates the use of an investigational drug or that may affect the interpretation of the results or render the patient at high risk of treatment complications
25. Received organ transplant(s)
26. Patients undergoing dialysis
27. For Part 1, hormonal androgen deprivation therapy is allowed to continue for patients with metastatic castration-resistant prostate cancer
28. Any ablative therapy (Radio Frequency Ablation or Percutaneous Ethanol Injection) for HCC<6 weeks prior trial entry
29. Hepatic encephalopathy or severe liver adenoma
30. Child-Pugh score ≥7

### Study Drug and Other Interventions

Study intervention(s) is/are defined as any investigational agent(s), marketed product(s), placebo, or medical device(s) intended to be administered/used to/in a study participant according to the study protocol.

Agents Administered in Combination with G9.2-17IgG4

### Tislelizumab

Tislelizumab is a PD-1 inhibiting mAb drug being developed for the treatment of cancer. Tislelizumab is formulated for IV injection in a single-use glass vial (20R glass, USP type I) with a rubber stopper containing a total of 100 mg of tislelizumab mAb in 10 mL of buffered isotonic solution. Tislelizumab is administered as an intravenous infusion over approximately 30 minutes (unless guided otherwise) at 300 mg every 4 weeks, in a 28-day cycle.

The active ingredient of tislelizumab is a humanized IgG4 variant mAb against PD-1, binding to the ECD of human PD-1 with high specificity and affinity (KD=0.15 nM). The excipients of tislelizumab include: sodium citrate dihydrate, citric acid monohydrate, L-histidine hydrochloride monohydrate, L-histidine, trehalose dihydrate, polysorbate-20, and WFI. Tislelizumab competitively blocks the binding of both PD-L1 and PD-L2, inhibiting PD-1-mediated negative signaling and enhancing the functional activity in T cells in in vitro cell-based assays. In addition, tislelizumab demonstrated antitumor activity in several human cancer allogeneic xenograft models and a human PD-1 transgenic mouse model.

The IgG4 variant antibody has very low binding affinity to Fcγ RIIIA and C1q by in vitro assays, suggesting a low or no ADCC and CDC effect in humans. Unlike natural IgG4 antibody, tislelizumab has no observable Fab-arm exchange activity by the in vitro assay, predicting the antibody would be stable in vivo, unlikely forming bispecific antibodies.

Exposure-response (E-R) relationships between tislelizumab exposure and efficacy across a variety of advanced solid tumors support the 300 mg Q4W regimens. 300 mg Q4W regimen is not expected to be clinically different from the 200 mg Q3W in terms of safety or efficacy outcomes.

The safety profile of tislelizumab is consistent with the therapeutic class of the drug with a relatively low rate of treatment-related Grade 3 or above toxicity.

Tislelizumab AEs are presented below in Table 4 according to their frequency of occurrence. Reported AEs that may be IMAR-related are summarized in Table 5.

TABLE 4
Adverse Events (non-IMAR-Related) Reported
for Tislelizumab According to Frequency

| ORGAN SITE | ADVERSE EVENT |
|---|---|
| blood and lymphatic | anemia (8-21%, severe 1-8%) |
| system/ febrile | leukopenia (5-12%, severe 2%) |
| neutropenia | lymphopenia (<1%) |
| | neutropenia (3-9%, severe 1-3%) |
| | thrombocytopenia (1-9%, severe 1%) |
| gastrointestinal | emetogenic potential: low³ |
| | constipation (13%, severe <1%) |
| | diarrhea (5-13%, severe 1%) |
| | nausea (4-13%, severe <1%) |
| | stomatitis, aphthous ulcer (3%, severe <1%) |
| | vomiting (10%, severe 1%) |
| general disorders | extravasation hazard: none⁴ |
| andadministration | chills (1%, severe 0%) |
| site conditions | fatigue, asthenia (1-23%, severe 2%) |
| | pyrexia (5-33%, severe 1%) |
| infections and | pneumonia (3%) |
| infestations | |
| injury, poisoning, | infusion-related reactions (2-29%, severe <2%) |
| and procedural | |
| complications | |
| investigations | alkaline phosphatase increase (6%, severe 1%) |
| | ALT increase (1-16%, severe 2%) |
| | AST increase (1-17%, severe 3%) |
| | bilirubin increase (5-12%, severe 2%) |
| | creatine phosphokinase increase (1-6%, severe 1%) |
| | hyperglycemia (2-5%, severe 1%) |
| | hyponatremia (1%, severe 1%) |
| | lipase increase (1%, severe 1%) |

Clinically important side effects are in bold, italics

| | |
|---|---|
| | proteinuria (1-6%) |
| | thyroid hormone (free triiodothyronine and thyroxine) decrease (1%) |
| | thyroid stimulating hormone (TSH) increase (1-7%) |
| metabolism and | appetite decrease (1-15%, severe <1%) |
| nutrition | diabetes mellitus, type 1 (1%, severe 1%) |
| | diabetic ketoacidosis (<1%) |
| musculoskeletal | arthralgia (2-6%) |
| andconnective | |
| tissue | |
| respiratory, thoracic | cough (13%) |
| andmediastinal | dyspnea (6%, severe 1%) |
| | pruritus (8-14%, severe <1%) |
| | rash (6-18%, severe 1%) |
| | skin reaction, undefined (1%, severe <1%) |

By disrupting PD-1-mediated signaling, tislelizumab acts to restore antitumor immunity and halt progression of tumor growth. This restoration of immune system activity may result in immune related adverse reactions involving 1 or more body systems, which can be life threatening or fatal in rare cases. While these events usually become manifest during treatment with tislelizumab, they can also occur after discontinuation of tislelizumab therapy.

TABLE 5
Adverse Events Reported for Tislelizumab to be Treated as IMARs*

| ORGAN SITE | ADVERSE REACTION |
|---|---|
| cardiac | immune-mediated myocarditis (1%, severe <1%) |
| endocrine | immune-mediated adrenal insufficiency (<1%) |
| | immune-mediated hyperthyroidism (3-4%, severe <1%) |
| | immune-mediated hypothyroidism (6-21%, severe 1%) |
| | immune-mediated thyroiditis (1%) |
| eye | immune-mediated uveitis (<1%) |
| gastrointestinal | immune-mediated colitis (1%, severe 1%) |
| | immune-mediated pancreatitis (1%, severe 1%) |
| | immune-mediated hepatitis (1-3%, severe 1-2%) |
| musculoskeletal | arthralgia (2-6%) |
| andconnective | immune-mediated myositis/rhabdomyolysis (1%, severe 1%) |
| tissue | immune-mediated nephritis, renal dysfunction (1%, severe 1%) |
| immune system | immune-mediated adverse events (21-22%, severe 5-11%) |
| respiratory, thoracic | cough (13%) |
| and mediastinal | dyspnea (6%, severe 1%) |
| | immune-mediated pneumonitis, interstitial lung disease |
| | (2-7%, severe 1-6%) |
| skin and | immune-mediated dermatitis (1%, severe <1%) |
| subcutaneous tissue | immune-mediated pruritus (2-4%) |
| | immune-mediated rash (3%, severe <1%) |
| | immune-mediated vitiligo (1%) |

See also Table 6 below for management of IMARs caused by the combination of G9.2-17(IgG4) and tislelizumab.

TABLE 6
Management of Immune-Mediated Adverse Reactions (IMARs) Caused
by G9.2-17 IgG4 + Tislelizumab Combination Treatment

| IMAR | Guidance for Management |
|---|---|
| Immune-mediated pneumonitis | Monitor patients for signs with radiographic imaging |
| | and for symptoms of pneumonitis. |
| | Grade 1, consider holding tislelizumab until there is |
| | significant improvement in patient's signs and |
| | symptoms |
| | Withhold tislelizumab and G9.2-17 IgG4 for moderate |
| | (Grade 2) and permanently discontinue for severe |
| | (Grade 3) or life-threatening (Grade 4) pneumonitis. |
| | Administer corticosteroids at a dose of 1 to 2 |
| | mg/kg/day prednisone equivalents for moderate (Grade |
| | 2). For more severe (Grade 3-4) pneumonitis, |
| | permanently discontinue G9.2-17 IgG4 and |
| | tislelizumab and treatment in a hospital setting with IV |
| | methylprednisolone 2 to 4 mg/kg/day is recommended, |
| | followed by corticosteroid taper. |
| | Withhold tislelizumab and G9.2-17 IgG4 until |
| | resolution to <Grade 2 for moderate (Grade 2) |
| | pneumonitis. Resume G9.2-17 IgG4 at a 50% dose |
| | reduction. |
| | Tislelizumab may be restarted only if symptoms |
| | resolve completely or are controlled on prednisolone ≤10 |
| | mg/day. Withhold tislelizumab for Grade 2 |
| | pneumonitis, and only restart if symptoms resolve |
| | completely or are controlled on prednisolone ≤10 |
| | mg/day. Permanently discontinue treatment with |
| | tislelizumab if symptoms persist with the use of |
| | corticosteroids. |
| | Upon recurrence of ≥Grade 2 pneumonitis, |
| | permanently discontinue treatment with both the study |
| | medication and tislelizumab |
| Immune-mediated colitis and | Monitor patients for signs and symptoms of colitis. |
| diarrhea | Withhold treatment with tislelizumab and G9.2-17 |
| | IgG4 for Grade 2 diarrhea or colitis and start oral |
| | prednisolone 0.5 mg/kg/day (nonenteric coated). If |
| | worsening or no improvement occurs despite initiation |
| | of corticosteroids, increase dose to 1 to 2 mg/kg/day |
| | prednisone equivalents. |
| | Taper steroids over 2 to 4 weeks with resolving signs |
| | and symptoms. Resume treatment with tislelizumab |
| | when signs and symptoms improve or resolve to |
| | baseline. Administer prednisolone 1 to 2 mg/kg/day for |
| | colitis Grade 3 or Grade 4, converting to oral |
| | prednisolone and tapering this dose over at least 4 |
| | weeks when improvement is evident. Permanently |
| | discontinue tislelizumab and G9.2-17 IgG4 for |
| | recurrent severe (Grade 3) or life-threatening Grade 4 |
| | diarrhea or colitis. |
| | Resume G9.2-17 IgG4 at a 50% dose reduction when |
| | signs and symptoms improve or resolve to baseline. |
| | Any dose modifications for tislelizumab are at the |
| | Investigator's discretion. |
| | If ≥Grade 3 recurs, then permanently discontinue |
| | treatment with both the study medication and |
| | tislelizumab. |
| | If ≥Grade 2 colitis recurs, withhold G9.2-17 IgG4 and |
| | tislelizumab until colitis resolves to baseline. Apply |
| | treatment regimen outlined above. Resume G9.2-17 |
| | IgG4 at an additional 50% dose reduction. No further |
| | dose reductions for G9.2-17 IgG4 are allowed. |
| | If ≥Grade 2 colitis recurs, permanently discontinue |
| | treatment with both study medications. |
| | Permanently discontinue G9.2-17 IgG4 and |
| | tislelizumab for life-threatening (Grade 4). |
| Immune-mediated hepatitis | Monitor for changes in liver function. |
| | Administer corticosteroids at a dose of 0.5 to 1 |
| | mg/kg/day prednisone equivalents for moderate (Grade |
| | 2) transaminase elevations. |
| | Withhold tislelizumab and G9.2-17 IgG4 for moderate |
| | (Grade 2) immune-mediated hepatitis. When resolved |
| | to <Grade 2, and prednisolone tapered to ≤10 mg, |
| | resume tislelizumab and G9.2-17 IgG4. G9.2-17 IgG4 |
| | is to be resumed at a 50% dose reduction. |
| | If ≥Grade 2 hepatitis recurs, permanently discontinue |
| | treatment with both G9.2-17 IgG4 and tislelizumab. |
| | Administer corticosteroids at a dose of 0.5 to 2 |
| | mg/kg/day prednisone equivalents followed by |
| | corticosteroid taper for moderate (grade 2), severe |
| | (Grade 3) or life-threatening (Grade 4) transaminase |
| | elevations, with or without concomitant elevation in |
| | total bilirubin. |
| | Permanently discontinue treatment with both G9.2-17 |
| | IgG4 and tislelizumab for severe (Grade 3) or life- |
| | threatening (grade 4) immune-mediated hepatitis. |
| Immune-mediated | Administer hormone replacement as clinically |
| endocrinopathies: | indicated and corticosteroids. Withhold G9.2-17 IgG4 |
| Hypophysitis | and tislelizumab for moderate (Grade 2) hypophysitis. |
| | First administer oral prednisolone 0.5 to 1 mg/kg/day |
| | and add hormone replacement as clinically indicated |
| | until resolved/improved to ≤Grade 2. Taper |
| | corticosteroids over at least 1 month. Resume G9.2-17 |
| | IgG4 at a 50% dose reduction when resuming. |
| | Discontinuation is usually not necessary for |
| | asymptomatic and manageable patients who are treated |
| | by an endocrinologist. |
| | If ≥Grade 2 toxicity recurs, permanently discontinue |
| | treatment with both G9.2-17 IgG4 and tislelizumab. |
| | Permanently discontinue treatment G9.2-17 IgG4 and |
| | tislelizumab for severe (grade 3) and life-threatening |
| | (Grade 4) hypophysitis. |
| Adrenal insufficiency | Monitor patients for signs and symptoms of adrenal |
| | insufficiency. |
| | Withhold G9.2-17 IgG4 and tislelizumab for moderate |
| | (Grade 2) adrenal insufficiency. First administer oral |
| | prednisolone 0.5 to 1 mg/kg/day as clinically indicated |
| | until resolved/improved to ≤Grade 2. Taper |
| | corticosteroids over at least 1 month. Resume G9.2-17 |
| | IgG4 at a 50% dose reduction when resuming. |
| | Discontinuation is usually not necessary for |
| | asymptomatic and manageable patients who are treated |
| | by an endocrinologist. |
| | If ≥Grade 2 toxicity recurs, permanently |
| | discontinue treatment with both G9.2-17 IgG4 and |
| | tislelizumab. |
| | Permanently discontinue treatment G9.2-17 IgG4 |
| | and tislelizumab for severe (grade 3) and life- |
| | threatening (Grade 4) adrenal insufficiency. |
| Hypothyroidism and | Monitor thyroid function prior to and periodically |
| Hyperthyroidism | during tislelizumab treatment. |
| | Administer hormone-replacement therapy for |
| | hypothyroidism. |
| | Initiate medical management for control of |
| | hyperthyroidism. Administer oral prednisolone 0.5 |
| | mg/kg/day in patients with thyroid pain. |
| | Thyrotoxic patients should be treated with a beta |
| | blocker and may require additional treatment with a |
| | carbimazole. |
| | Hold tislelizumab for thyroiditis Grade 3 or 4 until this |
| | condition resolves to a Grade 0-1, then consider |
| | resuming treatment. |
| | Discontinue G9.2-17 IgG4 and tislelizumab for ≥Grade |
| | 3 and 4 hypothyroidism or hyperthyroidism |
| Type 1 Diabetes Mellitus | Monitor patients for hyperglycemia or other signs and |
| | symptoms of diabetes. |
| | Withhold G9.2-17 IgG4 and tislelizumab in cases of |
| | severe (Grade 3) hyperglycemia until metabolic control |
| | is achieved, until blood glucose has been stabilized to |
| | baseline or Grade 0 to 1 and the patient is |
| | hyperglycemia symptom-free. Administer insulin as |
| | clinically indicated for type 1 diabetes. |
| | Follow G9.2-17 IgG4 IMAR guidance for Type 1 |
| | Diabetes Mellitus when resolved to ≤Grade 2. |
| | Permanently discontinue treatment with both study |
| | medications for life-threatening (Grade 4) |
| | hyperglycemia. |
| Immune-mediated nephritis and | Monitor patients for renal function prior to and |
| renal dysfunction | periodically during treatment. In the event of immune- |
| (Defined as renal dysfunction | mediated nephritis, the frequency of renal function tests |
| or ≥Grade 2 increased creatinine, | should be increased. |
| requirement for corticosteroids, | Withhold G9.2-17 IgG4 and tislelizumab for moderate |
| and no clear alternate etiology) | (Grade 2) or severe (Grade 3) increased nephritis. |
| | When resolved to <Grade 2, resume G9.2-17 IgG4 at a |
| | 50% dose reduction. If ≥Grade 2 toxicity recurs, |
| | permanently discontinue treatment with both G9.2-17 |
| | IgG4 and tislelizumab. |
| | Ensure hydration and administer corticosteroids at a |
| | dose of 0.5 to 1 mg/kg/day prednisone equivalents for |
| | moderate (Grade 2) or 1 to 2 mg/kg/day prednisone |
| | equivalents for severe (Grade 3) nephritis. |
| | Permanently discontinue treatment with both G9.2-17 |
| | IgG4 and tislelizumab for life-threatening (Grade 4) |
| | nephritis and recurrent Grade 3 nephritis as well. |
| Immune-mediated skin adverse | For ≤Grade 2 rashes, treatment that can be controlled |
| reactions | or resolved with moderate strength topical or oral |
| (Tislelizumab can cause | steroids while continuing tislelizumab and G9.2-17 |
| immune-mediated rash, | IgG4. |
| including Stevens-Johnson | Monitor patients for suspected severe skin reactions |
| syndrome (SJS) and toxic | and exclude other causes. |
| epidermal necrolysis (TEN), | Withhold both medications for severe (Grade 3) until |
| some cases with fatal outcome.) | signs and symptoms become mild, and permanently |
| | discontinued for moderate (Grade 2) to severe (Grade |
| | 3) rashes that do not improve or worsen while treated |
| | with oral or IV prednisolone, and permanently |
| | discontinue for life-threatening (Grade 4) rash. |
| | For symptoms or signs of SJS or TEN, permanently |
| | discontinue tislelizumab and G9.2-17 IgG4 and refer |
| | the patient for specialized care for assessment and |
| | treatment. |
| | The recommended dose of oral prednisolone is 0.5 to 1 |
| | mg/kg/day for 3 days with tapering over 2 to 4 weeks |
| | (moderate rashes) and using methyl prednisolone 0.5 to |
| | 1 mg/kg/day with converting to oral prednisolone and |
| | tapering over at least 4 weeks (severe rashes). |
| | When resolved to <Grade 2, resume G9.2-17 IgG4 at a |
| | 50% dose reduction. |
| Immune-mediated encephalitis | Monitor for changes in neurologic function. |
| | Evaluation of patients with neurologic symptoms may |
| | include, but not be limited to, consultation with a |
| | neurologist, brain MRI, and lumbar puncture. |
| | Withhold G9.2-17 IgG4 and tislelizumab in patients |
| | with new-onset moderate to severe neurologic signs or |
| | symptoms and evaluate to rule out infectious or other |
| | causes of moderate to severe neurologic deterioration. |
| | If other etiologies are ruled out, administer |
| | corticosteroids at a dose of 1 to 2 mg/kg/day prednisone |
| | equivalents for patients with immune-mediated |
| | encephalitis, followed by corticosteroid taper. |
| | Permanently discontinue treatment with G9.2-17 IgG4 |
| | and tislelizumab for immune-mediated encephalitis. |
| Other Immune-Mediated | For any suspected immune-mediated adverse reactions, |
| Adverse Reactions | exclude other causes. |
| The following immune-mediated | Based on the severity of the adverse reaction, |
| adverse reactions occurred in | permanently discontinue or withhold G9.2-17 IgG4 and |
| less than 1% of patients treated | tislelizumab, administer high-dose corticosteroids, and |
| with tislelizumab: arthritis, | if appropriate, initiate hormone-replacement therapy. |
| encephalitis, rhabdomyolysis, | Upon improvement to Grade 1 or less, initiate |
| myositis, myocarditis, | corticosteroid taper and continue to taper over at least 1 |
| pancreatitis, and uveitis. | month. |
| | Consider restarting G9.2-17 IgG4 and tislelizumab after |
| | completion of corticosteroid taper based on the severity |
| | of the event. Resume G9.2-17 IgG4 at a 50% dose |
| | reduction. Immune-mediated adverse reactions may |
| | occur after discontinuation of tislelizumab therapy. |
| Infusion reactions | The symptoms of infusion-related reactions that may be |
| (Reported in less than 1.0% of | observed with tislelizumab include fever, chills/rigor, |
| patients in clinical trials.) | nausea, pruritus, angioedema, hypotension, headache, |
| | bronchospasm, urticaria, rash, vomiting, myalgia, |
| | dizziness, or hypertension. |
| | Patients should be closely monitored for these signs |
| | and symptoms during the infusion. |
| | For Grade 1 reactions, decrease infusion rate by 50%; |
| | for Grade 2 reactions, stop infusion and only resume |
| | with caution at 50% of previous rate if infusion-related |
| | reaction has resolved or decreased to Grade 1 in |
| | severity. For Grade 3 or Grade 4 infusion-related |
| | reactions, stop infusion and permanently discontinue |
| | tislelizumab. Subsequent infusions should be given |
| | after premedication and at the reduced infusion rate. |
| Complications of Allogeneic | Transplant-related complications include hyperacute |
| Hematopoietic Stem Cell | graft-versus-host-disease (GVHD), acute GVHD, |
| Transplantation (HSCT) | chronic GVHD, hepatic veno-occlusive disease (VOD) |
| (Fatal and other serious | after reduced intensity conditioning, and steroid- |
| complications can occur in | requiring febrile syndrome (without an identified |
| patients who receive allogeneic | infectious cause). |
| HSCT before or after being | These complications may occur despite intervening |
| treated with a PD-1 receptor | therapy between PD-1 blockade and allogeneic HSCT. |
| blocking antibody.) | Follow patients closely for evidence of transplant- |
| | related complications and intervene promptly. |
| | Consider the benefit versus risks of treatment with a |
| | PD-1 receptor blocking antibody prior to or after an |
| | allogeneic HSCT. |
| Embryo-Fetal Toxicity | Advise pregnant women of the potential risk to a fetus. |
| (Based on its mechanism of | Advise females of reproductive potential to use |
| action and data from animal | effective contraception during treatment with a |
| studies, tislelizumab can cause | tislelizumab-containing regimen and for at least 5 |
| fetal harm when administered to | months after the last dose of tislelizumab. |
| a pregnant woman.) | |

For non-IMAR, hematological and non-hematological AEs that occur in combination arms, upon assessment of causality:
If G9.2-17 IgG4 related, follow AE management instructions for G9.2-17 IgG4
If combination agent related (tislelizumab), follow management instructions in Table 7. for tislelizumab.

### Dose Modification

The decision to proceed to the next dose level of G9.2-17 IgG4 in Part 1 may be made based on safety, tolerability, and preliminary PK data obtained in at least 2 patients at the prior dose level.

The dosing schedule may also be adjusted based on PK data obtained. Detailed dose modification instructions are available as described in Tables 7-9:

TABLE 7
Recommended Dose Modifications for Tislelizumab
for AEs (other than IMARs)

| Adverse | | Dose |
|---|---|---|
| Reaction | Severity* | Modification |
| AEs | Probably related Grade 3 adverse events | Permanently |
| | Life-threatening or Grade 4 adverse | discontinue |
| | reaction | |

*Toxicity was graded per NCI CTCAE V5.

TABLE 8
Management of Immune-Mediated Adverse Reactions (IMARs) Caused by G9.2-17 IgG4

| IMAR | Guidance for Management |
|---|---|
| Immune-mediated pneumonitis | Monitor patients for signs with radiographic imaging and for |
| | clinical symptoms of pneumonitis. |
| | Withhold G9.2-17 IGG4 for moderate and permanently |
| | discontinue for severe or life-threatening pneumonitis. |
| | Administer corticosteroids at a dose of 1 to 2 mg/kg/day |
| | prednisone equivalents for moderate (Grade 2) or more |
| | severe (Grade 3-4) pneumonitis, followed by corticosteroid |
| | taper. |
| | Withhold G9.2-17 IGG4 until resolution to <Grade 2. For |
| | moderate (Grade 2) pneumonitis. Resume treatment at a |
| | 50% dose reduction. |
| | Upon recurrence of Grade 2 pneumonitis at the reduced dose |
| | of G9.2-17 IGG4, permanently discontinue G9.2-17 IGG4. |
| Immune-mediated colitis | Monitor patients for signs and symptoms of colitis. |
| | Administer corticosteroids at a dose of 0.5 to 1 mg/kg/day |
| | prednisone equivalents followed by corticosteroid taper for |
| | moderate (Grade 2) colitis of more than 5 days duration. |
| | If worsening or no improvement occurs despite initiation of |
| | corticosteroids, increase dose to 1 to 2 mg/kg/day |
| | prednisone equivalents. |
| | When initial Grade 2 colitis resolves to <Grade 2, resume |
| | G9.2-17 IGG4 at a 50% dose reduction. |
| | If Grade ≥2 colitis recurs, withhold G9.2-17 IGG4 until |
| | colitis resolves to <Grade 2. Resume G9.2-17 IGG4 at an |
| | additional 50% dose reduction. No further dose reductions |
| | for G9.2-17 IGG4 are allowed. |
| | Administer corticosteroids at a dose of 1 to 2 mg/kg/day |
| | prednisone equivalents followed by corticosteroid taper for |
| | severe (Grade 3) or life threatening (Grade 4) colitis. |
| | When Grade 3 colitis resolves to ≤Grade 2, resume G9.2-17 |
| | IGG4 at a 50% dose reduction. |
| | Permanently discontinue G9.2-17 IGG4 for life-threatening |
| | (Grade 4) or for recurrent colitis of ≥Grade 2 upon re- |
| | initiation of G9.2-17 IGG4. |
| Immune-mediated hepatitis | Monitor for changes in liver function. |
| | Administer corticosteroids at a dose of 0.5 to 1 mg/kg/day |
| | prednisone equivalents for moderate (Grade 2) transaminase |
| | elevations. |
| | Withhold G9.2-17 IGG4 for moderate (Grade 2) immune- |
| | mediated hepatitis. When resolved to <Grade 2 resume |
| | G9.2-17 IGG4 at a 50% dose reduction. |
| | If ≥Grade 2 hepatitis recurs, permanently discontinue G9.2- |
| | 17 IGG4. |
| | Administer corticosteroids at a dose of 1 to 2 mg/kg/day |
| | prednisone equivalents followed by corticosteroid taper for |
| | severe (Grade 3) or life-threatening (Grade 4) transaminase |
| | elevations, with or without concomitant elevation in total |
| | bilirubin. |
| | Permanently discontinue G9.2-17 IGG4 for severe (Grade 3) |
| | or life-threatening (Grade 4) immune-mediated hepatitis. |
| Immune-mediated | Administer hormone replacement as clinically indicated and |
| endocrinopathies: | corticosteroids at a dose of 1 mg/kg/day prednisone |
| Hypophysitis | equivalents followed by corticosteroid taper for moderate |
| | (Grade 2) or greater hypophysitis. |
| | Withhold G9.2-17 IG4 for moderate (Grade 2) or severe |
| | (Grade 3) and when resolved to <Grade 2, resume G9.2-17 |
| | IGG4 at a 50% dose reduction. |
| | If Grade ≥2 toxicity recurs, permanently discontinue G9.2- |
| | 17 IGG4. |
| | Permanently discontinue G9.2-17 IGG4 for life-threatening |
| | (Grade 4) hypophysitis. |
| Adrenal insufficiency | Monitor patients for signs and symptoms of adrenal |
| | insufficiency. |
| | Withhold G9.2-17 IGG4 for moderate (Grade 2) and |
| | once resolved to <Grade 2, resume G9.2-17 IGG4 at a |
| | 50% dose reduction. |
| | If Grade ≥2 toxicity recurs, permanently discontinue |
| | G9.2-17 IGG4. |
| | Administer corticosteroids at a dose of 1 to 2 mg/kg/day |
| | prednisone equivalents followed by a corticosteroid |
| | taper for severe (Grade 3) or life-threatening (Grade 4) |
| | adrenal insufficiency. |
| | Permanently discontinue G9.2-17 IGG4 for severe |
| | (Grade 3) or life-threatening (Grade 4) adrenal |
| | insufficiency, or for recurrent colitis of ≥Grade 2 upon |
| | re-initiation of G9.2-17 IGG4. |
| Hypothyroidism and | Monitor thyroid function prior to and periodically during |
| Hyperthyroidism | nivolumab treatment. |
| | Administer hormone-replacement therapy for |
| | hypothyroidism. |
| | Initiate medical management for control of hyperthyroidism. |
| | There are no recommended dose adjustments of G9.2-17 |
| | IGG4 for <Grade 3 hypothyroidism or hyperthyroidism |
| | Discontinue G9.2-17 IGG4 for ≥Grade 3 hypothyroidism or |
| | hyperthyroidism |
| Type 1 Diabetes Mellitus | Withhold G9.2-17 IGG4 in cases of severe (Grade 3) |
| | hyperglycemia until metabolic control is achieved. |
| | Upon resolution to ≤Grade 2, resume G9.2-17 IGG4 at a |
| | 50% dose reduction. |
| | If Grade ≥2 toxicity recurs, permanently discontinue G9.2- |
| | 17 IGG4. |
| | Permanently discontinue G9.2-17 IGG4 for life-threatening |
| | (Grade 4) hyperglycemia. |
| Immune-mediated nephritis and | Monitor patients for elevated serum creatinine prior to and |
| renal dysfunction | periodically during treatment. |
| (Defined as renal dysfunction | Administer corticosteroids at a dose of 0.5 to 1 mg/kg/day |
| or ≥Grade 2 increased creatinine, | prednisone equivalents for moderate (Grade 2) or severe |
| requirement for corticosteroids, and | (Grade 3) increased serum creatinine, if worsening or no |
| no clear alternate etiology) | improvement occurs, increase dose of corticosteroids to 1 to |
| | 2 mg/kg/day prednisone equivalents. |
| | Withhold G9.2-17 IGG4 for moderate (Grade 2) or severe |
| | (Grade 3) increased serum creatinine. When resolved |
| | to <Grade 2, resume G9.2-17 IGG4 at a 50% dose reduction. |
| | If ≥Grade 2 toxicity recurs, permanently discontinue G9.2- |
| | 17 IGG4. |
| | Administer corticosteroids at a dose of 1 to 2 mg/kg/day |
| | prednisone equivalents followed by corticosteroid taper for |
| | life-threatening (Grade 4) increased serum creatinine. |
| | Permanently discontinue G9.2-17 IGG4 for life-threatening |
| | (Grade 4) increased serum creatinine. |
| Immune-mediated skin adverse | Withhold for severe and permanently discontinue for life- |
| reactions | threatening rash. |
| (Immune-mediated rash, including | For symptoms or signs of SJS or TEN, withhold G9.2-17 |
| Stevens-Johnson syndrome (SJS) | IGG4 and refer the patient for specialized care for |
| and toxic epidermal necrolysis | assessment and treatment. |
| (TEN)) | If SJS or TEN is confirmed, permanently discontinue G9.2- |
| | 17 IGG4. |
| | For immune-mediated rash ≥Grade 2, administer |
| | corticosteroids at a dose of 1 to 2 mg/kg/day prednisone |
| | equivalents followed by a corticosteroid taper for severe |
| | (Grade 3) or life-threatening (Grade 4) rash. |
| | Withhold G9.2-17 IGG4 for moderate (Grade 2) and severe |
| | (Grade 3) rash and when resolved to <Grade 2, resume |
| | G9.2-17 IGG4 at a 50% dose reduction. |
| | If ≥Grade 2 rash recurs, withhold G9.2-17 IGG4 until |
| | resolved to <Grade 2 and resume G9.2-17 IGG4 at an |
| | additional 50% dose reduction. No further dose reductions |
| | are allowed. |
| | Permanently discontinue G9.2-17 IGG4 for life threatening |
| | (Grade 4) rash. |
| Immune-mediated encephalitis | Monitor for changes in neurologic function. |
| | Evaluation of patients with neurologic symptoms may |
| | include, but not be limited to, consultation with a |
| | neurologist, brain MRI, and lumbar puncture. |
| | Withhold G9.2-17 IGG4 in patients with new-onset |
| | moderate to severe neurologic signs or symptoms and |
| | evaluate to rule out infectious or other causes of moderate to |
| | severe neurologic deterioration. |
| | If other etiologies are ruled out, administer corticosteroids at |
| | a dose of 1 to 2 mg/kg/day prednisone equivalents for |
| | patients with immune-mediated encephalitis, followed by |
| | corticosteroid taper. |
| | Permanently discontinue G9.2-17 IGG4 for immune- |
| | mediated encephalitis. |
| Other Immune-Mediated Adverse | If uveitis occurs in combination with other immune- |
| Reactions | mediated adverse reactions, consider a Vogt-Koyanagi- |
| (May include: myocarditis, | Harada-like syndrome, and may require treatment with |
| rhabdomyolysis, myositis, uveitis, | systemic steroids to reduce the risk of permanent vision loss. |
| iritis, pancreatitis, facial and | For any suspected immune-mediated adverse reactions, |
| abducens nerve paresis, | exclude other causes. |
| demyelination, polymyalgia | Based on the severity (Grade) of the adverse reaction, |
| rheumatica, autoimmune | permanently discontinue or withhold G9.2-17 IGG4, |
| neuropathy, Guillain-Barré | administer high-dose corticosteroids, and if appropriate, |
| syndrome, hypopituitarism, | initiate hormone-replacement therapy. |
| systemic inflammatory response | Upon improvement to Grade 1 or less, initiate corticosteroid |
| syndrome, gastritis, duodenitis, | taper and continue to taper over at least 1 month. |
| sarcoidosis, histiocytic necrotizing | Consider restarting G9.2-17 IGG4 after completion of |
| lymphadenitis (Kikuchi | corticosteroid taper based on the severity of the event, |
| lymphadenitis), motor dysfunction, | resuming at a 50% dose reduction. |
| vasculitis, aplastic anemia, | |
| pericarditis, and myasthenic | |
| syndrome.) | |

TABLE 9
Recommended Dose Modifications for G9.2-17 IgG4
(AEs outside the DLT window and other than IMARs)

| Adverse Reaction | Severity | G9.2-17 IgG4 Dose Modification |
|---|---|---|
| Non-hematologic | ≥Grade 4 | Permanently discontinue treatment |
| and hematologic | | with G9.2-17 IgG4. |
| toxicities | | |
| Hematologic | Grade 3 | Withhold treatment until toxicity |
| toxicities | | resolves to ≤Grade 2. |
| | | Resume G9.2-17 IgG4 at 50% |
| | | reduction from previous dose. |
| | | If recurs again at ≥Grade 3 severity, |
| | | hold until resolved to ≤Grade 2 and |
| | | implement a further 50% dose |
| | | reduction and medical monitor. |
| | | No further dose reductions are |
| | | allowed. |
| Non-hematologic | Grade 3 | Withhold treatment until toxicity |
| toxicities | | resolves to ≤Grade 2. |
| (except those listed | | Resume G9.2-17 IgG4 at 50% |
| in Table 8, | | reduction from previous dose. |
| Management of | | If recurs again at ≥Grade 3 severity, |
| Immune-Mediated | | hold until resolved to ≤Grade 2 and |
| Adverse Reactions | | implement a further 50% dose |
| (IMARs) Caused | | reduction and medical monitor. |
| by G9.2-17 IgG4) | | No further dose reductions are |
| | | allowed. |
| Gastrointestinal | Grade 3 | Withhold G9.2-17 IgG4 until toxicity |
| (Nausea, Vomiting) | Not requiring | reverses to ≤Grade 2 |
| | hospitalization or parenteral | Resume G9.2-17 IgG4 with no dose reduction |
| | nutrition support; managed | If recurrent, withhold G9.2-17 IgG4 |
| | by supportive care | until toxicity reverses to ≤Grade 2 |
| Electrolyte | Grade 3 corrected | Then resume G9.2-17 IgG4 at a 50% |
| Abnormalities | to ≤Grade 2 within 24 hours | reduction from the previous dose |
| Electrolyte | ≥Grade 3 lasting <24-72 hours | If recurrent, withhold G9.2-17 IgG4 |
| Abnormalities | Not clinically complicated; | until toxicity reverses to ≤Grade 2 |
| | resolves spontaneously or | Resume G9.2-17 IgG4 at an |
| | responds to conventional | additional 50% dose reduction |
| | medical interventions | No further dose reductions are |
| Amylase or Lipase | ≥Grade 3 | allowed |
| Elevation | Not associated with | |
| | symptoms or clinical | |
| | manifestations of | |
| | pancreatitis | |

### Dose Administration and Dose Delay

If an infusion-related reaction is encountered, interrupt the infusion and if clinically indicated, administer relevant medication(s) (eg, anti-histamine, anti-emetic, steroids, anti-pyretics, beta-blocker(s) etc.). If it is deemed appropriate to resume the infusion, resume at a slower infusion rate.

For subsequent cycles for the same patient, apply the appropriate pre-medications (anti-histamine, anti-emetic, steroids, anti-pyretics, beta-blocker(s) etc., as clinically indicated needed) and consider utilizing a slower infusion rate.

If any clinically meaningful AE≥Grade 3 possibly related or related to one or more study drugs occurs, it will be discussed with the Medical Monitor before continuing with dosing. A a dose delay may be necessary for ≥Grade 3 AE.

### Dose Reduction

No dose reductions may be allowed for any patient that is being evaluated for DLTs (within the 28 day DLT window). In case a dose reduction is necessary, the study intervention will be administered as follows:

For Parts 1 and 2 G9.2-17 IgG4 alone patients: Dose reduction may be allowed when it is assessed that clinical benefit is being derived and may continue to be derived under dose reduced conditions, see Table 8 (for IMARs) or Table 9 (for other AEs).

For Part 2 combination treatment cohorts of G9.2-17 IgG4 or G9.2-17 IgG4 tislelizumab, experience derived from clinical trials of the approved drug, as summarized in the approved product labeling, will inform the adverse event management including guidance on delaying, dose-reducing and/or withholding entirely tislelizumab, see Table 6-7 and 9.

### Dose Modifications for Specific AEs Related to Administration of Tislelizumab

Recommendations for tislelizumab modifications based on specific AEs are provided in Table 6 (for IMARs) and Table 7 (for other AEs).

### Dose Modification for IMARs

If an IMAR occurs, refer to Table 8 and Table 6 for guidance on dose management of G9.2-17 IgG4 and/or tislelizumab. All relevant medical exam(s)/test(s) in order to confirm that the adverse event is an IMAR.

### Discontinuation of Study Intervention

In rare instances, it may be necessary for a patient to permanently discontinue study intervention. If study intervention is permanently discontinued due to reasons other than disease progression, and the patient is not being treated with other anti-cancer therapy(ies), the patient will continue to be evaluated for disease progression for up to 2 years. See the Schedule of Assessment table for data to be collected at the time of discontinuation of study intervention and follow-up and for any further evaluations that need to be completed.

Every effort must be made by study personnel to keep patients on study treatment until one of the reasons for study treatment termination are met (disease progression, toxicity related to the study drug, withdrawal of consent). If the patient has radiographic progression but no unequivocal clinical progression and alternate treatment is not initiated, the patient may continue on study treatment. However, if patients have unequivocal clinical progression without radiographic progression, study treatment should be stopped and patients advised regarding available treatment options.

A patient may be discontinued prior to disease progression for any of the following reasons:
A DLT per definition provided herein
An AE occurs/recurs outside of the DLT window that requires discontinuation of study treatment(s)
An IMAR occurs/recurs that requires discontinuation of study treatment(s)
Intercurrent illness or medical condition that prevents further administration of treatment or may jeopardize the patient's safety if they continue on study treatment
Pregnancy
Use of a non-protocol anti-cancer therapy

Patients may also be discontinued prior to disease progression for any of the following reasons:
Significant deviation from protocol on the part of the patient (includes lack of compliance)

### Concomitant Therapy

Any medication or vaccine (including over-the-counter or prescription medicines, recreational drugs, vitamins, and/or herbal supplements) that the participant is receiving at the time of enrollment or receives during the study must be recorded along with:
Reason for use
Dates of administration including start and end dates
Dosage information including dose and frequency

### Permitted Medications

The following concomitant medications are allowed:
Any standard of care pre-medication for patients on a combination treatment regimen.
Continuation of bisphosphonate treatment (eg, zoledronic acid) or denosumab for bone metastases, which have been stable for at least 6 months before treatment (C1D1),
The use of inhaled corticosteroids and mineralocorticoids (eg, fludrocortisone), topical steroids, intranasal steroids, intra-articular, and ophthalmic steroids
Prophylactic or therapeutic use of anticoagulants
Vaccination for COVID-19, common flu and/or other common clinically required indications (eg tetanus, pneumococcus, HBV, etc.) is allowed before or during the study period. The timing and type of vaccine must be recorded.

### Prohibited Medications

Following medications are not allowed while on this study:
Concomitant administration of other investigational agents, other than G9.2-17 IGG4, for any indication
Systemic immunosuppressive treatment, including, but not limited to cyclophosphamide, azathioprine, methotrexate, thalidomide, and anti-TNF agents. However, patients are allowed to take acute, low dose systemic immunosuppressant medications (eg, ≤10 mg/day of prednisone or equivalent).
Replacement therapy (eg, thyroxine, insulin, physiologic corticosteroid replacement therapy [eg, ≤10 mg/day of prednisone equivalent] for adrenal or pituitary insufficiency) is not considered a form of systemic treatment.

### Efficacy Assessments

Planned time points for all efficacy assessments are provided in the table of Schedule of Assessment.

### RECIST v1. Criteria for Tumor Assessment

At the screening tumor assessment, tumor lesions/lymph nodes will be categorized as measurable or non-measurable with measurable tumor lesions recorded according to the longest diameter in the plane of measurement (except for pathological lymph nodes, which are measured in the shortest axis). When more than one measurable lesion is present at screening all lesions up to a maximum of five lesions total (and a maximum of two lesions per organ) representative of all involved organs should be identified as target lesions. Target lesions should be selected on the basis of their size (lesions with the longest diameter). A sum of the diameters for all target lesions may be calculated and reported as the baseline sum diameters.

All other lesions (or sites of disease) including pathological lymph nodes should be identified as non-target lesions and should also be recorded at screening. Measurements are not required, and these lesions should be followed as ‘present’, ‘absent’, or ‘unequivocal progression’. Tumor target lesions will be assessed according to the RECIST v1.1 Guidelines (Eisenhauer et al., 2009) using the following disease response measures.
Complete response (CR): Disappearance of all target lesions. Any pathological lymph nodes (whether target or non-target) must have reduction in short axis to <10 mm.
Partial response (PR): At least a 30% decrease in the sum of diameters of target lesions, taking as reference the baseline sum diameters.
Stable disease (SD): Neither sufficient shrinkage to qualify for PR nor sufficient increase to qualify for PD, taking as reference the smallest sum diameters while on study.
Progressive disease: At least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions is also considered progression).

The following guidelines can be used for evaluating non-target lesions. See also Table 10 below.
Complete Response (CR): Disappearance of all non-target lesions and normalization of tumor marker level. All lymph nodes must be non-pathological in size (≤10 mm short axis).
Non-CR/Non-PD: Persistence of one or more non-target lesion(s) and/or maintenance of tumor marker level above the normal limits.
Progressive Disease (PD): Unequivocal progression of existing non-target lesions. (Note: the appearance of one or more new lesions is also considered progression).

The disease response measures at different timepoints will allow for the calculation of the following:
Disease control rate (DCR), defined as percentage of patients who have achieved CR, PR and SD.
Objective response rate (ORR), defined as the proportion of patients with tumor size reduction of a predefined amount (tumor shrinkage of ≥30%).
Progression-free survival (PFS), defined as the time from study drug treatment initiation to disease progression (tumor growth by ≥30%).
Duration of response (DoR), defined as the length of time that a tumor continues to respond to treatment without the cancer growing or spreading.
Overall survival (OS) defined as defined as the time from study drug treatment initiation to death from any cause.

TABLE 10
Evaluation of Overall Timepoint Response for
Patients with Measurable Disease at Baseline

| Target | Non-target | New | Overall |
|---|---|---|---|
| Lesions | Lesions | Lesions | Response |
| CR | CR | No | CR |
| CR | Non-CR/Non-PD | No | PR |
| CR | NE | No | PR |
| PR | Non-PD or NE | No | PR |
| SD | Non-PD or NE | No | SD |
| Not all evaluated | Non-PD | No | NE |
| Progressive | Any | Yes or No | Progressive |
| disease | | | disease |
| Any | Progressive | Yes or No | Progressive |
| | disease | | disease |
| Any | Any | Yes | Progressive |
| | | | disease |

CR: Complete Response,
Non-PD: Non-progressive Disease,
PR: Partial Response,
SD: Stable Disease,
NE: Non-evaluable
*When target lesions show SD/PR and some subset of non-target lesions is non-evaluable, a careful decision must be made whether to call the overall response at this timepoint SD/PR or NE. This is based on whether the non- evaluable lesions, if they showed growth, could cause an overall response of progressive disease in the context of the other lesion responses seen. If the non-evaluable non-target lesions comprise a significant proportion of the overall disease burden, the appropriate timepoint response is NE.

### Adverse Event Management

AEs may not be recorded prior to the administration of the first dose of study medication. AEs that start, or symptoms related to medical history that worsen after study drug administration will be recorded. AEs should be followed until they are either resolved, have returned to baseline, or are determined to be a stable or chronic condition. All SAEs are to be collected until 30 days after the last dose of study medication

### Immune-Mediated Adverse Reactions

Immune-mediated adverse reactions (IMARs) are identified for tislelizumab.

The specific IMARs noted are:
Immune-Mediated Hepatitis
Immune-Mediated Nephritis
Immune-Mediated Pneumonitis
Immune-Mediated Pneumonitis
Immune-Mediated Colitis and Diarrhea Immune-Mediated Endocrinopathies
Immune-Mediated Skin Reactions
Other Immune-Mediated Adverse Reactions: arthritis, encephalitis, rhabdomyolysis, myositis, myocarditis, pancreatitis, and uveitis.

The monitoring plan is intended to limit the severity and duration of IMARs that occur during combination drug development, and encompass: scheduled visits for a physical exam, vital signs, safety laboratory assessments including blood hematology, biochemistry, assessing endocrine functions each Day 1 of a new dosing cycle (pre-dose), assessing coagulation status and urine analyses. The Schedule of Assessments (see Example 1) also encompasses assessing the ejection fraction once every three months and conducting regular ECGs.

All relevant medical exam(s)/test(s) will be carried out in order to confirm that the adverse event is an IMAR.

Instructions for the management of these IMARs are included in Table 8 for G9.2-17 IgG4 alone, and Table 6 for the G9.2-17 IgG4+tislelizumab combination treatment.

### EQUIVALENTS

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

While several inventive embodiments have been described and illustrated herein, those of ordinary skill in the art are readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art are readily appreciate that all parameters, dimensions, materials, and configurations described herein are meant to be exemplary and that the actual parameters, dimensions, materials, and/or configurations are dependent upon the specific application or applications for which the inventive teachings is/are used. Those skilled in the art are recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” are refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A method for treating a solid tumor, the method comprising administering to a subject in need thereof
(a) an effective amount of an antibody that binds human Galectin-9 (anti-Galectin-9 antibody) and
(b) an effective amount of tislelizumab,
wherein the anti-Galectin-9 antibody comprises:
(i) a light chain variable region (V_{L}) comprising a light chain complementarity determining region 1 (CDR1) set forth as SEQ ID NO: 1, a light chain complementarity determining region 2 (CDR2) set forth as SEQ ID NO: 2, and a light chain complementarity determining region 3 (CDR3) set forth as SEQ ID NO: 3, and
(ii) a heavy chain variable region comprising a heavy chain complementarity determining region 1 (CDR1) set forth as SEQ ID NO: 4, a heavy chain complementarity determining region 2 (CDR2) set forth as SEQ ID NO: 5, and a heavy chain complementarity determining region 3 (CDR3) set forth as SEQ ID NO: 6 and
wherein the anti-Galectin-9 antibody is administered to the subject at a dose of about 0.2 mg/kg to about 18 mg/kg.

2. The method of claim 1, wherein the solid tumor is head and neck cancer, urothelial cancer, gastric esophageal cancer, or non-small cell lung cancer.

3. The method of claim 1, wherein the solid tumor is a metastatic tumor.

4. The method of claim 1, wherein the anti-Galectin-9 antibody is administered to the subject at a dose of about 4 mg/kg to about 18 mg/kg.

5. The method of claim 4, wherein the anti-Galectin-9 antibody is administered to the subject at a dose of about 4 mg/kg, about 6.3 mg/kg, about 10 mg/kg, about 12 mg/kg, about 14 mg/kg, about 16 mg/kg, or about 18 mg/kg, optionally wherein the dose of the anti-Galecti-9 antibody is about 6.3 mg/kg, about 10 mg/kg, or about 16 mg/kg.

6. The method of claim 1, wherein the anti-Galectin-9 antibody is administered to the subject once a week.

7. The method of claim 6, wherein the anti-Galectin-9 antibody is administered to the subject at a dose of about 6.3 mg/kg, about 10 mg/kg once a week, or about 16 mg/kg.

8. The method of claim 1, wherein the anti-Galectin-9 antibody is administered to the subject by intravenous infusion.

9. The method of claim 1, wherein the tislelizumab is administered to the subject at a dose of about 200 mg once every 3 weeks, at a dose of about 300 mg every 4 weeks, or at a dose of about 400 mg every six weeks.

10. The method of claim 9, wherein the tislelizumab is administered to the subject at a dose of about 300 mg every 4 weeks.

11. The method of claim 9, wherein the tislelizumab is administered to the subject by intravenous infusion.

12. The method of claim 1, wherein the anti-Galectin-9 antibody is administered to the subject at about 6.3 mg/kg once a week by intravenous infusion and the tislelizumab is administered to the subject at a dose of about 300 mg every 4 weeks by intravenous infusion.

13. The method of claim 1, wherein the anti-Galectin-9 antibody is administered to the subject at about 10 mg/kg once a week by intravenous infusion and the tislelizumab is administered to the subject at a dose of about 300 mg every 4 weeks by intravenous infusion.

14. The method of claim 1, wherein the anti-Galectin-9 antibody is administered to the subject at about 16 mg/kg once a week by intravenous infusion and the tislelizumab is administered to the subject at a dose of about 300 mg every 4 weeks by intravenous infusion.

15. The method of claim 1, wherein the tislelizumab is administered to the subject on a day when the subject receives the anti-Galectin 9 antibody or wherein the administration of tislelizumab and the administration of the anti-Galectin 9 antibody are on two consecutive days.

16. The method of claim 1, wherein the administration of tislelizumab is performed prior to the administration of the anti-Galectin 9 antibody.

17. The method of claim 1, wherein the subject is a human patient having the solid tumor.

18. The method of claim 1, wherein the V_{L} of the anti-Galectin-9 antibody comprises the amino acid sequence of SEQ ID NO: 8, and wherein the V_{H} of the anti-Galectin-9 antibody comprises the amino acid sequence of SEQ ID NO: 7.

19. The method of claim 18, wherein the anti-Galectin-9 antibody is an IgG1 or IgG4 molecule.

20. The method of claim 19, wherein the anti-Galectin-9 antibody is an IgG4 molecule having a modified Fc region of human IgG4.

21. The method of claim 20, wherein the modified Fc region of human IgG4 comprises the amino acid sequence of SEQ ID NO: 14.

22. The method of claim 1, the anti-Galectin-9 antibody comprises a heavy chain comprising the amino acid sequence of SEQ ID NO: 19 and a light chain comprising the amino acid sequence of SEQ ID NO: 15.

23. The method of claim 1, wherein the subject has undergone one or more prior anti-cancer therapies.

24. The method of claim 23, wherein the one or more prior anti-cancer therapies comprise chemotherapy, immunotherapy, radiation therapy, a therapy involving a biologic agent, or a combination thereof.

25. The method of claim 23, wherein the subject has progressed disease through the one or more prior anti-cancer therapies, or is resistant to the one or more prior therapies.

26. The method of claim 1, wherein the subject is a human patient having an elevated level of Galectin-9 relative to a control value.

27. The method of claim 26, wherein the human patient has an elevated serum or plasma level of Galectin-9 relative to the control value.

28. The method of claim 1, wherein the human patient has cancer cells expressing Galectin-9, immune cells expressing Galectin-9, or both.

29. The method of claim 1, further comprising monitoring occurrence of adverse effects in the subject.

30. The method of claim 29, further comprising reducing the dose of the anti-Galectin-9 antibody, the dose of tislelizumab, or both, when an adverse effect occurs.


SEQUENCE LISTING (23 sequences)

SEQ ID NO: 1 - protein, 11 aa, synthetic construct
RASQSVSSAVA

SEQ ID NO: 2 - protein, 7 aa, synthetic construct
SASSLYS

SEQ ID NO: 3 - protein, 9 aa, synthetic construct
QQSSTDPIT

SEQ ID NO: 4 - protein, 9 aa, synthetic construct
FTVSSSSIH

SEQ ID NO: 5 - protein, 17 aa, synthetic construct
YISSSSGYTYYADSVKG

SEQ ID NO: 6 - protein, 15 aa, synthetic construct
YWSYPSWWPYRGMDY

SEQ ID NO: 7 - protein, 124 aa, synthetic construct
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSS

SEQ ID NO: 8 - protein, 108 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSSTDPITFGQGTKVEIKR

SEQ ID NO: 9 - protein, 20 aa, synthetic construct
MYRMQLLSCIALSLALVTNS

SEQ ID NO: 10 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 106 aa, synthetic construct
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 14 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 15 - protein, 214 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSSTDPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 454 aa, synthetic construct
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 454 aa, synthetic construct
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 451 aa, synthetic construct
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 19 - protein, 451 aa, synthetic construct
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 20 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 451 aa, synthetic construct
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 451 aa, synthetic construct
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK